(19) United States
(12) Patent Application Publication
(10) Pub. No.: US 2023/0331655 A1
(43) Pub. Date: Oct. 19, 2023

(54) HYDROXYNORKETAMINE COMPOUNDS AND METHODS OF USE THEREOF

(71) Applicants: UNIVERSITY OF MARYLAND, BALTIMORE, Baltimore, MD (US); THE UNITED STATES OF AMERICA, as represented by THE SECRETARY, DEPARTMENT OF HEALTH AND HUMAN SERVIC, Bethesda, MD (US); THE UNITED STATES GOVERNMENT as represented by THE DEPARTMENT OF VETERANS AFFAIRS, Washington, DC (US)

(72) Inventors: Todd GOULD, Elkridge, MD (US); Jaclyn HIGHLAND, Karns City, PA (US); Panos ZANOS, Strovolos, Nicosia (CY); Craig THOMAS, Gaithersburg, MD (US); Patrick MORRIS, Laurel, MD (US)

(21) Appl. No.: 18/043,163

(22) PCT Filed: Aug. 27, 2021
(86) PCT No.: PCT/US2021/048095

§ 371 Date: Feb. 27, 2023

Related U.S. Application Data
(60) Provisional application No. 63/071,334, filed on Aug. 27, 2020.

(51) Int. Cl. C07C 225/20; A61P 25/24

(52) U.S. Cl. CPC C07C 225/20; A61P 25/24; C07C 2601/14

(57) ABSTRACT

Compounds and methods of using the same for treating diseases and conditions including a depressive disorder, an anxiety disorder, drug addiction, schizophrenia, Alzheimer's dementia, amyotrophic lateral sclerosis, complex regional pain syndrome (CRPS), chronic pain, neuropathic pain, anhedonia, and fatigue are provided.

### STATEMENT AS TO FEDERALLY SPONSORED RESEARCH

This invention was made with government support under Grant Number MH107615 awarded by the National Institutes of Health. The government has certain rights in the invention.

### FIELD

The disclosure relates generally to compounds and methods of using the same for treating diseases and conditions including a depressive disorder, an anxiety disorder, drug addiction, schizophrenia, Alzheimer's dementia, amyotrophic lateral sclerosis, complex regional pain syndrome (CRPS), chronic pain, neuropathic pain, anhedonia, and fatigue.

### BACKGROUND

Ketamine, a drug currently used in human anesthesia and veterinary medicine, has been shown in clinical studies to be effective in the treatment of several conditions, including treatment-resistant bipolar depression, major depressive disorder, neuropathic pain, and chronic pain, including complex regional pain syndrome (CRPS).

The need for therapeutics that exhibit the therapeutic properties of ketamine with efficacy in a higher percentage of patients, reduced anesthetic properties and reduced abuse liability exists. The present disclosure fulfills this need and provides additional advantages set forth herein.

### SUMMARY

The disclosure provides a compound of any one of formula (I), formula (II), or formula (III), or a pharmaceutically acceptable salt, solvate, hydrate, cocrystal, or prodrug thereof:

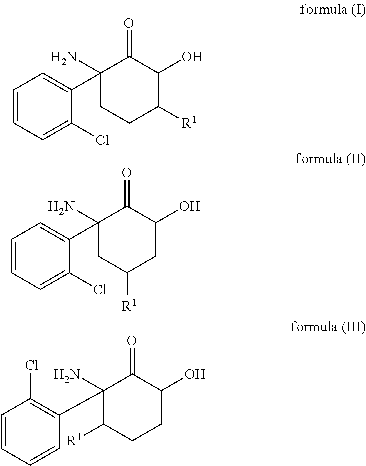

wherein in formula (I), formula (II), and formula (III):
R¹ is independently selected at each occurrence from halogen, hydroxyl, amino, cyano, C₁-C₄alkyl, C₁-C₄alkoxy, mono- and di-C₁-C₄alkylamino, C₁-C₂haloalkyl, and C₁-C₂haloalkoxy.

In some embodiments, the compound of formula (I) is a compound of any one of formula (Ia), formula (Ib), formula (Ic), or formula (Id), or a pharmaceutically acceptable salt, solvate, hydrate, cocrystal, or prodrug thereof:

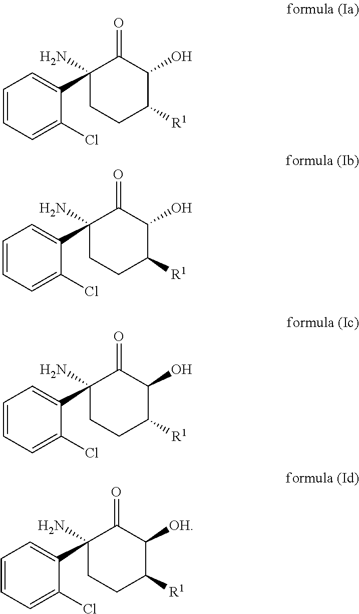

In some embodiments, the compound of formula (II) is a compound of any one of formula (IIa), formula (IIb), formula (IIc), or formula (IId), or a pharmaceutically acceptable salt, solvate, hydrate, cocrystal, or prodrug thereof:

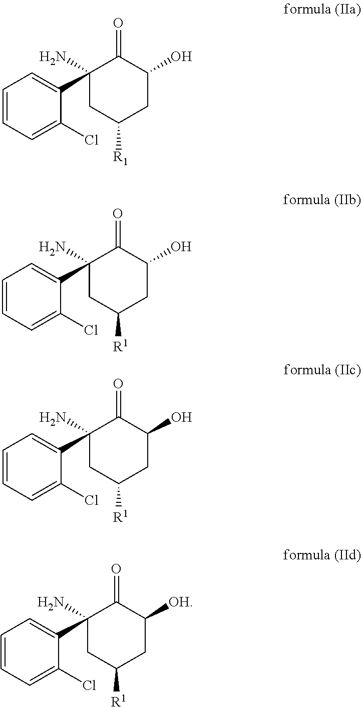

In some embodiments, the compound of formula (III) is a compound of any one of formula (IIIa), formula (IIIb), formula (IIIc), or formula (IIId), or a pharmaceutically acceptable salt, solvate, hydrate, cocrystal, or prodrug thereof:

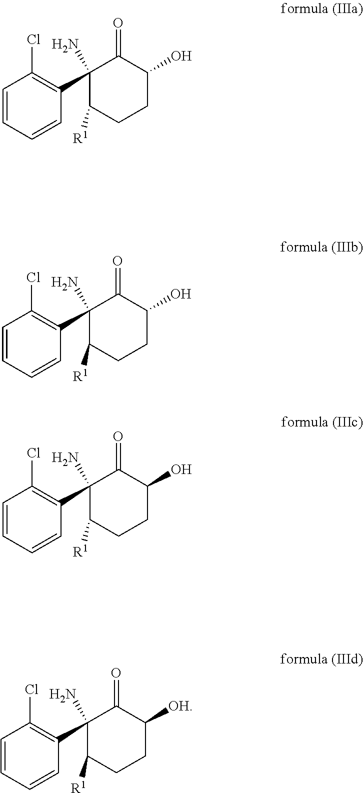

In some embodiments, R¹ is independently selected at each occurrence from C₁-C₄alkyl. In some embodiments, R¹ is methyl.

In some embodiments, the compound is of any one of formulas 1001-1084, or a pharmaceutically acceptable salt, solvate, hydrate, cocrystal, or prodrug thereof.

In some embodiments, the compound is compound 1001, or a pharmaceutically acceptable salt, solvate, hydrate, cocrystal, or prodrug thereof.

The disclosure also provides a pharmaceutical composition for treating a disease or condition selected from a depressive disorder, an anxiety disorder, drug addiction, schizophrenia, Alzheimer's dementia, amyotrophic lateral sclerosis, complex regional pain syndrome (CRPS), chronic pain, neuropathic pain, anhedonia, and fatigue. In some embodiments, the pharmaceutical composition includes a compound of any one of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1084, or formulas 2001-2004, or a pharmaceutically acceptable salt, solvate, hydrate, cocrystal, or prodrug thereof, and a pharmaceutically acceptable carrier. In some embodiments, the depressive disorder is selected from major depressive disorder, bipolar disorder, postpartum depression, drug withdrawal induced depression, treatment refractory depression, persistent depressive disorder (dysthymia), perinatal depression, seasonal affective disorder, seasonal depression, premenstrual dysphoric disorder, psychotic depression, suicidal ideation, suicidal actions, disruptive mood dysregulation disorder, premenstrual dysphoric disorder, substance/medication-induced depressive disorder, depressive disorder due to another medical condition, other specified depressive disorder, and an unspecified depressive disorder. In some embodiments, the anxiety disorder is selected from general anxiety disorder, obsessive-compulsive disorder (OCD), post-traumatic stress disorder (PTSD), separation anxiety disorder, selective mutism, specific phobia, social anxiety disorder (social phobia), panic disorder, panic attack (specifier), agoraphobia, substance/medication-induced anxiety disorder, anxiety disorder due to another medical, other specified anxiety disorder, anhedonia, and unspecified anxiety disorder. In some embodiments, the drug addiction is selected from nicotine addiction, alcohol addiction, cannabis addiction, cocaine addiction, and opioid addiction. In some embodiments, the drug addiction is opioid addiction. In some embodiments, the pharmaceutical composition is formulated for oral administration.

The disclosure also provides a method of treating or preventing a disease or condition selected from a depressive disorder, an anxiety disorder, drug addiction, schizophrenia, Alzheimer's dementia, amyotrophic lateral sclerosis, complex regional pain syndrome (CRPS), chronic pain, neuropathic pain, anhedonia, and fatigue. In some embodiments, the method comprising administering to the patient a therapeutically effective amount of a compound of any one of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1084, or formulas 2001-2004, or a pharmaceutically acceptable salt, solvate, hydrate, cocrystal, or prodrug thereof. In some embodiments, the depressive disorder is selected from major depressive disorder, bipolar disorder, postpartum depression, drug withdrawal induced depression, treatment refractory depression, persistent depressive disorder (dysthymia), perinatal depression, seasonal affective disorder, seasonal depression, premenstrual dysphoric disorder, psychotic depression, suicidal ideation, suicidal actions, disruptive mood dysregulation disorder, premenstrual dysphoric disorder, substance/medication-induced depressive disorder, depressive disorder due to another medical condition, other specified depressive disorder, and an unspecified depressive disorder. In some embodiments, the anxiety disorder is selected from general anxiety disorder, obsessive-compulsive disorder (OCD), post-traumatic stress disorder (PTSD), separation anxiety disorder, selective mutism, specific phobia, social anxiety disorder (social phobia), panic disorder, panic attack (specifier), agoraphobia, substance/medication-induced anxiety disorder, anxiety disorder due to another medical, other specified anxiety disorder, anhedonia, and unspecified anxiety disorder. In some embodiments, the drug addiction is selected from nicotine addiction, alcohol addiction, cannabis addiction, cocaine addiction, and opioid addiction. In some embodiments, the drug addiction is opioid addiction. In some embodiments, the pharmaceutical composition is formulated for oral administration. In some embodiments, the compound is a compound of formula 2001, or a pharmaceutically acceptable salt, solvate, hydrate, cocrystal, or prodrug thereof. In some embodiments, the compound is a compound of formula 2002, or a pharmaceutically acceptable salt, solvate, hydrate, cocrystal, or prodrug thereof.

### BRIEF DESCRIPTION OF THE FIGURES

FIG. 1A-FIG. 1D illustrate experimental data demonstrating that (2,6)-hydroxynorketamines reduce immobility time in the mouse forced swim test. (2R,6R)-hydroxynorketamine (FIG. 1A; HNK; administered as HCl salt), (2S,6S)-HNK (FIG. 1B; administered as HCl salt), (2R,6S)-HNK (FIG. 1C; free base dose), and (2S,6R)-HNK (FIG. 1D; free base dose) reduced immobility time in the mouse forced swim test with minimal effective doses of 10 mg/kg, 30 mg/kg, 1 mg/kg, and 3 mg/kg, respectively, 24 h after intraperitoneal injection. Graphs and error bars represent mean and SEM, respectively, of results obtained from 10-18 mice/group. *p<0.05, **p<0.01, ***p<0.001.

FIG. 2A-FIG. 2F illustrate that (5R)-methyl-(2R,6R)-hydroxynorketamine reduces forced swim test immobility time. Three-dimensional structures of (2R,6R)-hydroxynorketamine (FIG. 2A), (HNK; adapted from Morris et al., 2017), (2R,6S)-HNK (adapted from Morris et al., 2017) (FIG. 2B), and (5R)-methyl(Me)-(2R,6R)-HNK (FIG. 2C). FIG. 2D and FIG. 2E illustrate experimental data demonstrating plasma (FIG. 2D) and brain (FIG. 2E) concentrations of (5R)-Me-(2R,6R)-HNK following intraperitoneal administration (5 mg/kg HCl salt; equivalent to 4.37 mg/kg free base dose). Inset area-under-the-curve (AUC) of the plots of concentration vs. time.

FIG. 2F illustrates experimental data demonstrating that (5R)-Me-(2R,6R)-HNK (dosed as HCl salt) reduces immobility time in the mouse forced swim test 24 hours after treatment, with minimal effective dose of 1 mg/kg (i.p.). Data are the mean±SEM. n=13-15/group. *p<0.05, **p<0.01.

FIG. 3 illustrates the three dementional structure of (5R)-methyl(Me)-(2R,6R)-HNK.

FIG. 4A-FIG. 4N illustrate the plasma levels of hydroxynorketamines in mice. Plasma concentrations following intraperitoneal administration of (FIG. 4A) (2R,6R)-hydroxynorketamine (HNK; 4.3 mg/kg free base dose), (FIG. 4B) (2S,6S)-HNK (4.3 mg/kg free base dose), (FIG. 4C) (2R,6S)-HNK (5 mg/kg), (FIG. 4D) (2S,6R)-HNK (5 mg/kg), (FIG. 4E) (2R,5R)-HNK (5 mg/kg), (FIG. 4F) (2S,5S)-HNK (5 mg/kg), (FIG. 4G) (2R,5S)-HNK (5 mg/kg), (FIG. 4H) (2S,5R)-HNK (5 mg/kg), (FIG. 4I) (2R,4R)-HNK (5 mg/kg), (FIG. 4J) (2S,4S)-HNK (5 mg/kg), (FIG. 4K) (2R,4S)-HNK (5 mg/kg), and (FIG. 4L) (2S,4R)-HNK (4.3 mg/kg free base dose) to male (M; solid lines) and female (F; dashed lines) mice. Inset area-under-the-curve (AUC) of the plots of concentration vs. time. FIG. 4M is a graph of experimental data demonstrating dose normalized plasma AUC concentrations (Cₘₐₓ) for the 12 HNKs. FIG. 4N is a graph of experimental data demonstrating dose normalized peak plasma concentrations (Cₘₐₓ) for the 12 HNKs. Data points and error bars represent mean and SEM, respectively, of results obtained from 3-4 mice/group. *p<0.05, **p<0.01, ***p<0.001.

FIG. 5A-FIG. 5N illustrate brain levels of hydroxynorketamines in mice. Plasma concentrations following intraperitoneal administration of (FIG. 5A) (2R,6R)-hydroxynorketamine (HNK; 4.3 mg/kg free base dose), (FIG. 5B) (2S,6S)-HNK (4.3 mg/kg free base dose), (FIG. 5C) (2R,6S)-HNK (5 mg/kg), (FIG. 5D) (2S,6R)-HNK (5 mg/kg), (FIG. 5E) (2R,5R)-HNK (5 mg/kg), (FIG. 5F) (2S,5S)-HNK (5 mg/kg), (FIG. 5G) (2R,5S)-HNK (5 mg/kg), (FIG. 5H) (2S,5R)-HNK (5 mg/kg), (FIG. 5I) (2R,4R)-HNK (5 mg/kg), (FIG. 5J) (2S,4S)-HNK (5 mg/kg), (FIG. 5K) (2R,4S)-HNK (5 mg/kg), and (FIG. 5L) (2S,4R)-HNK (4.3 mg/kg free base dose) to male (M; solid lines) and female (F; dashed lines) mice. Inset area under the concentration vs. time curve (AUC). FIG. 5M is a graph of experimental data demonstrating dose normalized brain AUC concentrations (Cₘₐₓ) for the 12 HNKs. FIG. 5N is a graph of experimental data demonstrating dose normalized peak brain concentrations (Cₘₐₓ) for the 12 HNKs. Data points and error bars represent mean and SEM, respectively, of results obtained from 3-4 mice/group. *p<0.05, **p<0.01, ***p<0.001.

FIG. 6 provides data demonstrating that (2R,6R)-HNK prevents conditioning to low doses of morphine in stress susceptible mice.

FIG. 7 provides data showing that the administration of (2R,6R)-HNK did not prevent the development of high-dose (5 mg/kg) morphine CPP in mice.

FIGS. 8A-8C provide data demonstrating that (2R,6R)-HNK prevents conditioned-place aversion (dysphoria) induced by acute morphine abstinence. FIG. 8A shows data for mice injected with saline. FIG. 8B shows data for mice injected with morphine. FIG. 8C shows data for the change from pre-conditioning to post-conditioning. FIG. 8D shows the timeline of the study over Days 1-6.

FIGS. 9A-9H provide data demonstrating scored behaviors.

FIG. 10 provides data demonstrating the average withdrawal score calculated from the different behaviors shown in FIGS. 9A-9H. FIG. 10 shows that (2R,6R)-HNK decreases somatic symptoms following naloxone-precipitated morphine withdrawal.

FIGS. 11A-11H provide data demonstrating that (2R,6R)-HNK reverses emotional deficits induced by stress during morphine abstinence and prevents reinstatement following recovery. FIGS. 11A-11D illustrate post-subthreshold social defeat susceptibility (1-2 days post-stress) in social interaction (FIG. 11A), sucrose preference (FIG. 11B), female urine sniffing (FIG. 11C), and untorn nestlet weight (FIG. 11D). FIGS. 11E-11F illustrate recovery assessment (10 days post-stress) in sucrose preference (FIG. 11E) and untorn nestlet weight (FIG. 11F).

FIGS. 11G-11H illustrate post-stress reinstatement (1 min exposure—all mice) in sucrose preference (FIG. 11G) and untorn nestlet weight (FIG. 11H).

FIG. 12 provides data demonstrating that (2R,6R)-HNK decreases somatic symptoms following naloxone-precipitated morphine withdrawal.

FIG. 13 provides a timeline to a study demonstrating that (2R,6R)-HNK reverses emotional deficits induced by stress during morphine abstinence and prevents reinstatement following recovery.

### DETAILED DESCRIPTION

Unless defined otherwise, all technical and scientific terms used herein have the same meaning as is commonly understood by one of skill in the art to which this disclosure belongs. All patents and publications referred to herein are incorporated by reference in their entireties.

### Definitions

As used herein, the terms “administer,” “administration” or “administering” refer to (1) providing, giving, dosing, and/or prescribing by either a health practitioner or his authorized agent or under his or her direction according to the disclosure; and/or (2) putting into, taking or consuming by the mammal, according to the disclosure.

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The terms “active pharmaceutical ingredient” and “drug” include, but are not limited to, the compounds described herein and, more specifically, compounds of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, and their features and limitations as described herein.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., increased sensitivity to apoptosis). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

The term “pharmaceutically acceptable salt” refers to salts derived from a variety of organic and inorganic counter ions known in the art. Pharmaceutically acceptable acid addition salts can be formed with inorganic acids and organic acids. Preferred inorganic acids from which salts can be derived include, for example, hydrochloric acid, hydrobromic acid, sulfuric acid, nitric acid and phosphoric acid. Preferred organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs disclosed herein, can also be incorporated into the described compositions and methods.

As used herein, the terms “treat,” “treatment,” and/or “treating” may refer to the management of a disease, disorder, or pathological condition, or symptom thereof with the intent to cure, ameliorate, stabilize, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition).

As used herein, the terms “modulate” and “modulation” refer to a change in biological activity for a biological molecule (e.g., a protein, gene, peptide, antibody, and the like), where such change may relate to an increase in biological activity (e.g., increased activity, agonism, activation, expression, upregulation, and/or increased expression) or decrease in biological activity (e.g., decreased activity, antagonism, suppression, deactivation, downregulation, and/or decreased expression) for the biological molecule.

As used herein, the term “prodrug” refers to a derivative of a compound described herein, the pharmacologic action of which results from the conversion by chemical or metabolic processes in vivo to the active compound. Prodrugs include compounds wherein an amino acid residue, or a polypeptide chain of two or more (e.g., two, three or four) amino acid residues is covalently joined through an amide or ester bond to a free amino, hydroxyl or carboxylic acid group of a compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004. The amino acid residues include but are not limited to the 20 naturally occurring amino acids commonly designated by one or three letter symbols but also include, for example, 4-hydroxyproline, hydroxylysine, desmosine, isodesmosine, 3-methylhistidine, beta-alanine, gamma-aminobutyric acid, citrulline, homocysteine, homoserine, ornithine and methionine sulfone.

Additional types of prodrugs are also encompassed. For instance, free carboxyl groups can be derivatized as amides or alkyl esters (e.g., methyl esters and acetoxy methyl esters). Prodrug esters as employed herein includes esters and carbonates formed by reacting one or more hydroxyls of compounds of the method of the disclosure with alkyl, alkoxy, or aryl substituted acylating agents employing procedures known to those skilled in the art to generate acetates, pivalates, methylcarbonates, benzoates and the like. As further examples, free hydroxyl groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxyl and amino groups are also included, as are carbonate prodrugs, sulfonate prodrugs, sulfonate esters and sulfate esters of hydroxyl groups. Free amines can also be derivatized to amides, sulfonamides or phosphonamides. All of the stated prodrug moieties may incorporate groups including but not limited to ether, amine and carboxylic acid functionalities. Moreover, any compound that can be converted in vivo to provide the bioactive agent (e.g., a compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004) is a prodrug within the scope of the disclosure. Various forms of prodrugs are well known in the art. A comprehensive description of pro drugs and prodrug derivatives are described in: (a) The Practice of Medicinal Chemistry, Camille G. Wermuth et al., (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds., (Harwood Academic Publishers, 1991). In general, prodrugs may be designed to improve the penetration of a drug across biological membranes in order to obtain improved drug absorption, to prolong duration of action of a drug (slow release of the parent drug from a prodrug, decreased first-pass metabolism of the drug), to target the drug action (e.g. organ or tumor-targeting, lymphocyte targeting), to modify or improve aqueous solubility of a drug (e.g., i.v. preparations and eyedrops), to improve topical drug delivery (e.g. dermal and ocular drug delivery), to improve the chemical/enzymatic stability of a drug, or to decrease off-target drug effects, and more generally in order to improve the therapeutic efficacy of the compounds utilized in the disclosure.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this disclosure.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

“Depressive symptoms” include low mood, diminished interest in activities, psychomotor slowing or agitation, changes in appetite, poor concentration or indecisiveness, excessive guilt or feelings of worthlessness, suicidal ideations, and/or suicidal actions may occur in the context of depressive disorders, bipolar disorders, mood disorders due to a general medical condition, substance-induced mood disorders, other unspecified mood disorders, and also may be present in association with a range of other psychiatric disorders, including but not limited to psychotic disorders, cognitive disorders, eating disorders, anxiety disorders and personality disorders. The longitudinal course of the disorder, the history, and type of symptoms, and etiologic factors help distinguish the various forms of mood disorders from each other.

“Depression symptoms rating scale” refers to any one of a number of standardized questionnaires, clinical instruments, or symptom inventories utilized to measure symptoms and symptom severity in depression. Such rating scales are often used in clinical studies to define treatment outcomes, based on changes from the study's entry point(s) to endpoint(s). Such depression symptoms rating scales include, but are not limited to, The Quick Inventory of Depressive-Symptomatology Self-Report (QIDS-SR₁₆), the 17-Item Hamilton Rating Scale of Depression (HRSD₁₇), the 30-Item Inventory of Depressive Symptomatology (IDS-C₃₀), or The Montgomery-Asperg Depression Rating Scale (MADRS). Such ratings scales may involve patient self-report or be clinician rated. A 50% or greater reduction in a depression ratings scale score over the course of a clinical trial (starting point to endpoint) is typically considered a favorable response for most depression symptoms rating scales. “Remission” in clinical studies of depression often refers to achieving at, or below, a particular numerical rating score on a depression symptoms rating scale (for instance, less than or equal to 7 on the HRSD₁₇; or less than or equal to 5 on the QIDS-SR₁₆; or less than or equal to 10 on the MADRS).

“Anxiety symptom rating scale” refers to any one of a number of standardized questionnaires, clinical instruments, or symptom inventories utilized to measure symptoms and symptom severity in anxiety. Such rating scales are often used in clinical studies to define treatment outcomes, based on changes from the study's entry point(s) to endpoint(s). Such anxiety symptoms rating scales include, but are not limited to, State-Trait Anxiety Inventory (STAI), the Hamilton Anxiety Rating Scale (HAM-A), the Beck Anxiety Inventory (BAT), and the Hospital Anxiety and Depression Scale-Anxiety (HADS-A). Such ratings scales may involve patient self-report or be clinician rated. A 50% or greater reduction in a depression or anxiety ratings scale score over the course of a clinical trial (starting point to endpoint) is typically considered a favorable response for most depression and anxiety symptoms rating scales. “Remission” in clinical studies of depression often refers to achieving at, or below, a particular numerical rating score on a depression symptoms rating scale (for instance, less than or equal to 39 on the STAI; or less than or equal to 9 on the BAI; or less than or equal to 7 on the HADS-A).

“Alkyl” refers to a straight or branched hydrocarbon chain radical consisting solely of carbon and hydrogen atoms, containing no unsaturation, having from one to ten carbon atoms (e.g., (C₁₋₁₀)alkyl or C₁₋₁₀ alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (Pr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocyclyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C₂₋₁₀)alkenyl or C₂₋₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Carboxaldehyde” refers to a —(C═O)H radical.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e. (C₃₋₁₀)cycloalkyl or C₃₋₁₀ cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(C═O)— attached through the carbonyl carbon wherein the alkoxy group has the indicated number of carbon atoms. Thus a (C₁₋₆)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)— and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═0)O— radical wherein R is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the R of an acyloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acylsulfonamide” refers a —S(O)₂—N(R^{a})—C(═O)— radical, where R^{a} is hydrogen, alkyl, fluoroalkyl, carbocyclyl, carbocyclylalkyl, aryl, aralkyl, heterocycloalkyl, heterocycloalkylalkyl, heteroaryl or heteroarylalkyl. Unless stated otherwise specifically in the specification, an acylsulfonamide group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{a}, and NR^{a}R^{a} each as described above. N-oxides can be prepared by treatment of the corresponding amino group with, for example, hydrogen peroxide or m-chloroperoxybenzoic acid.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R₂ of —N(R)₂ of the amide may optionally be taken together with the nitrogen to which it is attached to form a 4-, 5-, 6- or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd}Ed., John Wiley & Sons, New York, N.Y., 1999, which is incorporated herein by reference in its entirety.

“Aromatic” or “aryl” or “Ar” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “aryloxy” refers to the group —O-aryl.

The term “substituted aryloxy” refers to aryloxy wherein the aryl substituent is substituted (i.e., —O-(substituted aryl)). Unless stated otherwise specifically in the specification, the aryl moiety of an aryloxy group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively.

“Heteroalkylheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” or “Het” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e., thienyl). Unless stated otherwise specifically in the specification, a heteroaryl moiety is optionally substituted by one or more substituents which are independently: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” refers to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group may consist of 3 ring atoms, 4 ring atoms, etc., up to and including 18 ring atoms. Unless stated otherwise specifically in the specification, the heterocycloalkyl radical is a monocyclic, bicyclic, tricyclic or tetracyclic ring system, which may include fused or bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Nitro” refers to the —NO₂ radical.

“Oxa” refers to the —O— radical.

“Oxo” refers to the ═O radical.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In some embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or preferred enantiomers can be prepared by asymmetric syntheses. See, for example, Jacques, et al., Enantiomers, Racemates and Resolutions, Wiley Interscience, New York (1981); E. L. Eliel, Stereochemistry of Carbon Compounds, McGraw-Hill, New York (1962); and E. L. Eliel and S. H. Wilen, Stereochemistry of Organic Compounds, Wiley-Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, Third Edition, John Wiley & Sons, New York (1999).

“Solvate” refers to a compound in physical association with one or more molecules of a pharmaceutically acceptable solvent.

“Substituted” means that the referenced group may have attached one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S-(optionally substituted alkyl), —S-(optionally substituted aryl), —S-(optionally substituted heteroaryl) and —S-(optionally substituted heterocycloalkyl).

“Sulfinyl” refers to groups that include —S(O)—H, —S(O)-(optionally substituted alkyl), —S(O)-(optionally substituted amino), —S(O)-(optionally substituted aryl), —S(O)-(optionally substituted heteroaryl) and —S(O)-(optionally substituted heterocycloalkyl).

“Sulfonyl” refers to groups that include —S(O₂)—H, —S(O₂)-(optionally substituted alkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6- or 7-membered ring. A sulfonamido group is optionally substituted by one or more of the substituents described for alkyl, cycloalkyl, aryl, heteroaryl, respectively.

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively.

Compounds of the disclosure also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

For the avoidance of doubt, it is intended herein that particular features (for example integers, characteristics, values, uses, diseases, formulae, compounds or groups) described in conjunction with a particular aspect, embodiment or example of the disclosure are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The disclosure is not restricted to any details of any disclosed embodiments. The disclosure extends to any novel one, or novel combination, of the features disclosed in this specification (including any accompanying claims, abstract and drawings), or to any novel one, or any novel combination, of the steps of any method or process so disclosed.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the disclosure. All embodiments of the disclosure can, in the alternative, be more specifically defined by any of the transitional terms “comprising,” “consisting essentially of,” and “consisting of.”

(2,6)-hydroxynorketamine Compounds

In the current “ketamine paradigm”, ketamine and norketamine (NK) are considered to be responsible for the antinociceptive response in patients with complex regional pain syndrome (CRPS). However the routine use of the drug is hindered by unwanted central nervous system (CNS) effects. Approximately 30% of patients do not respond to ketamine treatment. Additionally, ketamine treatment is associated with serious side effects due to the drug's anesthetic properties and abuse potential.

Recent studies have demonstrated that in CRPS patients receiving a continuous 5-day infusion of (R,S)-ketamine the primary circulating metabolites were (R,S)-dehydronorketamine (DHNK) and (2S,6S;2R,6R)-hydroxynorketamine (HNK). The data suggest that downstream metabolites play a role in ketamine analgesic efficacy, although little is known about the metabolites' pharmacological activity.

Studies using sub-anesthetic does of (R,S)-Ketamine demonstrated that this drug is effective in the treatment of neuropathic and chronic pain, including the treatment of patients suffering from complex regional pain syndrome (CRPS). The analysis of the plasma samples obtained from CRPS patients receiving (R,S)-ketamine as a 5-day continuous infusion revealed that the primary drug, (R,S)-ketamine, was not primarily responsible for the therapeutic response. Active agents responsible for the therapeutic response to ketamine in patients are (2R,6R; 2S,6S)-hydroxynorketamine and (R,S)-dehydronorketamine, which are ketamine metabolites. These metabolites are produced by a number of liver enzymes identified as Cytochrome P450s (CYPs) including CYP2A6, CYP2B6, CYP2C9, CYP2D6, and CYP3A5.

The CYPs are polymorphic, which means that they are not equally active in all humans. Thus it is likely that ketamine's failure to elicit a therapeutic response in about 30% of treated patients is due the activity differences of one or more of the identified CYPs among patients leading to unequal production of (2R,6R,2S,6S)-hydroxynorketamine and/or (S)-dehydronorketamine.

In one aspect, the present disclosure provides compounds that can be administered directly to the patient in order to increase the response rate and to avoid treatment-limiting CNS side effects produced by (R,S)-ketamine. The CNS effects are associated with (R,S)-ketamine's activity at the NMDA receptor. (2R,6R; 2S,6S)-hydroxynorketamine (HNK), for example, is not active at the NMDA receptor and thus avoids these side effects. Direct administration of the (2S,6S)-hydroxynorketamine metabolite and/or DHNK has the advantage of producing a therapeutic response in a greater percentage of patients than ketamine (2S,6S)-hydroxynorketamine and DHNK also have a long plasma half-lives and are orally bioavailable. Thus, oral formulations for daily administration are included in the disclosure.

Ketamine rapidly (often within hours) exerts rapid antidepressant effects in patients suffering from depression following a single administration, which lasts for days and that can be sustained with repeated administration. A total of twelve HNK metabolites are formed from the metabolism of ketamine in vivo, with the (2,6)-HNKs being the most prevalent in the majority species studied. There is preclinical evidence to suggest that the (2,6)-HNKs play a critical role in mediating the sustained antidepressant-relevant actions of the parent compound ketamine. Namely, it was demonstrated that deuterium substitution at the C6 position of racemic ketamine, a chemical modification which specifically attenuates its metabolism to form the (2,6)-HNKs, prevents the sustained (observed 24 h post-treatment) antidepressant-relevant behavioral effects induced by ketamine in the mouse forced swim and learned helplessness tests. Likewise, the analogous modification to (R)-ketamine, which specifically attenuates its metabolism to (2R,6R)-HNK and (2R,6S)-HNK, suppressed its antidepressant-relevant behavioral effectiveness compared to that of unmodified (R)-ketamine, further supporting a role of (2R,6R)-HNK (the predominant HNK) and possibly (2R,6S)-HNK in at least partly mediating the antidepressant-relevant effects of ketamine. However, the contribution of the (2,6)-HNKs to mediating ketamine's actions has been called into question by some contrasting reports.

Several studies have provided evidence that (2R,6R)-hydroxynorketamine (HNK) and (2S,6S)-HNK induce antidepressant-relevant effects in a variety of preclinical rodent behavioral tests thought to predict antidepressant efficacy. However, the potential behavioral effects of other HNKs have not been studied to date.

In particular, it was reported that attenuating (R)-ketamine's metabolism to (2R,6R)- and (2R,6S)-HNK, either via deuterium substitution or prior administration with a CYP inhibitor cocktail, did not attenuate ketamine's antidepressant-like behavioral effects. Additionally, independent of their role in ketamine's antidepressant actions, a growing number of studies have demonstrated that direct administration of (2R,6R)-HNK and, to a lesser extent, (2S,6S)-HNK rapidly induces antidepressant-relevant effects in rodent studies, including behavioral effects used to predict antidepressant efficacy in a number of rodent tests. Several studies have also demonstrated antidepressant-relevant behavioral effects following (2S,6S)-HNK administration to, albeit with lower apparent effectiveness when compared to (2R,6R)-HNK under similar experimental conditions.

Consistent with the greater relative antidepressant effectiveness of (2R,6R)-HNK compared to (2S,6S)-HNK, it has also been reported that (2R,6R)-, but not (2S,6S)-HNK, exerts behavioral effects when tested at equivalent doses in rodent tests thought to predict antidepressant efficacy. However, several studies have demonstrated that (2S,6S)-HNK also exerts antidepressant-like behavioral effects, albeit with lower apparent effectiveness when compared to (2R,6R)-HNK under similar experimental conditions. One study has reported that (2S,6S)-, but not (2R,6R)-HNK, reduced forced swim test immobility time and reversed measures of anhedonia following chronic stress in mice. Although not wishing to be bound by theory, this result suggest that it is possible that additional HNK metabolites exert antidepressant-relevant behavioral actions with even greater effectiveness compared to (2R,6R)-HNK or (2S,6S)-HNK. As preclinical studies have shown that (2R,6R)-HNK lacks the dissociative properties and abuse potential of ketamine, identifying the structural characteristics that confer enhanced behavioral effectiveness may inform the development of novel drug candidates that have an improved safety profile.

The opiod epidemic costs the US government about 78 billion dollars annually. Greater than 2000 deaths from opioid use/overdose were seen in Maryland in 2018. Greater than 50% of of opioid addicts suffer from emotional impairment especially during long-term abstinence, and 60-85% relapse rate to opioid-seeking after long-term abstinence. Therefore, one strategy for preventing relapse is to alleviate long-term emotional impairment.

Scheme 1 below shows current treatments for opioid addiction; however, there is a need for novel, effective treatments lacking abuse potential properties.

Scheme 1: Current treatments for opioid addicion

| | Methadone | Buprenorphine | Naltrexone |
|---|---|---|---|
| Pharmacology | Partial MOPr agonist | Partial MOPr agonist | MOPr antagonist |
| | | KOPr antagonist | KOPr antagonist |
| | | Partial nociceptin | DOPr antagonist |
| | | receptor agonist | |
| Properties | High bioavailability | Less euphoric effects | No abuse potential |
| | (when administered | compared to methadone | Poor treatment retention |
| | orally) | Long-term safety? | |
| | Addiction potential | | |

Methods for treating opioid addiction include methadone, buprenorphine, naloxone, and naltrexone. Ketamine has shown promising effects for the treatment of drug addiction. In particular, subanaesthetic doses of ketamine reduced alcohol consumption in rodents, augmented the effects of benzodiazepines on the alleviation of alcohol withdrawal syndrome in humans, enhanced the motivation and decreased cue-induced craving to cocaine use and prolonged abstinence period in heroin addicts; however, studies on the effects of ketamine in the treatment of opioid addiction/abstinence/relapse have not yet been performed. Although ketamine has shown some potential in the treatment of drug addiction, its clinical use for the treatment of opioid addiction is limited due to its psychotomimetic properties and its abuse potential. Disclosed herein is data demonstrating that a hydroxynorketamine (HNK) metabolite of ketamine, the (2R,6R)-HNK, is sufficient to exert ketamine's antidepressant actions while lacking the side effects of ketamine, including abuse potential, which is a critical aspect for an ideal pharmacotherapy for drug addiction treatment. In one aspect, the disclosure provides compounds, such as (2R,6R)-HNK, having no addiction potential, and are useful as an effective pharmacotherapy for the treatment of opioid addiction and prevention of relapse during abstinence. In some embodiments, the compounds of the disclosure are useful in the prevention and treatment of opioid addiction and lack abuse liability or any dissociation side effects.

The disclosure provides a compound of formula (I), or a pharmaceutically acceptable salt, solvate, hydrate, cocrystal, or prodrug thereof:

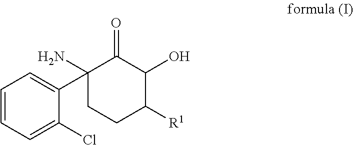

wherein in formula (I):
R¹ is independently selected at each occurrence from halogen, hydroxyl, amino, cyano, C₁-C₄alkyl, C₁-C₄alkoxy, mono- and di-C₁-C₄alkylamino, C₁-C₂haloalkyl, and C₁-C₂haloalkoxy.

In some embodiments, the compound of formula (I) is a compound of any one of formula (Ia), formula (Ib), formula (Ic), or formula (Id), formula (Ie), formula (If), formula (Ig), or formula (Ih), or a pharmaceutically acceptable salt, solvate, hydrate, cocrystal, or prodrug thereof:

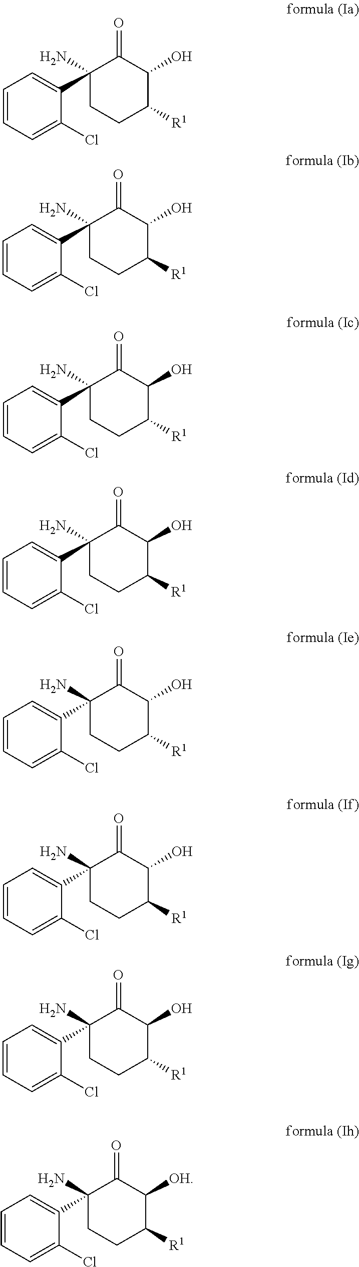

In some embodiments, the compound of formula (I) is a compound of any one of formula (Ia), formula (Ib), formula (Ic), or formula (Id), or a pharmaceutically acceptable salt, solvate, hydrate, cocrystal, or prodrug thereof:

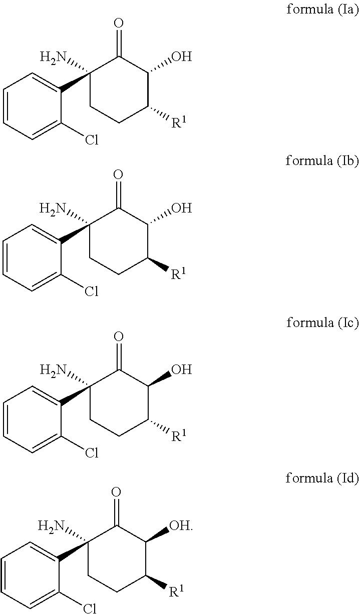

In some embodiments, R¹ is independently selected at each occurrence from C₁-C₄alkyl. In some embodiments, the C₁-C₄alkyl is substituted. In some embodiments, the C₁-C₄alkyl is unsubstituted. In some embodiments, R¹ is methyl. In some embodiments, the methyl is substituted. In some embodiments, the methyl is unsubstituted.

The disclosure also provides a compound of formula (II), or a pharmaceutically acceptable salt, solvate, hydrate, cocrystal, or prodrug thereof:

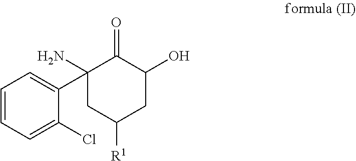

wherein in formula (II):
R¹ is independently selected at each occurrence from halogen, hydroxyl, amino, cyano, C₁-C₄alkyl, C₁-C₄alkoxy, mono- and di-C₁-C₄alkylamino, C₁-C₂haloalkyl, and C₁-C₂haloalkoxy.

In some embodiments, the compound of formula (II) is a compound of any one of formula (IIa), formula (IIb), formula (IIc), or formula (IId), formula (IIe), formula (IIf), formula (IIg), or formula (IIh), or a pharmaceutically acceptable salt, solvate, hydrate, cocrystal, or prodrug thereof:

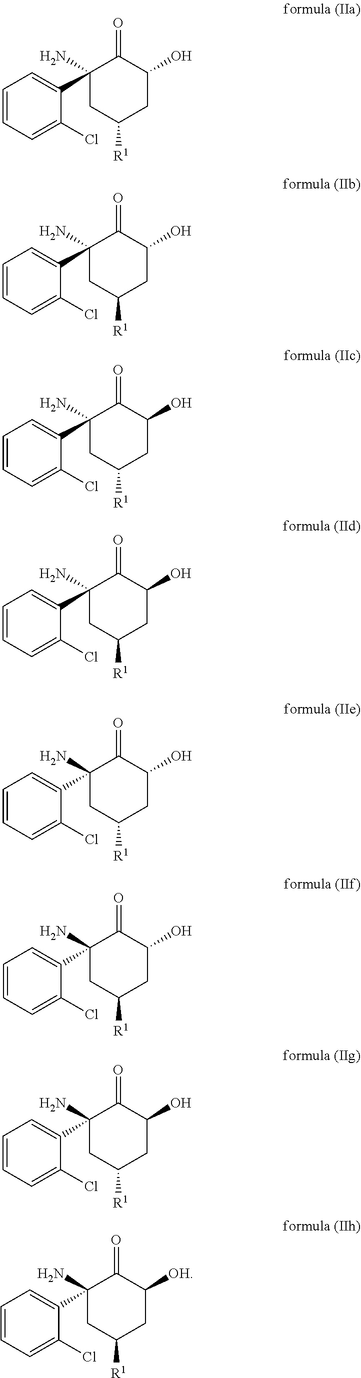

In some embodiments, the compound of formula (II) is a compound of any one of formula (IIa), formula (IIb), formula (IIc), or formula (IId), or a pharmaceutically acceptable salt, solvate, hydrate, cocrystal, or prodrug thereof:

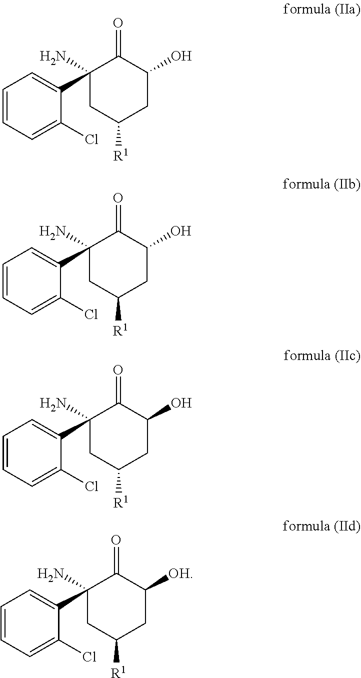

In some embodiments, R¹ is independently selected at each occurrence from C₁-C₄alkyl. In some embodiments, the C₁-C₄alkyl is substituted. In some embodiments, the C₁-C₄alkyl is unsubstituted. In some embodiments, R¹ is methyl. In some embodiments, the methyl is substituted. In some embodiments, the methyl is unsubstituted.

The disclosure also provides a compound of formula (III), or a pharmaceutically acceptable salt, solvate, hydrate, cocrystal, or prodrug thereof:

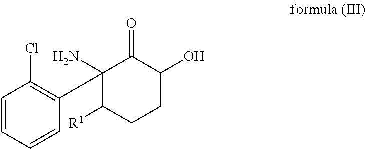

wherein in formula (III):
R¹ is independently selected at each occurrence from halogen, hydroxyl, amino, cyano, C₁-C₄alkyl, C₁-C₄alkoxy, mono- and di-C₁-C₄alkylamino, C₁-C₂haloalkyl, and C₁-C₂haloalkoxy.

In some embodiments, the compound of formula (III) is a compound of any one of formula (IIIa), formula (IIIb), formula (IIIc), or formula (IIId), formula (IIIe), formula (IIIf), formula (IIIg), formula (IIIh), or a pharmaceutically acceptable salt, solvate, hydrate, cocrystal, or prodrug thereof:

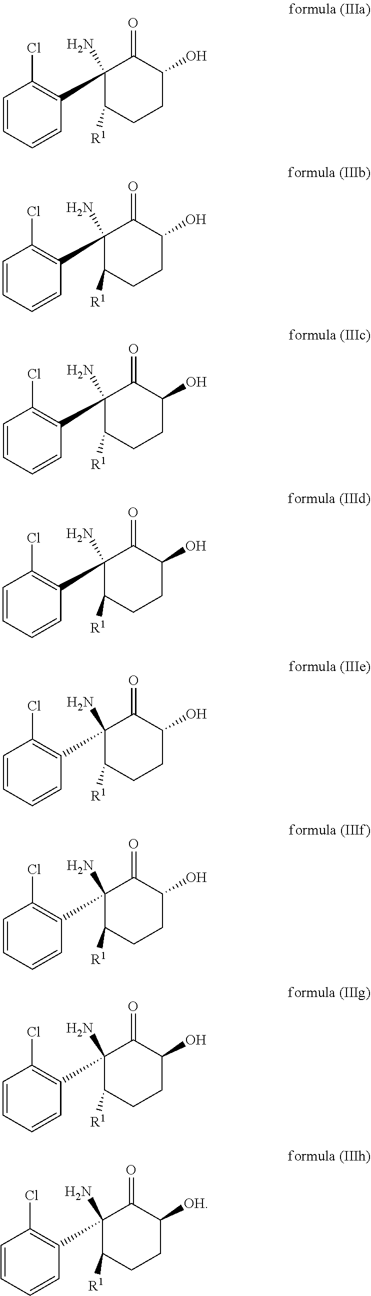

In some embodiments, the compound of formula (III) is a compound of any one of formula (IIIa), formula (IIIb), formula (IIIc), or formula (IIId), or a pharmaceutically acceptable salt, solvate, hydrate, cocrystal, or prodrug thereof:

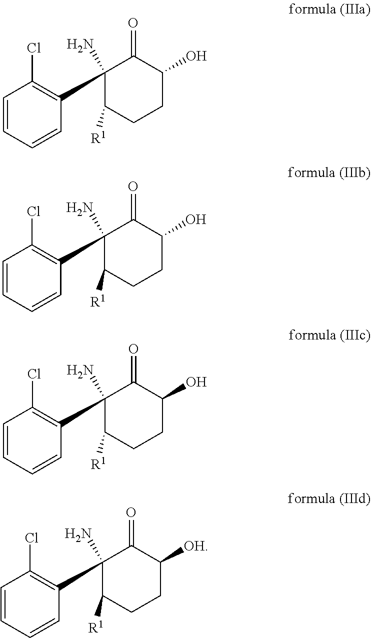

In some embodiments, R¹ is independently selected at each occurrence from C₁-C₄alkyl. In some embodiments, the C₁-C₄alkyl is substituted. In some embodiments, the C₁-C₄alkyl is unsubstituted. In some embodiments, R¹ is methyl. In some embodiments, the methyl is substituted. In some embodiments, the methyl is unsubstituted.

In some embodiments, the compound is of any one of formulas 1001-1168, or a pharmaceutically acceptable salt, solvate, hydrate, cocrystal, or prodrug thereof:

| 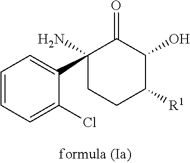 | |
|---|---|
| Compound # | R¹ |
| 1001 | —CH₃ |
| 1002 | —CH₂CH₃ |
| 1003 | —CH₂CH₂CH₃ |
| 1004 | —(CH)(CH₃)₂ |
| 1005 | —CH₂CH₂CH₂CH₃ |
| 1006 | —CH₂(CH)(CH₃)₂ |
| 1007 | —(CH₃)₃ |
| 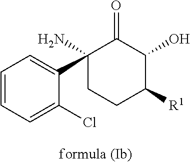 | |
| Compound # | R¹ |
| 1008 | —CH₃ |
| 1009 | —CH₂CH₃ |
| 1010 | —CH₂CH₂CH₃ |
| 1011 | —(CH)(CH₃)₂ |
| 1012 | —CH₂CH₂CH₂CH₃ |
| 1013 | —CH₂(CH)(CH₃)₂ |
| 1014 | —(CH₃)₃ |
| 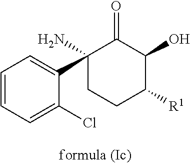 | |
| Compound # | R¹ |
| 1015 | —CH₃ |
| 1016 | —CH₂CH₃ |
| 1017 | —CH₂CH₂CH₃ |
| 1018 | —(CH)(CH₃)₂ |
| 1019 | —CH₂CH₂CH₂CH₃ |
| 1020 | —CH₂(CH)(CH₃)₂ |
| 1021 | —(CH₃)₃ |
| 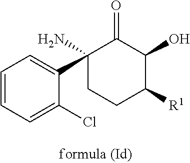 | |
| Compound # | R¹ |
| 1022 | —CH₃ |
| 1023 | —CH₂CH₃ |
| 1024 | —CH₂CH₂CH₃ |
| 1025 | —(CH)(CH₃)₂ |
| 1026 | —CH₂CH₂CH₂CH₃ |
| 1027 | —CH₂(CH)(CH₃)₂ |
| 1028 | —(CH₃)₃ |
| 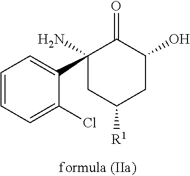 | |
| Compound # | R¹ |
| 1029 | —CH₃ |
| 1030 | —CH₂CH₃ |
| 1031 | —CH₂CH₂CH₃ |
| 1032 | —(CH)(CH₃)₂ |
| 1033 | —CH₂CH₂CH₂CH₃ |
| 1034 | —CH₂(CH)(CH₃)₂ |
| 1035 | —(CH₃)₃ |
| 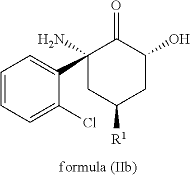 | |
| Compound # | R¹ |
| 1036 | —CH₃ |
| 1037 | —CH₂CH₃ |
| 1038 | —CH₂CH₂CH₃ |
| 1039 | —(CH)(CH₃)₂ |
| 1040 | —CH₂CH₂CH₂CH₃ |
| 1041 | —CH₂(CH)(CH₃)₂ |
| 1042 | —(CH₃)₃ |
| 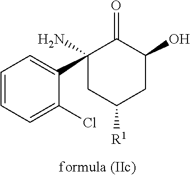 | |
| Compound # | R¹ |
| 1043 | —CH₃ |
| 1044 | —CH₂CH₃ |
| 1045 | —CH₂CH₂CH₃ |
| 1046 | —(CH)(CH₃)₂ |
| 1047 | —CH₂CH₂CH₂CH₃ |
| 1048 | —CH₂(CH)(CH₃)₂ |
| 1049 | —(CH₃)₃ |
| 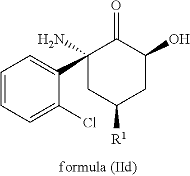 | |
| Compound # | R¹ |
| 1050 | —CH₃ |
| 1051 | —CH₂CH₃ |
| 1052 | —CH₂CH₂CH₃ |
| 1053 | —(CH)(CH₃)₂ |
| 1054 | —CH₂CH₂CH₂CH₃ |
| 1055 | —CH₂(CH)(CH₃)₂ |
| 1056 | —(CH₃)₃ |
| 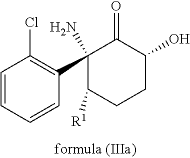 | |
| Compound # | R¹ |
| 1057 | —CH₃ |
| 1058 | —CH₂CH₃ |
| 1059 | —CH₂CH₂CH₃ |
| 1060 | —(CH)(CH₃)₂ |
| 1061 | —CH₂CH₂CH₂CH₃ |
| 1062 | —CH₂(CH)(CH₃)₂ |
| 1063 | —(CH₃)₃ |
| 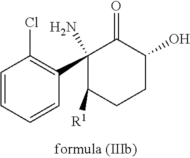 | |
| Compound # | R¹ |
| 1064 | —CH₃ |
| 1065 | —CH₂CH₃ |
| 1066 | —CH₂CH₂CH₃ |
| 1067 | —(CH)(CH₃)₂ |
| 1068 | —CH₂CH₂CH₂CH₃ |
| 1069 | —CH₂(CH)(CH₃)₂ |
| 1070 | —(CH₃)₃ |
| 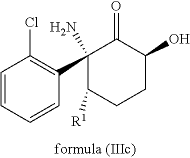 | |
| Compound # | R¹ |
| 1071 | —CH₃ |
| 1072 | —CH₂CH₃ |
| 1073 | —CH₂CH₂CH₃ |
| 1074 | —(CH)(CH₃)₂ |
| 1075 | —CH₂CH₂CH₂CH₃ |
| 1076 | —CH₂(CH)(CH₃)₂ |
| 1077 | —(CH₃)₃ |
| 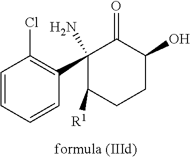 | |
| Compound # | R¹ |
| 1078 | —CH₃ |
| 1079 | —CH₂CH₃ |
| 1080 | —CH₂CH₂CH₃ |
| 1081 | —(CH)(CH₃)₂ |
| 1082 | —CH₂CH₂CH₂CH₃ |
| 1083 | —CH₂(CH)(CH₃)₂ |
| 1084 | —(CH₃)₃ |
| 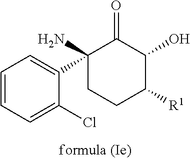 | |
| Compound # | R¹ |
| 1085 | —CH₃ |
| 1086 | —CH₂CH₃ |
| 1087 | —CH₂CH₂CH₃ |
| 1088 | —(CH)(CH₃)₂ |
| 1089 | —CH₂CH₂CH₂CH₃ |
| 1090 | —CH₂(CH)(CH₃)₂ |
| 1091 | —(CH₃)₃ |
| 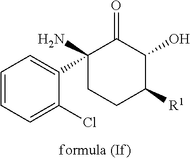 | |
| Compound # | R¹ |
| 1092 | —CH₃ |
| 1093 | —CH₂CH₃ |
| 1094 | —CH₂CH₂CH₃ |
| 1095 | —(CH)(CH₃)₂ |
| 1096 | —CH₂CH₂CH₂CH₃ |
| 1097 | —CH₂(CH)(CH₃)₂ |
| 1098 | —(CH₃)₃ |
| 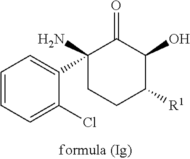 | |
| Compound # | R¹ |
| 1099 | —CH₃ |
| 1100 | —CH₂CH₃ |
| 1101 | —CH₂CH₂CH₃ |
| 1102 | —(CH)(CH₃)₂ |
| 1103 | —CH₂CH₂CH₂CH₃ |
| 1104 | —CH₂(CH)(CH₃)₂ |
| 1105 | —(CH₃)₃ |
| 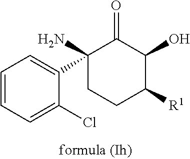 | |
| Compound # | R¹ |
| 1106 | —CH₃ |
| 1107 | —CH₂CH₃ |
| 1108 | —CH₂CH₂CH₃ |
| 1109 | —(CH)(CH₃)₂ |
| 1110 | —CH₂CH₂CH₂CH₃ |
| 1111 | —CH₂(CH)(CH₃)₂ |
| 1112 | —(CH₃)₃ |
| 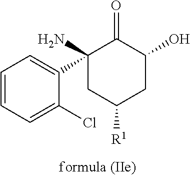 | |
| Compound # | R¹ |
| 1113 | —CH₃ |
| 1114 | —CH₂CH₃ |
| 1115 | —CH₂CH₂CH₃ |
| 1116 | —(CH)(CH₃)₂ |
| 1117 | —CH₂CH₂CH₂CH₃ |
| 1118 | —CH₂(CH)(CH₃)₂ |
| 1119 | —(CH₃)₃ |
| 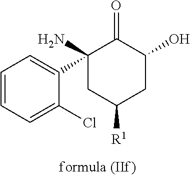 | |
| Compound # | R¹ |
| 1120 | —CH₃ |
| 1121 | —CH₂CH₃ |
| 1122 | —CH₂CH₂CH₃ |
| 1123 | —(CH)(CH₃)₂ |
| 1124 | —CH₂CH₂CH₂CH₃ |
| 1125 | —CH₂(CH)(CH₃)₂ |
| 1126 | —(CH₃)₃ |
| 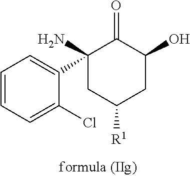 | |
| Compound # | R¹ |
| 1127 | —CH₃ |
| 1128 | —CH₂CH₃ |
| 1129 | —CH₂CH₂CH₃ |
| 1130 | —(CH)(CH₃)₂ |
| 1131 | —CH₂CH₂CH₂CH₃ |
| 1132 | —CH₂(CH)(CH₃)₂ |
| 1133 | —(CH₃)₃ |
| 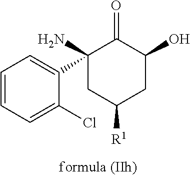 | |
| Compound # | R¹ |
| 1134 | —CH₃ |
| 1135 | —CH₂CH₃ |
| 1136 | —CH₂CH₂CH₃ |
| 1137 | —(CH)(CH₃)₂ |
| 1138 | —CH₂CH₂CH₂CH₃ |
| 1139 | —CH₂(CH)(CH₃)₂ |
| 1140 | —(CH₃)₃ |
| 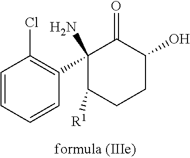 | |
| Compound # | R¹ |
| 1141 | —CH₃ |
| 1142 | —CH₂CH₃ |
| 1143 | —CH₂CH₂CH₃ |
| 1144 | —(CH)(CH₃)₂ |
| 1145 | —CH₂CH₂CH₂CH₃ |
| 1146 | —CH₂(CH)(CH₃)₂ |
| 1147 | —(CH₃)₃ |
| 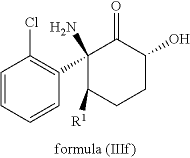 | |
| Compound # | R¹ |
| 1148 | —CH₃ |
| 1149 | —CH₂CH₃ |
| 1150 | —CH₂CH₂CH₃ |
| 1151 | —(CH)(CH₃)₂ |
| 1152 | —CH₂CH₂CH₂CH₃ |
| 1153 | —CH₂(CH)(CH₃)₂ |
| 1154 | —(CH₃)₃ |
| 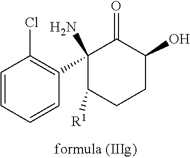 | |
| Compound # | R¹ |
| 1155 | —CH₃ |
| 1156 | —CH₂CH₃ |
| 1157 | —CH₂CH₂CH₃ |
| 1158 | —(CH)(CH₃)₂ |
| 1159 | —CH₂CH₂CH₂CH₃ |
| 1160 | —CH₂(CH)(CH₃)₂ |
| 1161 | —(CH₃)₃ |
| 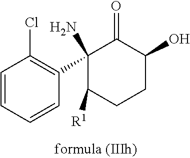 | |
| Compound # | R¹ |
| 1162 | —CH₃ |
| 1163 | —CH₂CH₃ |
| 1164 | —CH₂CH₂CH₃ |
| 1165 | —(CH)(CH₃)₂ |
| 1166 | —CH₂CH₂CH₂CH₃ |
| 1167 | —CH₂(CH)(CH₃)₂ |
| 1168 | —(CH₃)₃ |

In some embodiments, the compound is of any one of formulas 1001-1084, or a pharmaceutically acceptable salt, solvate, hydrate, cocrystal, or prodrug thereof:

In some embodiments, the compound is compound 1001, or a pharmaceutically acceptable salt, solvate, hydrate, cocrystal, or prodrug thereof:

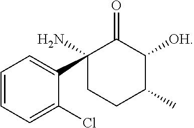 [SMILES: C[C@@H]1CC[C@@](N)(c2ccccc2Cl)C(=O)[C@@H]1O]

The disclosure also provides a compound of any one of formulas 2001 to 2004, or a pharmaceutically acceptable salt, solvate, hydrate, cocrystal, or prodrug thereof:

| Compound # | R¹ |
|---|---|
| 2001 | 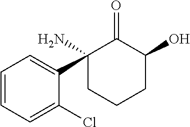 |
| 2002 | 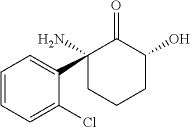 |
| 2003 | 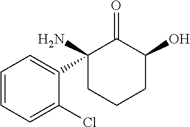 |
| 2004 | 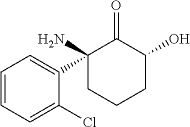 |

In some embodiments, the compound is a compound of formula 2001, or a pharmaceutically acceptable salt, solvate, hydrate, cocrystal, or prodrug thereof.

In some embodiments, the compound is a compound of formula 2002, or a pharmaceutically acceptable salt, solvate, hydrate, cocrystal, or prodrug thereof.

### Methods of Treatment

The compounds and compositions described herein can be used in methods for treating or preventing conditions and diseases, including but not limited to: a method of treating or preventing a depressive disorder in a patient in need of said treatment, the method comprising administering to the patient a therapeutically effective amount of a compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salts, solvates, hydrates, cocrystals, or prodrugs thereof; a method of treating or preventing an anxiety disorder in a patient in need of said treatment, the method comprising administering to the patient a therapeutically effective amount of a compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salts, solvates, hydrates, cocrystals, or prodrugs thereof; a method of treating or preventing drug addiction in a patient in need of said treatment, the method comprising administering to the patient a therapeutically effective amount of a compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salts, solvates, hydrates, cocrystals, or prodrugs thereof, a method of treating or preventing schizophrenia in a patient in need of said treatment, the method comprising administering to the patient a therapeutically effective amount of a compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salts, solvates, hydrates, cocrystals, or prodrugs thereof; a method of treating or preventing Alzheimer's dementia in a patient in need of said treatment, the method comprising administering to the patient a therapeutically effective amount of a compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salts, solvates, hydrates, cocrystals, or prodrugs thereof; a method of treating or preventing amyotrophic lateral sclerosis in a patient in need of said treatment, the method comprising administering to the patient a therapeutically effective amount of a compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salts, solvates, hydrates, cocrystals, or prodrugs thereof; a method of treating or preventing complex regional pain syndrome (CRPS) in a patient in need of said treatment, the method comprising administering to the patient a therapeutically effective amount of a compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (JIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salts, solvates, hydrates, cocrystals, or prodrugs thereof, a method of treating or preventing chronic pain in a patient in need of said treatment, the method comprising administering to the patient a therapeutically effective amount of a compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salts, solvates, hydrates, cocrystals, or prodrugs thereof, a method of treating or preventing neuropathic pain in a patient in need of said treatment, the method comprising administering to the patient a therapeutically effective amount of a compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salts, solvates, hydrates, cocrystals, or prodrugs thereof, a method of treating or preventing anhedonia in a patient in need of said treatment, the method comprising administering to the patient a therapeutically effective amount of a compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salts, solvates, hydrates, cocrystals, or prodrugs thereof, a method of treating or preventing fatigue in a patient in need of said treatment, the method comprising administering to the patient a therapeutically effective amount of a compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salts, solvates, hydrates, cocrystals, or prodrugs thereof.

Non-limiting examples of a depressive disorder include major depressive disorder, bipolar disorder, postpartum depression, drug withdrawal induced depression, treatment refractory depression, persistent depressive disorder (dysthymia), perinatal depression, seasonal affective disorder, seasonal depression, premenstrual dysphoric disorder, psychotic depression, suicidal ideation, suicidal actions, disruptive mood dysregulation disorder, premenstrual dysphoric disorder, substance/medication-induced depressive disorder, depressive disorder due to another medical condition, other specified depressive disorder, and an unspecified depressive disorder.

Non-limiting examples of anxiety disorders include general anxiety disorder, obsessive-compulsive disorder (OCD), post-traumatic stress disorder (PTSD), separation anxiety disorder, selective mutism, specific phobia, social anxiety disorder (social phobia), panic disorder, panic attack (specifier), agoraphobia, substance/medication-induced anxiety disorder, anxiety disorder due to another medical, other specified anxiety disorder, anhedonia, and unspecified anxiety disorder.

Non-limiting examples of drug addiction include nicotine addiction, alcohol addiction, cannabis addiction, cocaine addiction, and opioid addiction.

The disclosure includes a method of treating or preventing bipolar depression and major depressive disorder where an effective amount of the compound is an amount effective to decrease depressive symptoms, wherein a decrease in depressive symptoms is the achievement of a 50% or greater reduction of symptoms identified on a depression symptom rating scale, or a score less than or equal to 7 on the HRSD₁₇, or less than or equal to 5 on the QID-SR₁₆, or less than or equal to 10 on the MADRS.

The disclosure provides an amount effective to decrease painful symptoms; wherein a decrease in painful symptom is the achievement of a 50% or greater reduction of painful symptoms on a pain rating scale.

The disclosure provides a method of treating or preventing one or more of a depressive disorder, including major depressive disorder, bipolar disorder, postpartum depression, drug withdrawal induced depression, treatment refractory depression, persistent depressive disorder (dysthymia), perinatal depression, seasonal affective disorder, seasonal depression, premenstrual dysphoric disorder, psychotic depression, suicidal ideation, suicidal actions, disruptive mood dysregulation disorder, premenstrual dysphoric disorder, substance/medication-induced depressive disorder, depressive disorder due to another medical condition, other specified depressive disorder, and an unspecified depressive disorder, wherein a decrease in depressive symptoms is the achievement of a 50% or greater reduction of symptoms identified on a depression symptom rating scale, or a score less than or equal to 7 on the HRSD₁₇, or less than or equal to 5 on the QID-SR₁₆, or less than or equal to 10 on the MADRS.

The disclosure provides a method of treating or preventing one or more anxiety disorders, general anxiety disorder, obsessive-compulsive disorder (OCD), post-traumatic stress disorder (PTSD), separation anxiety disorder, selective mutism, specific phobia, social anxiety disorder (social phobia), panic disorder, panic attack (specifier), agoraphobia, substance/medication-induced anxiety disorder, anxiety disorder due to another medical, other specified anxiety disorder, anhedonia, and unspecified anxiety disorder, wherein an effective amount is an amount effective to decrease anxiety symptoms; wherein a decrease in anxiety symptoms is the achievement of a 50% or greater reduction of anxiety symptoms on an anxiety symptom rating scale, or a score less than or equal to 39 on the STAT, or less than or equal to 9 on the BAT, or less than or equal to 7 on the HADS-A.

In some embodiments, the methods of the invention further includes administering to the patient psychotherapy, talk therapy, cognitive behavioral therapy, exposure therapy, systematic desensitization, mindfulness, dialectical behavior therapy, interpersonal therapy, eye movement desensitization and reprocessing, social rhythm therapy, acceptance and commitment therapy, family-focused therapy, psychodynamic therapy, light therapy, computer therapy, cognitive remediation, exercise, or other types of therapy.

In some aspects, the disclosure provides a method of treating or preventing opioid addiction in a patient in need thereof. In some embodiments, the method comprises administering to the patient a therapeutically effective amount of a compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or a pharmaceutically acceptable salt, solvate, hydrate, cocrystal, or prodrug thereof. In some embodiments, the method comprises administering to the patient a therapeutically effective amount of (2R,6R)-hydroxynorketamine (HNK) (formula 2002), or a pharmaceutically acceptable salt, solvate, hydrate, cocrystal, or prodrug thereof. In some embodiments, the opioid addiction is further associated with a depressive disorder and/or an anxiety disorder. In some embodiments, the depressive disorder is selected from major depressive disorder, bipolar disorder, postpartum depression, drug withdrawal induced depression, treatment refractory depression, persistent depressive disorder (dysthymia), perinatal depression, seasonal affective disorder, seasonal depression, premenstrual dysphoric disorder, psychotic depression, suicidal ideation, suicidal actions, disruptive mood dysregulation disorder, premenstrual dysphoric disorder, substance/medication-induced depressive disorder, depressive disorder due to another medical condition, other specified depressive disorder, and an unspecified depressive disorder. In some embodiments, the anxiety disorder is selected from general anxiety disorder, obsessive-compulsive disorder (OCD), post-traumatic stress disorder (PTSD), separation anxiety disorder, selective mutism, specific phobia, social anxiety disorder (social phobia), panic disorder, panic attack (specifier), agoraphobia, substance/medication-induced anxiety disorder, anxiety disorder due to another medical, other specified anxiety disorder, anhedonia, and unspecified anxiety disorder.

In one aspect, the disclosure provides a method of treating or preventing opioid withdrawal or one or more symptoms associated with opioid withdrawal. In some embodiments, the method comprises administering to the patient a therapeutically effective amount of a compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or a pharmaceutically acceptable salt, solvate, hydrate, cocrystal, or prodrug thereof. In some embodiments, the method comprises administering to the patient a therapeutically effective amount of (2R,6R)-hydroxynorketamine (HNK) (formula 2002), or a pharmaceutically acceptable salt, solvate, hydrate, cocrystal, or prodrug thereof. In some embodiments, the one or more symptoms of opioid withdrawal is selected from dysphoria, anxiety, restlessness, irritability, insomnia, yawning, hallucinations, tremors, shaking, hot or cold flashes, goosebumps, sneezing, sweating, rapid breathing, elevated heart rate, elevated blood pressure, pupillary dilation, piloerection, headaches, body aches, muscle cramps, muscle aches, bone aches, joint aches, hyperalgesia, hyperkatifiteia, watery discharge from eyes and nose (lacrimation and rhinorrhea), nausea, vomiting, diarrhea, abdominal pain, anorexia and fever. In some embodiments, the opioid withdrawal is induced by administration of one or more opioid antagonists or partial agonists. In some embodiments, the opioid antagonist is naltrexone In some embodiments, the compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or a pharmaceutically acceptable salt, solvate, hydrate, cocrystal, or prodrug is administered concurrently, before, or after administration of the opioid antagonist or partial agonist. In some embodiments, the opioid partial agonist is selected from morphine, methadone, fentanyl, sufentanil and heroin. In some embodiments, the (2R,6R)-hydroxynorketamine (HNK) (formula 2002), or a pharmaceutically acceptable salt, solvate, hydrate, cocrystal, or prodrug is administered concurrently, before, or after administration of the opioid antagonist or partial agonist.

In one aspect, the disclosure provides a method of treating or preventing relapse of opioid addiction. In some embodiments, the method comprises administering to the patient a therapeutically effective amount of a compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or a pharmaceutically acceptable salt, solvate, hydrate, cocrystal, or prodrug thereof. In some embodiments, the method comprises administering to the patient a therapeutically effective amount of (2R,6R)-hydroxynorketamine (HNK) (formula 2002), or a pharmaceutically acceptable salt, solvate, hydrate, cocrystal, or prodrug thereof. In some embodiments, the patient previously reduced or eliminated use of one or more opioids in response to treatment with an effective amount of an anti-addiction treatment, and is no longer exposed to an effective amount of the anti-addiction treatment. In some embodiments, the patient has undergone physical and/or physiological withdrawal from one or more opioids. In some embodiments, the relapse is stress-induced. In some embodiments, the patient has undergone physiological withdrawal from the one or more opioids during the period of abstinence from, or limited or reduced use of, the one or more opioids. In some embodiments, the patient is no longer exposed to an effective amount of the anti-addiction treatment because the patient has become conditioned to the anti-addiction treatment. In some embodiments, the patient is no longer exposed to an effective amount of the anti-addiction treatment because the patient has reduced or eliminated exposure to the anti-addiction treatment.

In some embodiments, the opioid addiction comprises and addiction of one or more opioids selected from oxycodone, morphine, buprenorphine, codeine, fentanyl, opium, methadone, heroin, hydrocodone, hydromorphone, oxymorphone, meperidine, tramadol, propoxyphene, diphenoxylate, loperamide, nalbuphine, butorphanol, pentazocine, carfentanil and other fentanyl analogues, and combinations thereof. In some embodiments, the compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or a pharmaceutically acceptable salt, solvate, hydrate, cocrystal, or prodrug thereof is administered in a single dose. In some embodiments, the (2R,6R)-hydroxynorketamine (HNK) (formula 2002), or a pharmaceutically acceptable salt, solvate, hydrate, cocrystal, or prodrug thereof is administered in a single dose.

In some embodiments, the therapeutically effective amount of the compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or a pharmaceutically acceptable salt, solvate, hydrate, cocrystal, or prodrug thereof is between about 5 mg/kg to about 10 mg/kg. In some embodiments, the therapeutically effective amount of (2R,6R)-hydroxynorketamine (HNK) (formula 2002), or a pharmaceutically acceptable salt, solvate, hydrate, cocrystal, or prodrug thereof is between about 5 mg/kg to about 10 mg/kg.

In some embodiments, the therapeutically effective amount of the compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or a pharmaceutically acceptable salt, solvate, hydrate, cocrystal, or prodrug thereof is between about 5 mg/kg to about 10 mg/kg. In some embodiments, the therapeutically effective amount of (2R,6R)-hydroxynorketamine (HNK) (formula 2002), or a pharmaceutically acceptable salt, solvate, hydrate, cocrystal, or prodrug thereof is between about 5 mg/kg to about 10 mg/kg.

In some embodiments, the therapeutically effective amount of the compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or a pharmaceutically acceptable salt, solvate, hydrate, cocrystal, or prodrug thereof is about 10 mg/kg. In some embodiments, the therapeutically effective amount of (2R,6R)-hydroxynorketamine (HNK) or an analogue thereof, or a pharmaceutically acceptable salt, solvate, hydrate, cocrystal, or prodrug thereof is about 10 mg/kg.

In some embodiments, the prodrug is (R)-ketamine.

In one aspect, the disclosure provides the use of the compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or a pharmaceutically acceptable salt, solvate, hydrate, cocrystal, or prodrug thereof for treating or preventing opioid addiction in a patient in need thereof.

In one aspect, the disclosure provides the use of (2R,6R)-hydroxynorketamine (HNK) (formula 2002), or a pharmaceutically acceptable salt, solvate, hydrate, cocrystal, or prodrug thereof for treating or preventing opioid addiction in a patient in need thereof.

In one aspect, the disclosure provides the use of the compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or a pharmaceutically acceptable salt, solvate, hydrate, cocrystal, or prodrug thereof for treating or preventing opioid withdrawal or one or more symptoms associated with opioid withdrawal.

In one aspect, the disclosure provides the use of (2R,6R)-hydroxynorketamine (HNK) (formula 2002), or a pharmaceutically acceptable salt, solvate, hydrate, cocrystal, or prodrug thereof for treating or preventing opioid withdrawal or one or more symptoms associated with opioid withdrawal.

In one aspect, the disclosure provides the use of the compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or a pharmaceutically acceptable salt, solvate, hydrate, cocrystal, or prodrug thereof for treating or preventing relapse of opioid addiction.

In one aspect, the disclosure provides the use of (2R,6R)-hydroxynorketamine (HNK) (formula 2002), or a pharmaceutically acceptable salt, solvate, hydrate, cocrystal, or prodrug thereof for treating or preventing relapse of opioid addiction.

In some embodiments, the methods of treatment include providing certain dosage amounts of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salts, solvates, hydrates, cocrystals, or prodrugs thereof to a patient. In some embodiments, the dosage levels of each active agent of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single unit dosage form will vary depending upon the patient treated and the particular mode of administration.

In some embodiments a therapeutically effect amount is an amount that provide a plasma Cmax of a compound of Formula I of about of 0.25 mcg/mL to about 125 mcg/mL, or about 1 mcg/mL to about 50 mcg/mL. For peripheral indications formulations and methods that provide a Cmax of about 0.25 mcg/mL to about 25 mcg/mL are preferred, while for CNS indications, formulations and methods that provide a plasma Cmax of about 0.25 mcg/mL to about 125 mcg/mL are preferred. The disclosure also includes IV pharmaceutical compositions that provide about 0.2 mg to about 500 mg per dose of a compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salts, solvates, hydrates, cocrystals, or prodrugs thereof, In some embodiments, for peripheral indications, the pharmaceutical composition provides about 0.5 mg to about 500 mg/dose.

### Pharmaceutical Compositions

In an embodiment, the disclosure provides a pharmaceutical composition for use in the treatment of the diseases and conditions described herein.

The pharmaceutical compositions are typically formulated to provide a therapeutically effective amount of a compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salts, solvates, hydrates, cocrystals, or prodrugs thereof, as described herein, as the active ingredient. Typically, the pharmaceutical compositions also comprise one or more pharmaceutically acceptable excipients, carriers, including inert solid diluents and fillers, diluents, including sterile aqueous solution and various organic solvents, permeation enhancers, solubilizers and adjuvants.

The pharmaceutical compositions described above are for use in the treatment or prevention of, without limitation, a disease or condition selected from a depressive disorder, an anxiety disorder, drug addiction, schizophrenia, Alzheimer's dementia, amyotrophic lateral sclerosis, complex regional pain syndrome (CRPS), chronic pain, neuropathic pain, anhedonia, and fatigue, the pharmaceutical composition comprising one or more compounds, or pharmaceutically acceptable salts, solvates, hydrates, cocrystals, or prodrugs thereof, having any one of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, and a pharmaceutically acceptable carrier. In some embodiments, the concentration of a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, provided in the pharmaceutical compositions of the disclosure is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of a compound of any of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, provided in the pharmaceutical compositions of the disclosure is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, provided in the pharmaceutical compositions of the disclosure is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, provided in the pharmaceutical compositions of the disclosure is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, provided in the pharmaceutical compositions of the disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, provided in the pharmaceutical compositions of the disclosure is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5 g, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

Each of the compounds provided according to the disclosure is effective over a wide dosage range. For example, in the treatment of adult humans, dosages independently ranging from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician.

Described below are non-limiting pharmaceutical compositions and methods for preparing the same.

### Pharmaceutical Compositions for Oral Administration

In preferred embodiments, the disclosure provides a pharmaceutical composition for oral administration containing: a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein, and a pharmaceutical excipient suitable for administration.

In preferred embodiments, the disclosure provides a solid pharmaceutical composition for oral administration containing: (i) an effective amount of: a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, and (ii) a pharmaceutical excipient suitable for administration. In some embodiments, the composition further contains (iii) an effective amount of an additional active pharmaceutical ingredient. For example, additional active pharmaceutical ingredients, as used herein, may include one or more compounds that are useful in the treatment or prevention of a disease or condition, such as a disease or condition selected from a depressive disorder, an anxiety disorder, drug addiction, schizophrenia, Alzheimer's dementia, amyotrophic lateral sclerosis, complex regional pain syndrome (CRPS), chronic pain, neuropathic pain, anhedonia, and fatigue.

Non-limiting examples of additional active pharmaceutical ingredients or agents include:
Antidepressants: ketamine, (R)-ketamine, (S)-ketamine, (2R,6R)-hydroxynorketamine (HNK), escitalopram, fluoxetine, paroxetine, duloxetine, sertraline, citalopram, bupropion, venlafaxine, duloxetine, naltrexone, mirtazapine, venlafaxine, atomoxetine, bupropion, doxepin, amitriptyline, clomipramine, nortriptyline, buspirone, aripiprazole, clozapine, loxapine, olanzapine, quetiapine, risperidone, ziprasidone, carbamazepine, gabapentin, lamotrigine, phenytoin, pregabalin, donepezil, galantamine, memantine, rivastigmine, tramiprosate, or pharmaceutically active salts or prodrugs thereof, or a combination of the foregoing;
Schizophrenia Medications: aripiprazole, lurasidone, asenapine, clozapine, ziprasidone, risperidone, quetiapine, stelazine, olanzapine, loxapine, flupentioxol, perphenazine, haloperidol, chlorpromazine, fluphenazine, prolixin, paliperidone;
Alzheimer's Dementia Medications: donepezil, rivastigmine, galantamine, memantine;
ALS Medications: riluzole;
Pain Medications: acetaminophen, aspirin, NSAIDS, including Diclofenac, Diflunisal, Etodolac, Fenoprofen, Flurbiprofen, Ibuprofen, Indomethacin, Ketoprofen, Ketorolac, Meclofenamate, Mefenamic Acid, Meloxicam, Nabumetone, Naproxen, Oxaprozin, Phenylbutazone, Piroxicam, Sulindac, Tolmetinopiods, Cox-2 inhibitors such as celcoxib, and narcotic pain medications such as Buprenorphine, Butorphanol, Codeine, Hydrocodone, Hydromorphone, Levorphanol, Meperidine, Methadone, Morphine, Nalbuphine, Oxycodone, Oxymorphone, Pentazocine, Propoxyphene, the central analgesic tramadol;
Medications for treatment of drug addiction: benzodiazepines, antidepressants, buprenorphine, methadone, naltrexone, clonidine, and naloxone;
CNS active agents: d-cycloserine, dextromethorphan, escitalopram, fluoxetine, paroxetine, duloxetine, sertraline, citalopram, bupropion, venlafaxine, duloxetine, naltrexone, mirtazapine, venlafaxine, atomoxetine, bupropion, doxepin, amitriptyline, clomipramine, nortriptyline, vortioxetine, vilazadone, milnacipran, levomilacipran, pramipexole, buspirone, lithium, thyroid or other type of hormones (e.g., estrogen, progesterone, testosterone), aripiprazole, brexpiprazole, cariprazine, clozapine, loxapine, lurasidone, olanzapine, paliperidone, quetiapine, risperidone, ziprasidone, carbamazepine, oxcarbazepine, gabapentin, lamotrigine, phenytoin, pregabalin, donepezil, galantamine, memantine, minocycline, rivastigmine, riluzole, tramiprosate, ketamine, or pharmaceutically active salts or prodrugs thereof, or a combination of the foregoing;
Anti-anxiety and anti-psychotic drugs: hydroxyzine hydrochloride, lorazepam, buspirone hydrochloride, pazepam, chlordiazepoxide, meprobamate, oxazepam, trifluoperazine, clorazepate dipotassium, diazepam, clozapine, prochlorperazine, haloperidol, thioridazine, thiothixene, risperidone, trifluoperazine hydrochloride, chlorpromazine, and related substances.

In some embodiments, the pharmaceutical composition may be a liquid pharmaceutical composition suitable for oral consumption.

Pharmaceutical compositions of the disclosure suitable for oral administration can be presented as discrete dosage forms, such as capsules, sachets, or tablets, or liquids or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The disclosure further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the disclosure can be prepared using anhydrous or low moisture containing ingredients and low moisture or low humidity conditions. Pharmaceutical compositions and dosage forms of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Active pharmaceutical ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmaceutical ingredient(s) may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acyllactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof, polyoxyethylated vitamins and derivatives thereof, polyoxyethylene-polyoxypropylene block copolymers; and mixtures thereof, polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present disclosure and to minimize precipitation of the compound of the present disclosure. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In preferred embodiments, the disclosure provides a pharmaceutical composition for injection containing: a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein, and a pharmaceutical excipient suitable for injection. Components and amounts of compounds in the compositions are as described herein.

The forms in which the compositions of the disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, and thimerosal.

Sterile injectable solutions are prepared by incorporating a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein, in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical Delivery

In preferred embodiments, the disclosure provides a pharmaceutical composition for transdermal delivery containing: a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein, and a pharmaceutical excipient suitable for transdermal delivery.

Compositions of the present disclosure can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present disclosure employs transdermal delivery devices (“patches”). Such transdermal patches may be used to provide continuous or discontinuous infusion of: a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein, in controlled amounts, either with or without another active pharmaceutical ingredient.

The construction and use of transdermal patches for the delivery of pharmaceutical agents is well known in the art. See, e.g., U.S. Pat. Nos. 5,023,252; 4,992,445 and 5,001,139. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner. Dry powder inhalers may also be used to provide inhaled delivery of the compositions.

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, et al., eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

Administration of a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein, or a pharmaceutical composition of these compounds can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. The compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein, can also be administered intraadiposally or intrathecally.

The compositions of the disclosure may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the disclosure may slow or inhibit the migration and proliferation of smooth muscle cells in the arterial wall which contribute to restenosis. A compound of the disclosure may be administered, for example, by local delivery from the struts of a stent, from a stent graft, from grafts, or from the cover or sheath of a stent. In some embodiments, a compound of the disclosure is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly(ether-ester) copolymers (e.g., PEO-PLLA); polydimethylsiloxane, poly(ethylene-vinylacetate), acrylate-based polymers or copolymers (e.g., polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. A compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein, may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the disclosure in a suitable solvent, followed by evaporation of the solvent. Excess drug on the surface of the stent may be removed via an additional brief solvent wash. In yet other embodiments, compounds of the disclosure may be covalently linked to a stent or graft. A covalent linker may be used which degrades in vivo, leading to the release of the compound of the disclosure. Any bio-labile linkage may be used for such a purpose, such as ester, amide or anhydride linkages. A compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein, may additionally be administered intravascularly from a balloon used during angioplasty. Extravascular administration of a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein, via the pericard or via advential application of formulations of the disclosure may also be performed to decrease restenosis.

Exemplary parenteral administration forms include solutions or suspensions of a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, in sterile aqueous solutions, for example, aqueous propylene glycol or dextrose solutions. Such dosage forms can be suitably buffered, if desired.

The disclosure also provides kits. The kits include a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein, in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In some embodiments, the compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein, and another active pharmaceutical ingredient are provided as separate compositions in separate containers within the kit. In some embodiments, the compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

The kits described above are preferably for use in the treatment of the diseases and conditions described herein. In some embodiments, the kits described herein are for use in the treatment of a disease or condition selected from a depressive disorder, an anxiety disorder, drug addiction, schizophrenia, Alzheimer's dementia, amyotrophic lateral sclerosis, complex regional pain syndrome (CRPS), chronic pain, and neuropathic pain.

### Dosages and Dosing Regimens

The amounts of: a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein, administered will be dependent on the human or mammal being treated, the severity of the disorder or condition, the rate of administration, the disposition of the compounds and the discretion of the prescribing physician. However, an effective dosage of each is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein, may be provided in units of mg/kg of body mass or in mg/m² of body surface area.

In some embodiments, a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein is administered in multiple doses. In a preferred embodiment, a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein is administered in multiple doses. Dosing may be once, twice, three times, four times, five times, six times, or more than six times per day. Dosing may be once a month, once every two weeks, once a week, or once every other day. In other embodiments, a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein, is administered about once per day to about 6 times per day. In some embodiments, a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein, is administered once daily, while in other embodiments, a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein is administered twice daily, and in other embodiments a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein, is administered three times daily.

Administration a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein, may continue as long as necessary. In some embodiments, a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein, is administered for more than 1, 2, 3, 4, 5, 6, 7, 14, or 28 days. In some embodiments, a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein is administered for less than 28, 14, 7, 6, 5, 4, 3, 2, or 1 day. In some embodiments, a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein is administered chronically on an ongoing basis—e.g., for the treatment of chronic effects. In another embodiment, the administration of a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein, continues for less than about 7 days. In yet another embodiment, the administration continues for more than about 6, 10, 14, 28 days, two months, six months, or one year. In some cases, continuous dosing is achieved and maintained as long as necessary.

In some embodiments, an effective dosage of a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein, is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg.

In some embodiments, an effective dosage of a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein, is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some instances, dosage levels below the lower limit of the aforesaid ranges may be more than adequate, while in other cases still larger doses may be employed without causing any harmful side effect—e.g., by dividing such larger doses into several small doses for administration throughout the day.

An effective amount of a compound of formula (I), formula (II), formula (III), formula (Ia), formula (Ib), formula (Ic), formula (Id), formula (Ie), formula (If), formula (Ig), or formula (Ih), formula (IIa), formula (IIb), formula (IIc), formula (IId), formula (IIe), formula (IIf), formula (IIg), or formula (IIh), formula (IIIa), formula (IIIb), formula (IIIc), formula (IIId), formula (IIIe), formula (IIIf), formula (IIIg), formula (IIIh), formulas 1001-1168, or formulas 2001-2004, or pharmaceutically acceptable salt thereof, described herein, may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

### Examples

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Synthesis of (5R)-methyl-(2R,6R)-hydroxynorketamine

An example of a synthetic scheme for the preparation of (5R)-methyl-(2R,6R)-hydroxynorketamine ((5R)-methyl-(2R,6R)-HNK) is outlined in Scheme 1 below. (R)-dehydroxynorketamine 1 is treated with dimethyl lithium cuprate in diethyl ether to produce methylated compound 2, which is then acetylated via treatment with acetyl chloride and sodium bicarbonate in benzene to produce compound 3. Compound 3 is then reacted first with lithium diisopropylamine (LDA) and trimethylsilyl chloride (TMSCl), followed by treatment with m-meta-chloroperoxybenzoic acid (mCPBA) and then tetrabutylammonium fluroride (TBAF) to provide compound 4. Finally, the amine is deprotected by treatment with N, O-bistrifluoroacetamide (BSTFA) in pyridine and dichloromethane (DCM) followed by treatment with trimethylsilyl iodide (TMSI) in DCM to provide (5R)-methyl-(2R,6R)-hydroxynorketamine 5:

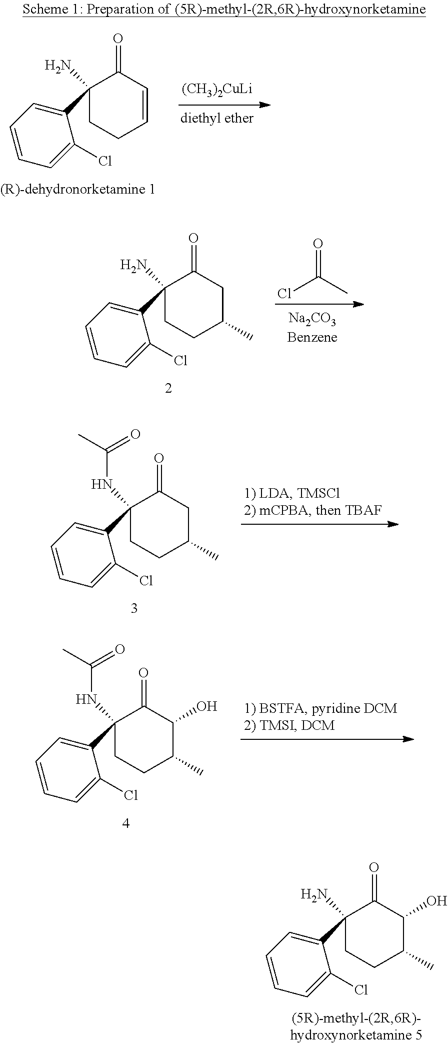

¹H NMR (400 MHz, Chloroform-d) δ 7.63 (dd, J=8.3, 1.6 Hz, 1H), 7.42-7.33 (m, 2H), 7.33-7.25 (m, 1H), 4.35 (d, J=6.7 Hz, 1H), 2.71 (dq, J=14.6, 3.0 Hz, 1H), 2.52 (ddqt, J=9.1, 6.9, 4.6, 2.1 Hz, 1H), 1.99-1.75 (m, 2H), 1.55 (dq, J=13.5, 2.9 Hz, 1H), 0.83 (d, J=7.1 Hz, 3H). Low resolution MS: expected: 253.7 (C₁₃H₁₆ClNO₂). Actual: 254.0

Absolute and relative stereochemistry were confirmed by single crystal X-ray crystallography.

In another example of a synthetic scheme for the preparation of the 5-methyl derivative of (2R,6R)-hydroxynorketamine, (R)-dehydronorketamine (1) was utilized as a starting material (Scheme 2). R)-Dehydronorketamine (1) was synthesized as previously reported by Morris et al., Org. Lett. 19:4572-4575 (2017), which is incorporated by reference herein in its entirety. A Gilman addition of dimethyl lithium cuprate to the enone gave the methylated cyclohexanone as a single diastereomer. The amine was immediately protected with a tert-butylcarbonate group and purified to give 2a. Silyl enol ether formation and a Rubottom oxidation resulted in the α-hydroxyketone 3a as a single diastereomer. Finally, a trifluoroacetic acid mediated deprotection of the amine, followed by formation of the free base, then formation of the HCl salt gave the desired product 5 as a single diastereomer. To conclusively establish the relative and absolute stereochemistry, single crystal X-ray crystallography was performed on the free base of 5 and conclusively established the 2R,4R,5R stereochemistry.

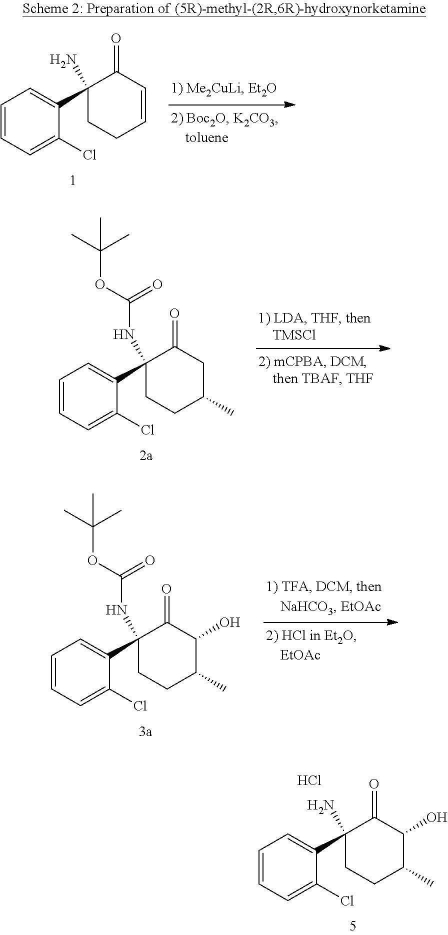

General Synthetic Methods: All commercially available reagents and solvents were purchased and used without further purification. All microwave reactions were carried out in a sealed microwave vial equipped with a magnetic stir bar and heated in a Biotage Initiator Microwave Synthesizer. ¹H NMR and ¹³C NMR spectra were recorded on Varian 400 MHz spectrometer in CD₃OD, or CDCl₃, as indicated. For spectra recorded in CD3OD, chemical shifts are reported in ppm with CD₃OD (3.31 ppm) as reference for ¹H NMR spectra and CD₃OD (49.0 ppm) for ¹³C NMR spectra. For spectra recorded in CDCl₃, chemical shifts are reported in ppm relative to deuterochloroform (7.26 ppm for ¹H NMR, 77.23 ppm for ¹³C NMR). The coupling constants (Jvalue) are reported as Hertz (Hz). The splitting patterns of the peaks were described as: singlet (s); doublet (d); triplet (t); quartet (q); multiplet (m) and septet (septet). Samples were analyzed for purity on an Agilent 1200 series LC/MS equipped with a Luna C18 (3 mm×75 mm, 3 μm) reversed-phase column with UV detection at λ=220 nm and λ=254 nm. The mobile phase consisted of water containing 0.05% trifluoroacetic acid as component A and acetonitrile containing 0.025% trifluoroacetic acid as component B. A linear gradient was run as follows: 0 min 4% B; 7 min 100% B; 8 min 100% B at a flow rate of 0.8 ml/min. High resolution mass spectrometry (HRMS) was recorded on Agilent 6210 Time-of-Flight (TOF) LC/MS system. Optical rotations were measured on an Anton Paar polarimeter using a 10 cm cell, at 589 nM and room temperature (20° C.). Optical rotations were measured on the free base, unless otherwise noted.

Compound Synthesis and Characterization

### tert-Butyl ((JR,4R)-1-(2-chlorophenyl)-4-methyl-2-oxocyclohexyl)carbamate (2a)

NCGC00689575

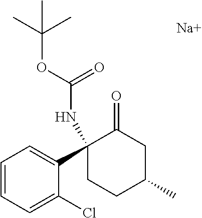 [SMILES: C[C@@H]1CC[C@@](NC(=O)OC(C)(C)C)(c2ccccc2Cl)C(=O)C1 ; [Na+]]

Copper (I) iodide (640 mg, 3.36 mmol) was added to a round bottom flask containing a stirbar. The flask was sealed with a rubber septa, placed under vacuum, and backfilled with nitrogen via a nitrogen balloon. To this flask was added diethyl ether (10.0 mL). The flask was cooled to −78° C. via a dry ice-acetone bath. Then methyl lithium (4.5 mL, 1.6 M in diethyl ether, 7.2 mmol) was added via syringe. The resulting mixture was stirred for 0.5 hours at −78° C. then removed from the dry ice-acetone bath and stirred a further 0.25 hours at room temperature, forming a pale light yellow solution. The dimethyl-lithium-cuprate solution was then cooled to −78° C.

(R)-dehydronorketamine (1) (532 mg, 2.40 mmol) was added to a separate flask containing a stirbar. The flask was sealed with a rubber septa, placed under vacuum, and backfilled with nitrogen via a nitrogen balloon. Then diethyl ether (10.0 mL) was added via syringe, and the flask was cooled to −78° C. via a dry ice-acetone bath. The dimethyl-lithium-cuprate solution in the first flask was then added to the dehydronorketamine solution via syringe, resulting in a cloudy bright orange suspension. The reaction was stirred for 2.5 hours at −78° C., then removed from the dry-ice acetone bath and allowed to stir at room temperature for an additional 0.5 hours. The reaction was quenched by being poured into a solution of saturated aqueous sodium bicarbonate, and was extracted with ethyl acetate. The ethyl acetate extraction (including the emulsion layer) was then washed with a saturated solution of ammonium chloride, which cleared the emulsion. The ethyl acetate organic phase was taken. The aqueous ammonium chloride layer was made basic by the addition of concentrated ammonium hydroxide, and extracted with a second aliquot of ethyl acetate. The two ethyl acetate layers were combined, and the solvent was removed in vacuo to give the crude methylated intermediate.

The crude methylated intermediate was placed in a round bottom flask containing a stirbar. Then Boc-anhydride (800 mg, 3.6 mmol) was added to the flask, followed by toluene (20.0 mL) and potassium carbonate (276 mg, 2.0 mmol). An air-cooled reflux condenser was added and the reaction was placed under nitrogen and heated to 85° C. for 18 hours. The reaction was cooled, poured into 50% saturated aqueous sodium bicarbonate and extracted with ethyl acetate. The organic phase was taken, and the solvent removed in vacuo. Purification by silica gel chromatography (0% to 100% ethyl acetate in hexanes) gave the desired product in 65.2% yield (528 mg) as a single diastereomer. ¹H NMR (400 MHz, CDCl₃) δ 7.85-7.79 (m, 1H), 7.38-7.32 (m, 2H), 7.30-7.21 (m, 1H), 6.51 (s, 1H), 3.60 (d, J=14.3 Hz, 1H), 2.57 (dd, J=11.6, 5.9 Hz, 1H), 2.46-2.37 (m, 1H), 2.20 (dt, J=11.6, 2.6 Hz, 1H), 2.04 (tt, J=13.3, 4.1 Hz, 1H), 1.92 (td, J=13.8, 3.4 Hz, 1H), 1.57-1.49 (m, 1H), 1.32 (s, 9H), 1.00 (d, J=7.1 Hz, 3H).δ. HRMS (ESI+): Expected [M+Na⁺]360.1337 (C₁₈H₂₄ClN₂NaO₃+). Observed: 360.1323 [α]_{D}²⁰: −60.2° (c 1.0, CDCl₃)

### tert-Butyl ((JR,3R,4R)-1-(2-chlorophenyl)-3-hydroxy-4-methyl-2-oxocyclohexyl)carbamate (3)

NCGC00689494

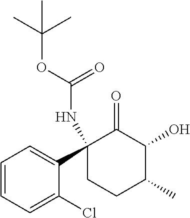 [SMILES: C[C@@H]1CC[C@@](NC(=O)OC(C)(C)C)(c2ccccc2Cl)C(=O)[C@@H]1O]

tert-Butyl ((JR,4R)-1-(2-chlorophenyl)-4-methyl-2-oxocyclohexyl)carbamate (2a) (330 mg, 0.977 mmol) was added to a round bottom flask containing a stirbar. The flask was sealed, placed under vacuum, then backfilled with nitrogen. Tetrahydrofuran (10.0 mL) was added. Then the flask was cooled to −50° C. with a dry ice cooled acetone bath (carefully monitored to keep the temperature between −50° C. and −40° C. by periodic addition of small quantities of dry ice). Lithium diisopropyl amide (1.47 mL, 2.0 M in THF/heptanes/ethylbenzene, 2.94 mmol) was added and the reaction was allowed to stir for 0.75 h at −45° C. (+/−5° C.). Then clorotrimethylsilane (318 mg, 0.375 mL, 2.93 mmol) was added via syringe. The reaction was stirred to 0.5 hours at −45° C., then a further 0.25 hours at room temperature. It was quenched by being poured into saturated aqueous ammonium chloride and extracted with ethyl acetate. The organic phase was taken and the solvent removed in vacuo. Purification by silica gel chromatography (0% to 50% ethyl acetate in hexanes) gave the purified silyl enol ether (250 mg) which was immediately used.

The purified enol ether (250 mg) was dissolved in dichloromethane (20 ml) and transferred to a round bottom flask with a stirbar. The flask was cooled to 0° C. and meta-chlorperoxybenzoic acid (150 mg, 77% by weight, 0.671 mmol) was added as a solid. The reaction was allowed to stir for 3 hours and slowly warm to room temperature. The reaction was then quenched by being poured into saturated aqueous sodium bicarbonate, extracted into dichloromethane, the organic layer taken, and the solvent removed in vacuo. The resulting crude material was dissolved in THE (15 mL) and tetrabutylammonium fluoride (0.610 mL, 1 M in THF, 0.610 mmol was added). The reaction was stirred for two minutes, then quenched by being poured into saturated aqueous sodium bicarbonate. The mixture was extracted with ethyl acetate, the organic phase taken, and the solvent removed in vacuo. Purification by silica gel chromatography (0% to 100% ethyl acetate in hexanes) gave the desired product in 39% overall yield (143 mg). ¹H NMR (400 MHz, CDCl₃) δ. 7.87-7.81 (m, 1H), 7.41-7.32 (m, 2H), 7.32-7.26 (m, 1H), 6.60 (s, 1H), 4.29 (t, J=6.3 Hz, 1H), 3.73 (d, J=14.8 Hz, 1H), 3.28 (d, J=6.7 Hz, 1H), 2.56 (dtd, J=9.1, 5.6, 4.3, 2.1 Hz, 1H), 2.07-1.95 (m, 1H), 1.86 (t, J=14.8 Hz, 1H), 1.32 (s, 9H), 0.83 (d, J=7.1 Hz, 3H). HRMS (ESI+): Expected [M+Na⁺]376.1286 (C₁₈H₂₄ClN₂NaO₄*). Observed. 376.1297 [α]_{D}²⁰: −69.7° (c 1.0, CDCl₃)

### (2R,5R,6R)-2-amino-2-(2-chlorophenyl)-6-hydroxy-5-methylcyclohexan-1-one hydrochloride (5) (5R-Methyl-2R,6R-hydronorketamine)

NCGC00522050

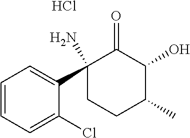 [SMILES: Cl ; C[C@@H]1CC[C@@](N)(c2ccccc2Cl)C(=O)[C@@H]1O]

tert-Butyl ((JR,3R,4R)-1-(2-chlorophenyl)-3-hydroxy-4-methyl-2-oxocyclohexyl)carbamate (3a) (101 mg, 0.285 mmol) was added to a vial containing a stirbar. Dichloromethane (2 mL) was added to the vial, followed by trifluoroacetic acid (1.5 mL, 2.2 grams, 19.5 mmol). The reaction was stirred for 0.5 hours at room temperature. The reaction was then halted, the stir bar removed, and both the solvent and trifluoroacetic acid were removed by rotary evaporation to give the desired product, as a triflate salt. This product was dissolved in water, and added to a solution of saturated aqueous sodium bicarbonate This solution was extracted with ethyl acetate (2X), the organic phases taken and combines, and the solvent removed in vacuo to give the free base. This free base was dissolved in ethyl acetate (4 mL), and an ethereal solution of hydrogen chloride was added via syringe (1.0 ml, 2.0 M in diethyl ether, 1.0 mmol) to give a white solid suspension. The organic solvent was removed by rotary evaporation, and the material dried under vacuum to give the final product as a white solid in 89% yield (74 mg). ¹H NMR (400 MHz, Methanol-d₄) δ 7.91 (d, J=7.2 Hz, 1H), 7.64-7.53 (m, 3H), 4.47 (dd, J=6.6, 1.0 Hz, 1H), 3.03 (d, J=14.4 Hz, 1H), 2.58-2.50 (m, 1H), 2.18 (td, J=14.2, 3.6 Hz, 1H), 2.06-1.93 (m, 1H), 1.71 (dd, J=14.6, 2.9 Hz, 1H), 0.93 (d, J=7.1 Hz, 3H). ¹³C NMR (101 MHz, Methanol-d₄) δ 206.57, 135.57, 133.57, 133.15, 132.06, 131.29, 129.73, 76.42, 68.15, 41.57, 32.74, 26.60, 11.66. HRMS (ESI+): Expected [M+H⁺]: 254.0948 (C₁₃H₁₆ClNO₂⁺). Observed. 254.0951 [α]_{D}²⁰: −106.8 degrees (c 1.0, EtOH)

Experimental Summary

The absolute and relative stereochemistry for (5R)-methyl-(2R,6R)-hydroxynorketamine (NIH35) was established unambiguously by single crystal x-ray crystallography. The single crystal X-ray diffraction studies were carried out on a Bruker Kappa APEX-II CCD diffractometer equipped with Mo Kₐ radiation (1=0.71073 Å). A 0.153×0.147×0.144 mm piece of a colorless block was mounted on a Cryoloop with Paratone oil. Data were collected in a nitrogen gas stream at 100(2) K using f and v scans. Crystal-to-detector distance was 40 mm and exposure time was 5 seconds per frame using a scan width of 2.0°. Data collection was 100% complete to 25.00° in q. A total of 19959 reflections were collected covering the indices, −8<=h<=8, −13<=k<=13, −10<=1<=10. 2512 reflections were found to be symmetry independent, with a Rᵢₙₜ of 0.0540. Indexing and unit cell refinement indicated a primitive, monoclinic lattice. The space group was found to be P2₁. The data were integrated using the Bruker SAINT software program and scaled using the SADABS software program. Solution by direct methods (SHELXT) produced a complete phasing model consistent with the proposed structure.

All nonhydrogen atoms were refined anisotropically by full-matrix least-squares (SHELXL-2014). All carbon bonded hydrogen atoms were placed using a riding model. Their positions were constrained relative to their parent atom using the appropriate HFIX command in SHELXL-2014. All other hydrogen atoms (H-bonding) were located in the difference map. Their relative positions were restrained using DFIX commands and their thermals freely refined. The absolute stereochemistry of the molecule was established by anomalous dispersion using the Parson's method with a Flack parameter of −0.026(27). FIG. 3 illustrates the 3-dimensional structure of (5R)-methyl-(2R,6R)-hydroxynorketamine. Crystallographic data are summarized in Tables 1-6.

TABLE 1
Crystal data and structure refinement for
(5R)-methyl-(2R,6R)-hydroxynorketamine.

| Identification code | PJM-013-014 | |
|---|---|---|
| Empirical formula | C13 H16 Cl N O2 | |
| Molecular formula | C13 H16 Cl N O2 | |
| Formula weight | 253.72 | |
| Temperature | 100.0 K | |
| Wavelength | 0.71073 Å | |
| Crystal system | Monoclinic | |
| Space group | P 1 21 1 | |
| Unit cell dimensions | a = 6.5842(9) Å | α = 90°. |
| | b = 11.0230(14) Å | β = 101.266(4)°. |
| | c = 8.6030(12) Å | γ = 90°. |

| | |
|---|---|
| Volume | 612.35(14) Å³ |
| Z | 2 |
| Density (calculated) | 1.376 Mg/m³ |
| Absorption coefficient | 0.301 mm⁻¹ |
| F(000) | 268 |
| Crystal size | 0.153 × 0.147 × 0.144 mm³ |
| Crystal color, habit | Colorless Block |
| Theta range for data collection | 2.414 to 26.401°. |
| Index ranges | −8 <= h <= 8, −13 <= k <= 13, −10 <= l <= 10 |
| Reflections collected | 19959 |
| Independent reflections | 2512 [R(int) = 0.0540, R(sigma) = 0.0321] |
| Completeness to theta = 25.000° | 100.0% |
| Absorption correction | Semi-empirical from equivalents |
| Max. and min. transmission | 0.2580 and 0.2321 |
| Refinement method | Full-matrix least-squares on F² |
| Data/restraints/parameters | 2512/4/167 |
| Goodness-of-fit on F² | 1.033 |
| Final R indices [I > 2sigma(I)] | R1 = 0.0270, wR2 = 0.0617 |
| R indices (all data) | R1 = 0.0298, wR2 = 0.0638 |
| Absolute structure parameter | −0.03(3) |
| Extinction coefficient | n/a |
| Largest diff. peak and hole | 0.213 and −0.157 e · Å⁻³ |

TABLE 2
Atomic coordinates (×10⁴) and equivalent isotropic
displacement parameters (Å² × 10³) for
(5R)-methyl-(2R,6R)-hydroxynorketamine.

| | x | y | z | U(eq) |
|---|---|---|---|---|
| Cl(1) | 8569(1) | 6763(1) | 2274(1) | 26(1) |
| O(1) | 10544(2) | 5396(2) | 5531(2) | 22(1) |
| O(2) | 7884(3) | 6666(2) | 7019(2) | 23(1) |
| N(1) | 9609(3) | 3769(2) | 3151(2) | 18(1) |
| C(1) | 6375(4) | 5861(2) | 1714(3) | 16(1) |
| C(2) | 4878(4) | 6284(2) | 455(3) | 20(1) |
| C(3) | 3140(4) | 5586(2) | −99(3) | 21(1) |
| C(4) | 2933(4) | 4475(2) | 606(3) | 19(1) |
| C(5) | 4418(3) | 4076(2) | 1876(3) | 16(1) |
| C(6) | 6181(3) | 4760(2) | 2481(3) | 14(1) |
| C(7) | 7870(3) | 4255(2) | 3815(3) | 14(1) |
| C(8) | 8712(4) | 5199(2) | 5109(3) | 15(1) |
| C(9) | 7091(4) | 5743(2) | 5937(3) | 16(1) |
| C(10) | 6184(3) | 4723(2) | 6830(3) | 16(1) |
| C(11) | 5412(3) | 3688(2) | 5682(3) | 17(1) |
| C(12) | 7047(3) | 3236(2) | 4771(3) | 16(1) |
| C(13) | 7769(4) | 4296(2) | 8269(3) | 20(1) |

U(eq) is defined as one third of the trace of the orthogonalized Uij tensor.

TABLE 3
Bond lengths [Å] and angles [°] for (5R)-methyl-(2R,6R)-hydroxynorketamine

| | | | |
|---|---|---|---|
| Cl(1)—C(1) | 1.742(2) | C(12)—H(12A) | 0.9900 |
| O(1)—C(8) | 1.209(3) | C(12)—H(12B) | 0.9900 |
| O(2)—H(2) | 0.90(2) | C(13)—H(13A) | 0.9800 |
| O(2)—C(9) | 1.409(3) | C(13)—H(13B) | 0.9800 |
| N(1)—H(1A) | 0.91(2) | C(13)—H(13C) | 0.9800 |
| N(1)—H(1B) | 0.93(2) | | |
| N(1)—C(7) | 1.477(3) | C(9)—O(2)—H(2) | 112(2) |
| C(1)—C(2) | 1.395(3) | H(1A)—N(1)—H(1B) | 102(3) |
| C(1)—C(6) | 1.399(3) | C(7)—N(1)—H(1A) | 107(2) |
| C(2)—H(2A) | 0.9500 | C(7)—N(1)—H(1B) | 108(2) |
| C(2)—C(3) | 1.384(3) | C(2)—C(1)—Cl(1) | 116.48(18) |
| C(3)—H(3) | 0.9500 | C(2)—C(1)—C(6) | 122.7(2) |
| C(3)—C(4) | 1.385(4) | C(6)—C(1)—Cl(1) | 120.79(17) |
| C(4)—H(4) | 0.9500 | C(1)—C(2)—H(2A) | 120.2 |
| C(4)—C(5) | 1.388(3) | C(3)—C(2)—C(1) | 119.6(2) |
| C(5)—H(5) | 0.9500 | C(3)—C(2)—H(2A) | 120.2 |
| C(5)—C(6) | 1.397(3) | C(2)—C(3)—H(3) | 120.5 |
| C(6)—C(7) | 1.538(3) | C(2)—C(3)—C(4) | 119.1(2) |
| C(7)—C(8) | 1.545(3) | C(4)—C(3)—H(3) | 120.5 |
| C(7)—C(12) | 1.551(3) | C(3)—C(4)—H(4) | 119.6 |
| C(8)—C(9) | 1.517(3) | C(3)—C(4)—C(5) | 120.7(2) |
| C(9)—H(9) | 1.0000 | C(5)—C(4)—H(4) | 119.6 |
| C(9)—C(10) | 1.545(3) | C(4)—C(5)—H(5) | 119.1 |
| C(10)—H(10) | 1.0000 | C(4)—C(5)—C(6) | 121.9(2) |
| C(10)—C(11) | 1.530(3) | C(6)—C(5)—H(5) | 119.1 |
| C(10)—C(13) | 1.529(3) | C(1)—C(6)—C(7) | 123.2(2) |
| C(11)—H(11A) | 0.9900 | C(5)—C(6)—C(1) | 116.0(2) |
| C(11)—H(11B) | 0.9900 | C(5)—C(6)—C(7) | 120.6(2) |
| C(11)—C(12) | 1.534(3) | N(1)—C(7)—C(6) | 110.19(17) |
| N(1)—C(7)—C(8) | 109.09(18) | H(13A)—C(13)—H(13C) | 109.5 |
| N(1)—C(7)—C(12) | 108.65(19) | H(13B)—C(13)—H(13C) | 109.5 |
| C(6)—C(7)—C(8) | 113.55(19) | | |
| C(6)—C(7)—C(12) | 112.42(18) | | |
| C(8)—C(7)—C(12) | 102.61(18) | | |
| O(1)—C(8)—C(7) | 122.3(2) | | |
| O(1)—C(8)—C(9) | 122.7(2) | | |
| C(9)—C(8)—C(7) | 114.70(19) | | |
| O(2)—C(9)—C(8) | 113.07(19) | | |
| O(2)—C(9)—H(9) | 108.6 | | |
| O(2)—C(9)—C(10) | 109.01(18) | | |
| C(8)—C(9)—H(9) | 108.6 | | |
| C(8)—C(9)—C(10) | 108.75(19) | | |
| C(10)—C(9)—H(9) | 108.6 | | |
| C(9)—C(10)—H(10) | 107.9 | | |
| C(11)—C(10)—C(9) | 109.49(18) | | |
| C(11)—C(10)—H(10) | 107.9 | | |
| C(13)—C(10)—C(9) | 110.96(18) | | |
| C(13)—C(10)—H(10) | 107.9 | | |
| C(13)—C(10)—C(11) | 112.4(2) | | |
| C(10)—C(11)—H(11A) | 108.9 | | |
| C(10)—C(11)—H(11B) | 108.9 | | |
| C(10)—C(11)—C(12) | 113.19(18) | | |
| H(11A)—C(11)—H(11B) | 107.8 | | |
| C(12)—C(11)—H(11A) | 108.9 | | |
| C(12)—C(11)—H(11B) | 108.9 | | |
| C(7)—C(12)—H(12A) | 109.0 | | |
| C(7)—C(12)—H(12B) | 109.0 | | |
| C(11)—C(12)—C(7) | 112.90(19) | | |
| C(11)—C(12)—H(12A) | 109.0 | | |
| C(11)—C(12)—H(12B) | 109.0 | | |
| H(12A)—C(12)—H(12B) | 107.8 | | |
| C(10)—C(13)—H(13A) | 109.5 | | |
| C(10)—C(13)—H(13B) | 109.5 | | |
| C(10)—C(13)—H(13C) | 109.5 | | |
| H(13A)—C(13)—H(13B) | 109.5 | | |

TABLE 4
Anisotropic displacement parameters (Å² × 10³) for
(5R)-methyl-(2R,6R)-hydroxynorketamine

| | U¹¹ | U²² | U³³ | U²³ | U¹³ | U¹² |
|---|---|---|---|---|---|---|
| Cl(1) | 26(1) | 21(1) | 28(1) | 6(1) | −1(1) | −10(1) |
| O(1) | 16(1) | 24(1) | 24(1) | −2(1) | 0(1) | −4(1) |
| O(2) | 34(1) | 15(1) | 22(1) | −5(1) | 11(1) | −7(1) |
| N(1) | 15(1) | 20(1) | 21(1) | −3(1) | 5(1) | 3(1) |
| C(1) | 16(1) | 17(1) | 16(1) | −2(1) | 4(1) | −2(1) |
| C(2) | 26(1) | 17(1) | 18(1) | 3(1) | 6(1) | 3(1) |
| C(3) | 18(1) | 31(2) | 14(1) | 2(1) | 2(1) | 7(1) |
| C(4) | 15(1) | 26(1) | 16(1) | −5(1) | 2(1) | −2(1) |
| C(5) | 18(1) | 18(1) | 13(1) | −3(1) | 5(1) | −1(1) |
| C(6) | 15(1) | 16(1) | 13(1) | −2(1) | 4(1) | 2(1) |
| C(7) | 14(1) | 13(1) | 16(1) | −1(1) | 3(1) | 1(1) |
| C(8) | 17(1) | 12(1) | 15(1) | 3(1) | 2(1) | −2(1) |
| C(9) | 19(1) | 13(1) | 15(1) | −2(1) | 2(1) | 2(1) |
| C(10) | 14(1) | 19(1) | 15(1) | 0(1) | 4(1) | 0(1) |
| C(11) | 17(1) | 19(1) | 16(1) | 0(1) | 3(1) | −4(1) |
| C(12) | 17(1) | 14(1) | 16(1) | 2(1) | 1(1) | −1(1) |
| C(13) | 23(1) | 21(1) | 16(1) | 2(1) | 2(1) | −2(1) |

The anisotropic displacement factor exponent takes the form: −2π²[h² a*²U¹¹ + . . . + 2 h k a* b* U¹²]

TABLE 5
Hydrogen coordinates (×10⁴) and isotropic
displacement parameters (Å² × 10³)
For (5R)-methyl-(2R,6R)-hydroxynorketamine

| | x | y | z | U(eq) |
|---|---|---|---|---|
| H(2) | 8860(50) | 7110(30) | 6690(40) | 50(11) |
| H(1A) | 10660(50) | 4310(30) | 3400(40) | 53(11) |
| H(1B) | 9280(60) | 3850(40) | 2060(30) | 73(13) |
| H(2A) | 5051 | 7045 | −20 | 24 |
| H(3) | 2103 | 5865 | −951 | 26 |
| H(4) | 1763 | 3981 | 215 | 23 |
| H(5) | 4229 | 3316 | 2349 | 19 |
| H(9) | 5952 | 6090 | 5117 | 19 |
| H(10) | 4967 | 5062 | 7220 | 19 |
| H(11A) | 4180 | 3968 | 4913 | 21 |
| H(11B) | 4976 | 3003 | 6286 | 21 |
| H(12A) | 6438 | 2585 | 4034 | 19 |
| H(12B) | 8223 | 2884 | 5531 | 19 |
| H(13A) | 8987 | 3968 | 7917 | 30 |
| H(13B) | 7150 | 3663 | 8828 | 30 |
| H(13C) | 8185 | 4982 | 8984 | 30 |

TABLE 6
Hydrogen bonds for (5R)-methyl-(2R,6R)-hydroxynorketamine
[Å and °].

| D-H . . . A | d(D—H) | d(H . . . A) | d(D . . . A) | <(DHA) |
|---|---|---|---|---|
| O(2)—H(2) . . . | 0.90(2) | 2.08(3) | 2.866(3) | 146(3) |
| N(1)#1 | | | | |

Symmetry transformations used to generate equivalent atoms: #1 −x + 2, y + 1/2, −z + 1

### Example 2: Evaluation of the Antidepressant-Relevant Behavioral Effectiveness of the (2,6)-hydroxynorketamines

The antidepressant-like behavioral effects of (2R,6R)-, (2S,6S)-, (2R,6S)-, and (2S,6R)-HNK were compared in the mouse forced swim test and their relative rank-order of effectiveness was established by comparing their minimally effective doses under the same experimental conditions. Additionally, the novel drug candidate (5R)-methyl-(2R,6R)-HNK was used, which shares a three-dimensional structure similar to (2R,6S)-HNK, to evaluate whether three-dimensional structure confers the behavioral effectiveness of the (2,6)-HNKs. As described in this example, (2R,6S)-HNK was identified to reduce forced swim test immobility (antidepressant assay) with greater effectiveness compared to the (2R,6R)-HNK molecule. PK studies have shown that (2R,6R)-HNK has higher exposure levels, so PK difference do not explain these results. It was hypothesized that differences in the three-dimensional structures of these compounds underlies their differential behavioral effectiveness. Specifically, while (2R,6R)-HNK prefers to localize the amine and hydroxyl groups equatorial and the aryl group axial to the cyclohexyl ring, (2R,6S)-HNK prefers to localize the aryl group equatorial. The novel compound (5R)-methyl-(2R,6R)-HNK was synthesized, and it was found that methyl group substitution causes the compound to adopt a three-dimensional structure akin to (2R,6S)-HNK. It was also found that the (5R)-methyl-(2R,6R)-HNK was more potent than the (2R,6R)-HNK, and of similar potency as the (2R,6S)-HNK.

Methods

Animals: Male CD-1 mice (Charles Rivers Laboratories; Raleigh, NC, USA), 8-11 weeks old at the time of testing, were habituated to the University of Maryland (Baltimore, MD, USA) animal facility for at least one week prior to testing. Mice were group housed in cages of 4-5 per cage with a constant 12-hour light cycle (lights on/off at 07:00/19:00). Food and water were available ad libitum. All experiments were performed during the light phase. All studies were approved by the University of Maryland School of Medicine Animal Care and Use Committee and conducted in accordance with the National Institutes of Health Guide for the Care and Use of Laboratory Animals.

Drugs: (2R,6R)-HNK hydrochloride, (2S,6S)-HNK hydrochloride, (2R,6S)-HNK, and (2S,6R)-HNK were synthesized and characterized (molecular structures were confirmed via ¹H and ¹³C NMR, the absolute and relative stereochemistry was assigned by x-ray crystallography, and the purity was determined via LC/MS) at the National Center for Advancing Translation Sciences (National Institutes of Health, Rockville, MD, USA). The synthesis and characterization of (2R,6R)-HNK hydrochloride, (2S,6S)-HNK hydrochloride, (2R,6S)-HNK, and (2S,6R)-HNK were performed as previously described in Morris et al., 2017, Org. Lett. 19:4572-4575, which is incorporated by reference herein in its entirety. (5R)-methyl-(2R,6R)-HNK was obtained from Organix Inc. (Woburn, MA, USA). (5R)-methyl-(2R,6R)-HNK can be synthesized according to the method described in Example 1. All compounds were dissolved in 20% (w/v) cyclodextrin (Sigma Aldrich, USA) in saline and administered intraperitoneally (i.p.) in a volume of 7.5 ml/kg.

Forced swim test: Compounds were tested in separate cohorts of male mice (n=10-18/group; only male mice were tested to control for sex-dependent differences in pharmacokinetics and brain levels) and all injections were performed by a male experimenter. (2R,6R)-HNK, (2S,6S)-HNK, (2R,6S)-HNK, (2S,6R)-HNK were administered at the doses of 1, 3, 10, 30, and 100 mg/kg. (5R)-methyl-(2R,6R)-HNK was administered at the doses of 0.3, 1, 3, and 10 mg/kg. (2R,6S)-HNK and (2S,6R)-HNK were dosed as a free base whereas (2R,6R)-HNK and (2S,6S)-HNK, and (5R)-methyl-(2R,6R)-HNK were dosed as a hydrochloride salt. Vehicle and (2R,6R)-HNK (30 mg/kg) were included as controls in each cohort. Twenty-four h after treatment, mice were tested in the forced swim test, according to previously described methods. Injections were performed by a male experimenter. Briefly, mice were placed into a clear Plexiglass cylinder (20 cm height×15 cm diameter) filled to a depth of 15 cm with water (23±1° C.) and subjected to a 6-min swim session. Swim sessions were recorded using a digital video camera. The time spent immobile (defined as passive floating with no movements other than those necessary to keep the head above water) was scored during the final 4 min of the session. Sessions were scored by a trained experimenter blind to the treatment groups.

Statistical analysis: All experiments and analyses were performed in a blinded and randomized manner. All injections were performed by a male experimenter. Statistical analyses were performed using GraphPad Prism software (v8; GraphPad Software, Inc.). All statistical tests were two-tailed, and significance was assigned at p<0.05. Differences in immobility time were analyzed via one-way ANOVA followed by Holm-Šídák post-hoc comparison (with all groups compared to saline control group) when significance was reached. Significant results are indicated with asterisks in the figures (*p<0.05, **p<0.01, ***p<0.001). Data are presented as the mean±standard error of the mean (SEM). Individual group sizes and statistical analyses for each experiment are summarized in Table 7.

TABLE 7
Statistical analyses of effects of the (2,6)-HNKs in the forced swim test

| | | | | | Holm-\check{S}ídák post- |
|---|---|---|---|---|---|
| | | | | | hoc |
| | | | | | comparison, |
| | | | Sample | Treatment effect, | group compared |
| HNK | Figure | Groups | size | one-way ANOVA | vehicle |
| (2R,6R)-HNK | 1A | vehicle | 16 | F_{[5, 84]} = 2.997 | NA |
| | | (2R,6R)-HNK, 1 mg/kg | 15 | p = 0.0154 | p = 0.2332 |

| | | | |
|---|---|---|---|
| 3 | mg/kg | 15 | p = 0.2631 |
| 10 | mg/kg | 15 | p = 0.0428 |
| 30 | mg/kg | 14 | p = 0.0135 |
| 100 | mg/kg | 15 | p = 0.0135 |

| | | | | | |
|---|---|---|---|---|---|
| (2S,6S)-HNK | 1B | vehicle | 13 | F_{[6, 82]} = 9.015 | NA |
| | | (2R,6R)-HNK, 30 mg/kg | 13 | p < 0.0001 | p = 0.0119 |
| | | (2S,6S)-HNK, 1 mg/kg | 12 | | p = 0.9176 |

| | | | |
|---|---|---|---|
| 3 | mg/kg | 12 | p = 0.8804 |
| 10 | mg/kg | 13 | p = 0.9176 |
| 30 | mg/kg | 13 | p = 0.0013 |
| 100 | mg/kg | 13 | p = 0.0003 |

| | | | | | |
|---|---|---|---|---|---|
| (2R,6S)-HNK | 1C | vehicle | 16 | F_{[6, 87]} = 2.940 | NA |
| | | (2R,6R)-HNK, 30 mg/kg | 18 | p = 0.0117 | p = 0.0090 |
| | | (2R,6S)-HNK, 1 mg/kg | 13 | | p = 0.0457 |

| | | | |
|---|---|---|---|
| 3 | mg/kg | 13 | p = 0.0261 |
| 10 | mg/kg | 13 | p = 0.0261 |
| 30 | mg/kg | 10 | p = 0.4950 |
| 100 | mg/kg | 11 | p = 0.4950 |

| | | | | | |
|---|---|---|---|---|---|
| (2S,6R)-HNK | 1D | vehicle | 15 | F_{[6, 91]} = 3.969 | NA |
| | | (2R,6R)-HNK, 30 mg/kg | 15 | p = 0.0014 | p = 0.0035 |
| | | (2S,6R)-HNK, 1 mg/kg | 13 | | p = 0.8466 |

| | | | |
|---|---|---|---|
| 3 | mg/kg | 14 | p = 0.0404 |
| 10 | mg/kg | 13 | p = 0.0404 |
| 30 | mg/kg | 14 | p = 0.8466 |
| 100 | mg/kg | 14 | p = 0.8466 |

| | | | | | |
|---|---|---|---|---|---|
| (5R)-Me-(2R,6R)- | 2F | vehicle | 15 | F_{[5, 79]} = 4.191 | NA |
| HNK | | (2R,6R)-HNK, 30 mg/kg | 14 | p = 0.0020 | p = 0.0396 |
| | | (5R)-Me-(2R,6R)-HNK, 0.3 mg/kg | 14 | | p = 0.8074 |

| | | | |
|---|---|---|---|
| 1 | mg/kg | 14 | p = 0.0257 |
| 3 | mg/kg | 15 | p = 0.0671 |
| 10 | mg/kg | 13 | p = 0.0026 |

Note:
samples sizes are listed in respective order of experimental groups. Abbreviations: HNK, hydroxynorketamine; Me, methyl; NA, not applicable; VEH, vehicle.

Results

(2,6)-HNKs Reduce Forced Swim Test Immobility Time with Differing Effectiveness

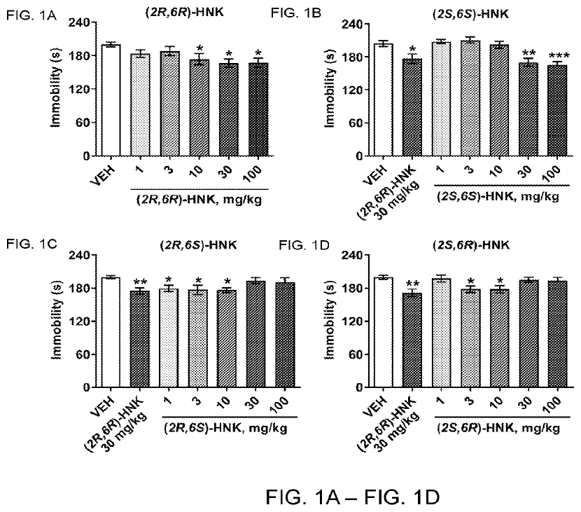

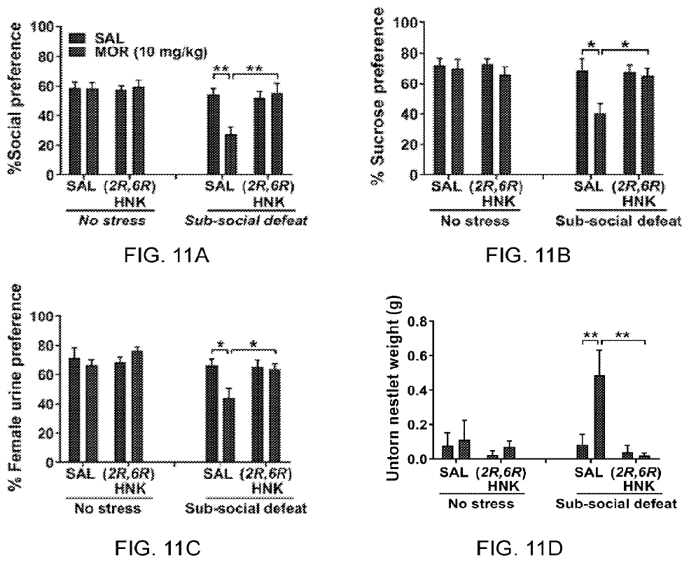

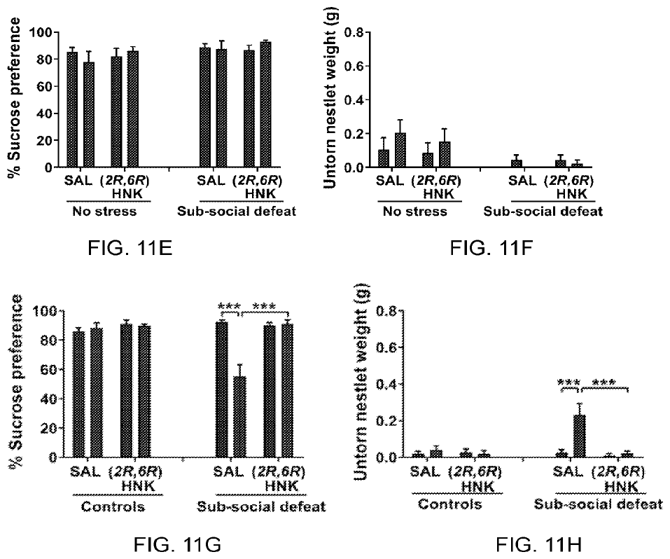

The behavioral effects of (2R,6R)-HINK (FIG. 1A), (2S,6S)-HINK (FIG. 11n), (2R,6S)-HNK((FIG. 1C), and (2S,6R)-HINK (FIG. 1D) were evaluated in the mouse forced swim test, 24 h after treatment (see Table 7 for statistical analyses). For mice treated with a particular compound, a reduction in immobility in the forced swim test is indicative of the anti-depressant action of the compound. Relative to vehicle, (2R,6R)-HINK reduced immobility time at 10, 30, and 100 mg/kg (FIG. 1A), whereas (2S,6S)-HINK reduced immobility at 30 and 100 mg/kg, consistent with previous reports of their relative behavioral effectiveness. (2R,6S)-HINK (FIG. 1C) and (2S,6R)-HNK (FIG. 1D) reduced immobility time at the doses of 1, 3, and 10 mg/kg and at 3 and 10 mg/kg, respectively. This demonstrates, for the first time, that (2R,6S)- and (2S,6R)-HNK are capable of exerting antidepressant-relevant behavioral effects, both with enhanced effectiveness compared to (2R,6R)-HNK. Of note, both (2R,6S)- and (2S,6R)-HNK exhibited U-shaped dose response curves, reducing immobility time at doses <10 mg/kg, but not at doses >30 mg/kg (FIGS. 1C-1D), in contrast with (2R,6R)- and (2S,6S)-HNK, which did not demonstrate U-shaped dose responses within the tested range (up to 100 mg/kg).

### (5R)-methyl-(2R,6R)-HNK has Comparable Antidepressant-Like Behavioral Effectiveness to (2R,6S)-HNK

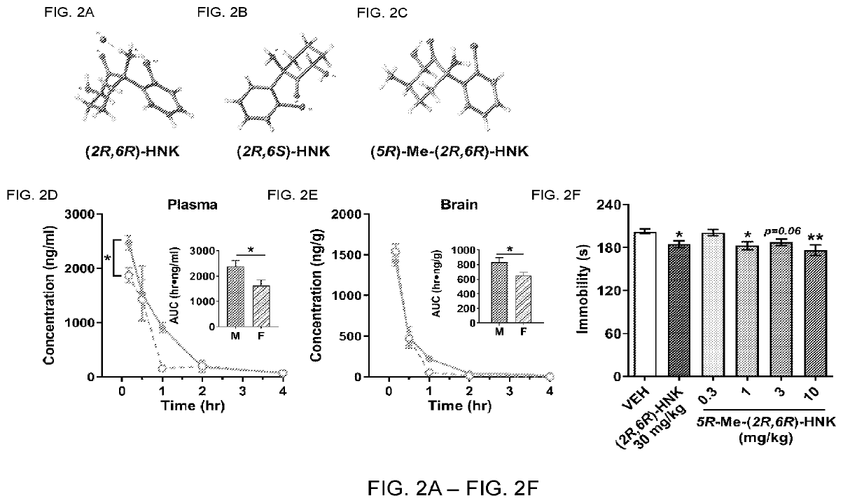

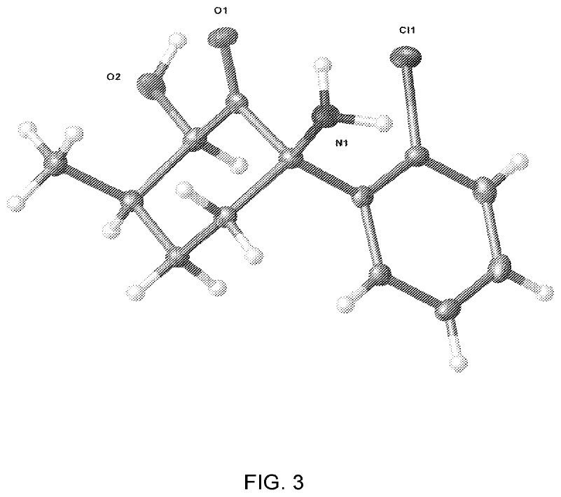

Based upon the finding that (2R,6S)-HNK reduced forced swim test immobility with greater effectiveness compared to (2R,6R)-HNK (FIGS. 1A-1D), it was hypothesized that differences in the three-dimensional structures of these compounds (FIGS. 2A-2B) underlie their differential behavioral effectiveness. While in (2R,6R)-HNK the amine and hydroxyl groups are preferentially oriented equatorial and the aryl group axial to the cyclohexyl ring (FIG. 2A), in (2R,6S)-HNK the aryl group is preferentially oriented equatorial to the cyclohexyl ring (FIG. 2B). Thus, the novel compound (5R)-methyl-(2R,6R)-HNK was synthesized, where the methyl group substitution causes the thermodynamic equilibrium of the compound to adopt a three-dimensional structure more akin to (2R,6S)-HNK (FIG. 2C). Because it was hypothesized that (5R)-methyl-(2R,6R)-HNK would exert antidepressant-relevant behavioral effects at low doses, it was tested within the range of 0.3-10 mg/kg. By shifting the thermodynamic equilibrium between the two chair confirmations to one that more closely resembles that of (2R,6S)-HNK, it was predicted that the (5R)-methyl-(2R,6R)-HNK would have antidepressant effectiveness similar to (2R,6S)-HNK.

(5R)-methyl-(2R,6R)-HNK was tested within the range of 0.3-10 mg/kg. Similar to (2R,6S)-HNK, (5R)-methyl-(2R,6R)-HNK reduced immobility time in the forced swim test with a minimum effective dose of 1 mg/kg, and was effective within the range of 1-10 mg/kg (with a trend to reduce immobility, p=0.06, observed at the dose of 3 mg/kg; FIG. 2D). The present data demonstrate that the novel compound (5R)-methyl-(2R,6R)-HNK exerts antidepressant-relevant behavioral effects in the forced swim test, (FIG. 2F) with similar effectiveness compared to (2R,6S)-HNK (FIG. 1C), consistent with the hypothesis that the three-dimensional structure of (2R,6S)-HNK confers its enhanced effectiveness compared to (2R,6R)-HNK.

### Discussion

For the first time, antidepressant-relevant behavioral effects of the four (2,6)-HNKs were compared in the mouse forced swim test. Results presented here demonstrate that (2R,6R)-HNK has greater behavioral effectiveness (minimum effective dose 10 mg/kg, i.p.; FIG. 1A) compared to (2S,6S)-HNK (minimum effective dose 30 mg/kg, i.p.; FIG. 2B). This is consistent with an earlier study, which reported that (2R,6R)-HNK reduced forced swim test immobility time at doses as low as 5 mg/kg, i.p. and reduced escape failures in the learned helplessness test at doses as low as 3 mg/kg, i.p., whereas (2S,6S)-HNK required doses of 25 and 75 mg/kg, i.p., respectively, to exert effects in the same behavioral tests. This is particularly striking, considering that following systemic treatment with equivalent doses, Cₘₐₓ and AUC of (2S,6S)-HNK are approximately 2-3-fold greater than those of (2R,6R)-HNK in male mice (see Table 8). Although not wishing to be bound by any particular theory, considering these pharmacokinetic differences, it is likely that (2R,6R)-HNK is substantially more potent than (2S,6S)-HNK with respect to engaging its targets in the brain.

The relatively greater behavioral effectiveness of (2R,6R)-HNK compared to that of (2S,6S)-HNK is also consistent with earlier studies demonstrating that, when tested at equivalent doses (10 mg/kg, i.p.), (2R,6R)-, but not (2S,6S)-HNK, reduced forced swim test immobility time in mice and reversed learned helplessness in rats. However, these findings are in contrast to one study which reported that (2S,6S)-, but not (2R,6R)-HNK, reduced forced swim test immobility time and measures of anhedonia following chronic stress in mice when each was tested at the same dose. It is unclear whether differences in experimental design, species, or strain may underlie these apparent discrepancies. Nevertheless, by testing across a 100-fold range of doses and comparing compounds under the same experimental conditions, the present data support that (2R,6R)-HNK exhibits greater behavioral effectiveness, relative to (2S,6S)-HNK, in the forced swim test.

A novel finding is that both (2R,6S)-HNK (effective at 1 mg/kg, i.p.; FIG. 1C) and (2S,6R)-HNK (minimum effective dose 3 mg/kg, i.p.; FIG. 1D) exerted behavioral effects in the forced swim test at lower doses compared to either (2R,6R)- or (2S,6S)-HNK. Altogether, the rank-order of effectiveness in the forced swim test was determined to be (2R,6S)-, (2S,6R)-, (2R,6R)-, and (2S,6S)-HNK, from most to least effective. Although these findings await further validation, these data are the first to highlight the potential superior potency of (2R,6S)-HNK.

The novel compound (5R)-methyl-(2R,6R)-HNK adopts a three-dimensional structure similar to (2R,6S)-HNK (FIGS. 2B-2C). In particular, the methyl group substitution at the C5 position causes (5R)-methyl-(2R,6R)-HNK to localize the aryl group equatorial to the cyclohexyl ring while the amine and hydroxyl groups are localized axial, similar to (2R,6S)-HNK (FIG. 2B) and in contrast with (2R,6R)-HNK (FIG. 2A). Notably, (5R)-methyl-(2R,6R)-HNK reduced immobility time (minimum effective dose 1 mg/kg, i.p.; FIG. 2F) with similar effectiveness as (2R,6S)-HNK, consistent with the hypothesis that the three-dimensional structure of (2R,6S)-HNK confers enhanced behavioral effectiveness. It is unclear whether (5R)-methyl-(2R,6R)-HNK will share the U-shaped dose response curve observed for (2R,6S)-HNK when tested at doses >30 mg/kg. Additionally, the range of doses tested did not allow the determination of a true minimally effective dose of (2R,6S)-HNK (which reduced immobility at the lowest dose tested, 1 mg/kg). Thus, the dose-response of both of these compounds are more fully characterized to determine whether (5R)-methyl-(2R,6R)-HNK recapitulates the complete dose response characteristics of (2R,6S)-HNK.

There are several important limitations to the present study. First, only one behavioral outcome, immobility time in the forced swim test, was evaluated and only at a single time point. It is possible that the relative effectiveness of the (2,6)-HNKs may differ in additional behavioral tests thought to be predictive of antidepressant efficacy or at different times relative to testing (e.g. it is possible that some HNKs may have longer-lasting actions or exert more robust effects when evaluated earlier or later relative to dosing). It is important for future studies to evaluate the effects of the (2,6)-HNKs in a variety of behavioral tests, including those that predict antidepressant effectiveness and in those that aim to characterize adverse behavioral effects. Second, only male mice were included in these experiments, in order to evaluate the relative effectiveness of the test compounds independent of the previously observed sex-dependent differences in their brain concentrations.

Of note, total brain concentrations of (2R,6R)-, (2S,6S)-, and (2S,6R)-HNK were greater in female mice compared to males, while total brain concentrations of (2R,6S)-HNK were greater in male than female mice.

In addition to the sex-dependent differences in their pharmacokinetics profiles, it is important to note that within-sex differences between the four (2,6)-HNKs have been observed, including differences in their peak and total brain concentrations. In particular, following systemic treatment with equivalent doses, peak and total brain levels of (2S,6S)-HNK are approximately 2-3-fold greater than those of (2R,6R)-HNK in male mice. However, despite its greater brain concentrations, (2S,6S)-HNK was less effective in reducing immobility time in the forced swim test, requiring three-fold higher doses to exert effects, compared to (2R,6R)-HNK (minimum effective doses of 30 vs. 10 mg/kg, i.p., respectively; FIGS. 1A-1B). Thus, in light of the observed pharmacokinetic differences, it is possible that (2R,6R)-HNK is substantially more potent than (2S,6S)-HNK with respect to its engagement of targets in the brain. By the same token, it is possible that (2R,6S)- and (2S,6R)-HNK are even more potent compared to (2R,6R)-HNK as suggested by FIGS. 2A-2F, since both compounds exhibit greater behavioral effectiveness (effective at 1 mg/kg, i.p. and 3 mg/kg, i.p., respectively), despite reaching lower brain concentrations. Namely, peak and total brain levels were approximately two-fold lower following (2R,6S)-HNK dosing and approximately three-fold lower following (2S,6R)-HNK dosing, compared to equivalent dosing with (2R,6R)-HNK.

Without wishing to be bound by any particular theory, it is believed that differences in three-dimensional structure of HNKs confer distinct potencies to engage targets underlying their behavioral effects, and several putative targets have been identified. Specifically, (2R,6R)-HNK has been shown to potentiate glutamatergic transmission via a facilitation of glutamate release. HNK-induced facilitation glutamatergic transmission has also been associated with subsequent activation of intracellular signaling pathways, including the BDNF/TrkB and mTOR pathways, which can lead to increased expression of synaptic proteins including AMPARs that ultimately promote a long-lasting increase in synaptic strength in mood-regulating brain regions. Additional studies can evaluate the effects of (2R,6S)-, (2S,6R)-, and (5R)-methyl-(2R,6R)-HNK on these various biochemical and synaptic outcomes, and, importantly, compare their relative potencies to induce such effects, in order to understand: 1) how the potency to engage putative targets is related to the observed rank-order of behavioral effectiveness, and 2) how the three-dimensional structures of the (2,6)-HNKs modulates their effectiveness to engage these putative targets. Additional studies can characterize the behavioral, biochemical, and synaptic actions of the (2,4)- and (2,5)-HNKs, and explain the structure-function relationship underlying the antidepressant-relevant behavioral effects of the HNKs.

Altogether, the rank-order of effectiveness in the forced swim test was determined to be (2R,6S)-, (2S,6R)-, (2R,6R)-, and (2S,6S)-HNK, from most to least effective. It was observed that the range of doses tested did not allow the determination of a true minimally effective dose of (2R,6S)-HNK, which reduced immobility at the lowest dose tested, 1 mg/kg. Nonetheless, the data led to synthesis and characterization of the novel compound (5R)-methyl-(2R,6R)-HNK, which adopts a three-dimensional structure similar to (2R,6S)-HNK (FIGS. 2A-2F). In particular, the methyl group substitution at the C5 position causes (5R)-methyl-(2R,6R)-HNK to localize the aryl group equatorial to the cyclohexyl ring while the amine and hydroxyl groups are localized axial, similar to (2R,6S)-HNK (FIG. 2D) and in contrast with (2R,6R)-HNK (FIG. 2A). Similar to the HNKs formed in vivo from ketamine, (5R)-methyl-(2R,6R)-HNK rapidly penetrated the brain of mice following an intraperitoneal injection, where it was detected at the earliest sampling timepoint, 10 min post-injection. In the forced swim test, (5R)-methyl-(2R,6R)-HNK reduced immobility time (minimum effective dose 1 mg/kg, i.p.; FIG. 2F) with similar effectiveness as (2R,6S)-HNK, consistent with the hypothesis that the three-dimensional structure of (2R,6S)-HNK confers enhanced behavioral effectiveness. It is unclear whether (5R)-methyl-(2R,6R)-HNK will share the U-shaped dose response curve observed for (2R,6S)-HNK when tested at doses ≥30 mg/kg.

The present data are consistent with an NMDAR inhibition-independent mechanism contributing to the actions of the (2,6)-HNKs. In particular, (2S,6S)-HNK has been demonstrated to have moderate inhibitory actions on NMDARs (Kᵢ=7.34-21.19 μM) compared to ketamine (Kᵢ=0.25-1.06 μM). By contrast, (2R,6R)-HNK has markedly lower potency to bind or inhibit NMDARs (Kᵢ>100 μM), and does not meaningfully inhibit NMDARs at concentrations similar to those produced following the behaviorally active dose of 10 mg/kg, i.p. (estimated to be <20 μmol/kg in brain tissue and <10 μM in the extracellular hippocampal space).

The relatively low binding affinities of (2R,6S)- and (2S,6R)-HNK are similar to that of (2R,6R)-HNK (all were reported to be >100 μM). Both compounds reduced immobility time in the forced swim test at doses ≤10 mg/kg, i.p., despite producing lower brain concentrations that similar doses of (2R,6R)-HNK, suggesting, without wishing to be bound by any particular theory, that the brain concentrations of (2R,6S)- and (2S,6R)-HNK necessary to induce antidepressant-like actions are even lower than those required for (2R,6R)-HNK, and, therefore, also below the threshold for NMDAR inhibition. Altogether, these findings suggest that (2R,6S)- and (2S,6R)-HNK exert antidepressant-relevant behavioral actions at brain concentrations that are insufficient to inhibit NMDARs, consistent with an NMDAR-independent mechanism of action.

Altogether, the data presented here demonstrate, for the first time, that the relative rank-order of effectiveness of the four (2,6)-HNKs to reduce forced swim test immobility time, from most to least effective, is (2R,6S)-, (2S,6R)-, (2R,6R)-, and (2S,6S)-HNK. This is the first indication that (2R,6S)-HNK has greater effectiveness compared to (2R,6R)-HNK, exerting antidepressant-relevant behavioral effects at 10-fold lower doses, despite its lower relative brain concentrations. Consistent with the three-dimensional structure of (2R,6S)-HNK conferring its enhanced effectiveness, the structurally similar, novel compound (5R)-methyl-(2R,6R)-HNK recapitulated the effectiveness of (2R,6S)-HNK. Without wishing to be bound by any particular theory, the data suggest that (2R,6S)-HNK and (5R)-methyl-(2R,6R)-HNK may represent novel, potent drug candidates for the treatment of depression.

### Conclusion

The studies presented here aimed to address several important considerations for the development of HNKs as novel antidepressant treatments. First, the pharmacokinetic profiles of the 12 HNKs formed in vivo from ketamine were characterized. Robust differences in peak concentrations (6-fold differences in the plasma, 7-8-fold differences in the brain) and total levels (11-12-fold differences in the plasma, 9-12-fold differences in the brain) in the plasma and the brain were observed between the various HNKs, in addition to sex-dependent differences in peak and total concentrations that were identified for most HNKs. However, all 12 HNKs readily penetrated the brain (with brain to plasma exposure ratios between 0.6-1.4) and shared similar rapid elimination profiles (half-lives between 0.4-0.8 h in the plasma and 0.3-0.8 h in the brain). These data provide the first in-depth assessment of the pharmacokinetics of the 12 HNKs, and, importantly, highlight the ability of all 12 HNKs to readily penetrate the brain, suggesting that these compounds may be capable of exerting pharmacodynamic effects in the brain, including antidepressant-relevant actions.

Second, it was demonstrated that (2R,6R)-HNK has favorable oral bioavailability of approximately 50% (between 45-52%) in mice, which was not improved by an ester prodrug strategy. Notably, orally administered (2R,6R)-HNK exerted antidepressant-relevant behavioral actions (15-50 mg/kg) but did not exert overt adverse effects (up to 450 mg/kg, at least 9-fold fold higher than doses required for antidepressant-relevant effects) in mice. Taken together, these data may support the use of (2R,6R)-HNK as an oral antidepressant drug, although its antidepressant efficacy and oral bioavailability in humans awaits testing in clinical trials.

Additionally, sex-dependent differences in the pharmacokinetics of ketamine and (2R,6R)-HNK were characterized. It was demonstrated that, compared to males, female mice have lower levels of ketamine and higher levels of HNK following ketamine administration, as well as higher levels of (2R,6R)-HNK following its direct administration, and that male gonadal hormones at least partly mediate these differences. Although not wishing to be bound by any particular theory, while the precise mechanisms by which male gonadal hormones modulate metabolism and the impact of these metabolic differences on the behavioral effectiveness of ketamine and/or (2R,6R)-HNK require further clarification, the observed sex-dependent pharmacokinetic differences may contribute to differences in antidepressant efficacy and/or adverse effect burden between the sexes, and highlight the importance of sex as a biological variable when designing and interpreting studies of ketamine and (2R,6R)-HNK.

Further, the relative behavioral effectiveness of the four (2,6)-HNKs was evaluated in the mouse forced swim test in male mice. The data demonstrated for the first time that both (2R,6S)-HNK (effective at 1 mg/kg, i.p.) and (2S,6R)-HNK (minimum effective dose 3 mg/kg, i.p.) exhibit greater effectiveness compared to either (2R,6R)-HNK (minimum effective dose 10 mg/kg, i.p.) or (2S,6S)-HNK (minimum effective dose 30 mg/kg, i.p.). They also indicated a relative rank-order of effectiveness of (2R,6S)->(2S,6R)->(2R,6R)->(2S,6S)-HNK to reduce immobility time in the forced swim test. Interesting, enhanced behavioral effectiveness of (2R,6S)-HNK compared to (2S,6R)-HNK was observed despite (2R,6S)-HNK dosing resulting in lower peak and total brain concentrations compared to equivalent dosing of (2R,6R)-HNK. Although not wishing to be bound by any particular theory, these results suggest that, if these compounds were tested at equal concentrations and compared independent of pharmacokinetic differences (for example testing in in vitro models), (2R,6S)-HNK may demonstrate an even more robust enhancement in potency to engage targets compared to (2R,6R)-HNK. However, this possibility requires further testing in future studies.

The novel compound (5R)-methyl-(2R,6R)-HNK, which shares a similar three-dimensional structure with (2R,6S)-HNK, recapitulated the behavioral effectiveness of (2R,6S)-HNK in the forced swim test, suggesting, without wishing to be bound by any particular theory, a critical role of three-dimensional structure in mediating antidepressant-relevant effectiveness in this assay, and the data suggest that (2R,6S)-HNK and (5R)-methyl-(2R,6R)-HNK, may represent novel, potent, antidepressant drug candidates. Notably, it is possible that (2R,6S)-HNK and/or (5R)-methyl-(2R,6R)-HNK may have greater potency to engage targets involved in mediating their antidepressant-relevant behavioral effects.

Additional studies can (i) identify the mechanisms underlying the antidepressant-relevant behavioral actions observed here, (ii) evaluate the full range of behavioral actions including potential adverse effects of the (2,6)-HNKs, and (iii) identify the mechanisms underlying sex-dependent differences in the pharmacokinetic profile of ketamine and HNKs and how these differences impact behavioral outcomes.

The present studies provide important insights into the pharmacokinetic profiles and behavioral effects of the HNKs, particularly, the (2,6)-HNKs, and lay the foundation for additional studies to elucidate the potential clinical relevance of HNKs as novel antidepressant treatments.

### Example 3: Pharmacokinetics and Antidepressant-Relevant Effects of the (2,6)- and (5R)-methyl-(2R,6R)-hydroxynorketamines

This Example relates to metabolites of (R,S)-ketamine. (R,S)-ketamine is rapidly metabolized to form a range of metabolites in vivo, including 12 unique hydroxynorketamines (HNKs) distinguished by the site of cyclohexyl ring hydroxylation at the 4, 5, or 6 position. While both (2R,6R)- and (2S,6S)-HNK readily penetrate the brain and exert rapid antidepressant-like actions in preclinical tests following peripheral administration, the pharmacokinetic profiles and pharmacodynamic actions of the ten other HNKs have not been examined. It was hypothesized that distinct structure-activity relationships would define the relative potencies of the HNKs and increased potency when the aryl group is located equatorial to the cyclohexyl ring and the amine and hydroxyl groups are localized axial resulting in synthesis of (5R)-methyl-(2R,6R)-HNK. The pharmacokinetic profiles of all 12 HNKs was assessed in the plasma and brains of male and female mice and compared the relative effectiveness of the four (2,6)-HNKs to induce antidepressant-relevant behavioral effects in the forced swim test in male mice. While all HNKs were readily brain-penetrable, there were robust differences in peak plasma and brain concentrations and exposures.

### Materials and Methods

### Animals

Male and female CD-1 mice (Charles Rivers Laboratories, Raleigh, NC, USA), 8-11 weeks old at the time of testing, were habituated to the University of Maryland (Baltimore, MD, USA) animal facility for at least one week prior to testing. Mice were group housed 4-5 per cage with a constant 12-hour light cycle (lights on/off at 07:00/19:00). Food and water were available ad libitum. All experiments were performed during the light phase. All animal studies were approved by the University of Maryland School of Medicine Institutional Animal Care and Use Committee and conducted in accordance with the National Institutes of Health Guide for the Care and Use of Laboratory Animals.

### Drugs

(2R,6R)-HNK hydrochloride, (2S,6S)-HNK hydrochloride, (2R,6S)-HNK, (2S,6R)-HNK, (2R,5R)-HNK, (2S,5S)-HNK, (2R,5S)-HNK, (2S,5R)-HNK, (2R,4R)-HNK, (2S,4S)-HNK, (2R,4S)-HNK, and (2S,4R)-HNK hydrochloride were synthesized at the National Center for Advancing Translation Sciences (National Institutes of Health, Rockville, MD, USA) using methods previously described in Morris et al., Org Lett 17:4572-4575 (2017), which is incorporated by reference herein in its entirety. (5R)-methyl-(2R,6R)-HNK was obtained from Organix Inc. (Woburn, MA, USA) or from the National Center for Advancing Translational Sciences (Rockville, MD, USA); synthesis and characterization are described in the Supplemental Information. All compounds were dissolved in 20% (w/v) cyclodextrin (Sigma Aldrich, USA) in saline and administered intraperitoneally (i.p.) in a volume of 7.5 ml/kg.

### Pharmacokinetic Studies

### Dosing and Sample Collection

HNKs were administered intraperitoneally (i.p.) at a dose of 5 mg/kg (the free base dose for compounds provided as a hydrochloride salt—(2R,6R)-HNK, (2S,6S)-HNK, and (2S,4R)-HNK—is equivalent to 4.31 mg/kg; (5R)-methyl-(2R,6R)-HNK is equivalent to 4.37 mg/kg) and a volume of 7.5 ml/kg, in separate cohorts of male and female mice (n=4 per sex, compound, and collection time point). At 10 min (0.167 h), 30 min (0.5 h), 1 h, 2 h, and 4 h post-treatment, mice were deeply anesthetized (3.5% isoflurane for approximately 2 min). Trunk blood was collected into 1.5-ml polypropylene tubes containing 30 μl of disodium EDTA (0.5 M, pH 8.0) and kept on ice until plasma collection (<30 min). Brains were then removed, flash-frozen in isopentane, and stored at −80° C. until analysis. Blood was centrifuged at 8000×g for 6 min at 4° C. to obtain plasma. Plasma was collected into clean microcentrifuge tubes and stored at −80° C. until analysis.

### UPLC-MS/MS Analysis of Plasma and Brain Samples

Ultra-performance liquid chromatography-tandem mass spectrometry (UPLC-MS/MS) methods were developed and optimized to determine the concentration of each HNK in mouse plasma and brain samples following systemic administration. Mass spectrometric analysis was performed on a Waters Xevo TQ-S triple quadrupole instrument using electrospray ionization in positive mode with the selected reaction monitoring. The separation of HNKs from endogenous components was performed on a Waters Acquity UPLC with 0.5 ml/min flow rate and gradient elution. The mobile phase A and B were 0.1% formic acid in water and 0.1% formic acid in acetonitrile, respectively. Plasma and brain concentrations of (2R,6R)-, (2S,6S)-, (2R,4S)-, and (2S,4R)-HNK were determined by hydrophilic interaction liquid chromatography-tandem mass spectrometry (HILIC UPLC-MS/MS) using a BEH amide column (1.7μ, 2.1×50 mm). Plasma and brain concentrations of (2R,6S)-, (2S,6R)-, (2R,5R)- (2S,5S)-, (2R,5S)-, (2S,5R)-, (2R,4R)-, and (2S,4S)-HNK; and (5R)-methyl-(2R,6R)-HNK were determined by reversed phase UPLC-MS/MS based upon a previously described protocol with several modifications using a BEH C₁₈ column (1.7 μM, 2.1×50 mm). See Moaddel et al., Talanta 82:1892-1904 (2010), and Zanos et al., Nature 533:481-486 (2010), both of which are incorporated by reference herein in their entireties.

The gradient for HILIC UPLC method was: 0-0.2 min 98% B; 0.2-1.4 min 98% to 35% B; 1.4-1.8 min 35% to 10% B, 1.9-2.4 min 98% B. The gradient for BEH Cis UPLC method was: 0-0.2 min 1% B; 0.2-1.4 min 1% to 35% B; 1.4-1.8 min 35% to 98% B, 1.8-2.2 min 98% B, 2.3 min 1% B. The analytical run time of the UPLC methods was 2.5 min. The calibration standards and quality control samples were prepared in blank mouse plasma and brain homogenate. Calibration standards ranged from 5.0-5000 ng/ml. A linear regression with 1/x² weighting was used to construct the calibration curve. Quality control samples were prepared at 10.0 ng/ml, 100 ng/ml, and 2500 ng/ml in the corresponding matrix. Brains were weighed and homogenized in 3 volumes of water. d4-HNK was used as internal standard. 200 μL of 50 ng/ml d4-HNK solution in acetonitrile was used to precipitate proteins. The supernatant was transferred for UPLC-MS/MS analysis. The data were acquired using MassLynx and analyzed using TargetLynx version 4.1. The lower limit of quantification (LLOQ) was 5.0 ng/ml.

### Pharmacokinetic Analysis

The pharmacokinetic parameters of (2R,6R)- (2S,6S)-, (2R,6S)-, (2S,6R)-, (2R,5R)-(2S,5S)-, (2R,5S)-, (2S,5R)-, (2R,4R)- (2S,4S)-, (2R,4S)-, (2S,4R), and (5R)-methyl-(2R,6R)-HNK were calculated using non-compartmental analysis (Model 200) in the pharmacokinetic software Phoenix WinNonlin (version 7.0, Certara, St. Louis, MO). The area under the plasma and tissue concentration versus time curve (AUC) was calculated using the linear trapezoidal method. The slope of the elimination phase was estimated by log linear regression using at least 3 data points and the terminal rate constant (k) was derived from the slope. AUC_{0→∞} was calculated as the sum of the AUC_{0→t} (where t is the time of the last measurable concentration) and Cₜ/λ. The apparent elimination half-life (t_{1/2}) was calculated as 0.693/λ.

### Statistical Analyses

Statistical analyses were performed using GraphPad Prism software (v8; GraphPad Software, Inc.). In cases of two-group comparisons, all statistical tests were two-tailed, and significance was assigned at p<0.05. Significant results are indicated with asterisks in the figures (*p<0.05, **p<0.01, ***p<0.001). Data are presented as the mean±standard error of the mean (SEM). Mice were randomized to treatment groups and experiments were performed in a blinded manner. For the pharmacokinetic analysis of HNK, sex-dependent differences in concentrations over time were analyzed using a two-way ANOVA with sex and time as factors, followed by Holm-Šídák post-hoc comparisons when significant main effects were observed. The experimental design (in which plasma and brain for a single experimental time point came from independent groups of mice, representing a between-group comparisons as a function of time) resulted in a single experimentally derived AUC per compound and sex. Thus, to enable the comparison of AUCs between sexes, a previously described bootstrapping method was utilized to estimate the mean and SEM for each AUC. See Shen and Machado, Biopharm Stat 27:257-264 (2017), which is incorporated by reference herein in its entirety. Briefly, to calculate the mean and SEM of the AUC, N=1000 of 1024 possible combinations were randomly selected to form N time series, and then 1000 AUCs and the corresponding mean and SEM determined for each sex. Sex-dependent differences in AUCs were determined using a standard t-test. For the behavioral testing of HNKs in the forced-swim test, differences in immobility time were analyzed via one-way ANOVA followed by Holm-Šídák post-hoc comparisons (with all groups compared to the saline control group) when a significant main effect of the model was observed.

### Results

### Pharmacokinetic Profiles of the 12 HNKs in Mouse Plasma and Brain

### Plasma Concentrations of HNKs

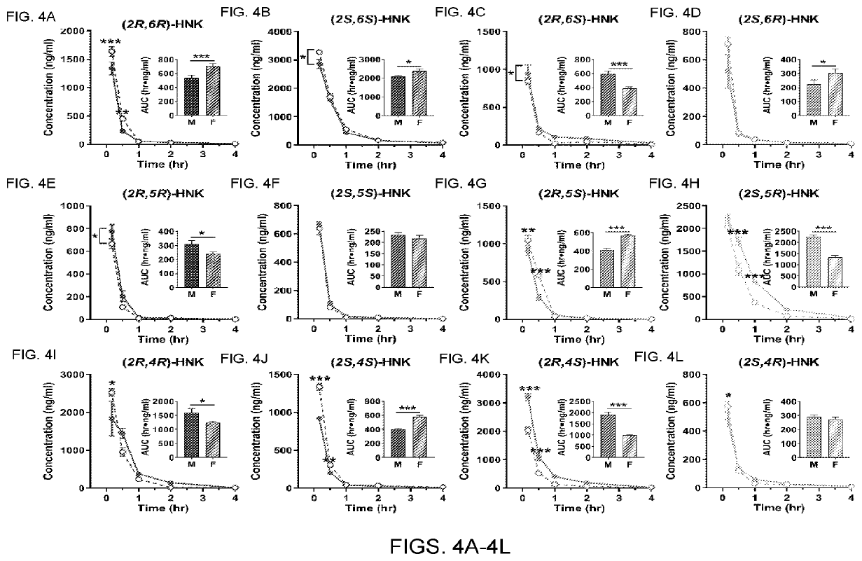

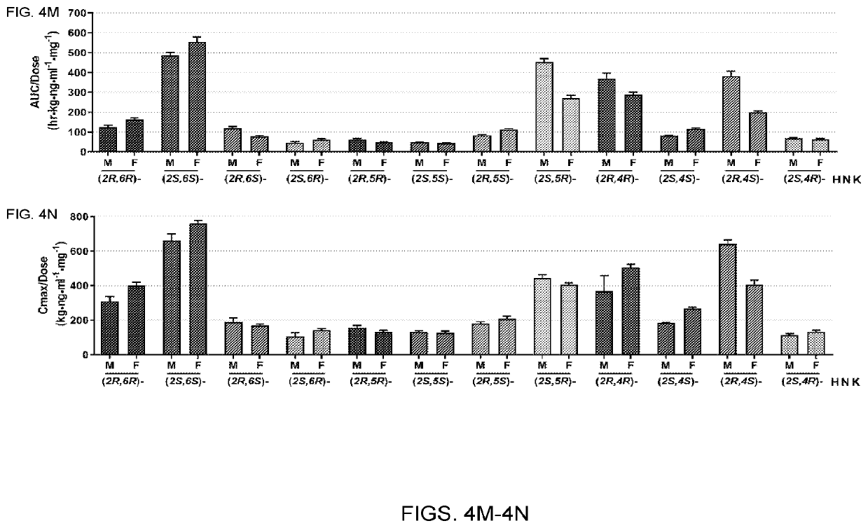

All HNKs were detected in the plasma of male and female mice between 10 min (earliest time point tested) and 4 h (latest time point tested) after i.p. injection. Peak plasma levels (Cₘₐₓ) for all HNKs were observed in both sexes at the earliest time point, i.e., 10 min post-treatment (FIGS. 4A-4N and Table 1). Robust differences for effects of sex and compound were observed in both plasma Cₘₐₓ and AUC (Table 8). Approximately 6-fold differences were observed between the dose-normalized plasma Cₘₐₓ of HNKs in both sexes, with the highest levels observed for (2S,6S)-HNK in both sexes, and the lowest observed for (2S,6R)-HNK in males and (2S,5S)-HNK in females (Table 8). Approximately 11- and 12-fold differences were observed in the dose-normalized HNK AUC among male and female mice, respectively (Table 8). Consistent with the trends noted for Cₘₐₓ levels, the highest AUC were observed following (2S,6S)-HNK injection in male and female mice, while the lowest AUC were observed for (2S,6R)-HNK in males and (2S,5S)-HNK in females (Table 8). Plasma levels decreased rapidly after dosing and plasma elimination half-lives ranged from 0.4-0.8 h in females and from 0.5-0.9 h in males (Table 8).

TABLE 8
Hydroxynorketamine pharmacokinetics in male and female mice^{a}.

| | | Cₘₐₓ^{b} | | AUC^{b} | | | | |
|---|---|---|---|---|---|---|---|---|
| | | (mean ± SEM) | | (mean ± SEM) | | | | |
| | | Plasma | Brain | Plasma | Brain | | t_{1/2} | |
| | | (ng/ml · | (ng/g · | (ng/ml · hr · | (ng/g · hr · | B:P | Plasma | Brain |
| HNK | Sex | mg/kg) | mg/kg) | mg/kg) | mg/kg) | ratio | (hr) | (hr) |
| (2R,6R)-^{c} | Male | 310 ± 26.1 | 380 ± 19.3 | 125 ± 8.6 | 165 ± 13.9 | 1.3 | 0.6 | 0.6 |
| | Female | 401 ± 18.3 | 530 ± 17.2 | 163 ± 7.9 | 220 ± 8.8 | 1.3 | 0.6 | 0.6 |
| (2S,6S)-^{c} | Male | 663 ± 36.4 | 712 ± 47.3 | 485 ± 15.5 | 462 ± 20.6 | 1.0 | 0.7 | 0.7 |
| | Female | 760 ± 17.0 | 993 ± 27.1 | 555 ± 24.1 | 689 ± 30.9 | 1.2 | 0.8 | 0.7 |
| (2R,6S)- | Male | 189 ± 23.0 | 173 ± 5.4 | 119 ± 7.9 | 67.8 ± 2.9 | 0.6 | 0.9 | 0.7 |
| | Female | 169 ± 8.2 | 166 ± 12.3 | 76.9 ± 4.8 | 55.7 ± 3.5 | 0.7 | 0.8 | 0.4 |
| (2S,6R)- | Male | 104 ± 23.9 | 117 ± 30.6 | 44.9 ± 6.1 | 53.4 ± 8.0 | 1.2 | 0.7 | 0.8 |
| | Female | 142 ± 9.4 | 177 ± 19.8 | 61.0 ± 5.2 | 85.6 ± 8.9 | 1.4 | 0.8 | 0.8 |
| (2R,5R)- | Male | 155 ± 12.6 | 142 ± 5.6 | 61.8 ± 5.1 | 73.9 ± 4.5 | 1.2 | 0.5 | 0.4 |
| | Female | 133 ± 7.8 | 152 ± 8.8 | 48.2 ± 2.2 | 69.5 ± 2.8 | 1.4 | 0.5 | 0.5 |
| (28,5S)- | Male | 133 ± 4.2 | 138 ± 8.3 | 47.2 ± 1.8 | 57.1 ± 2.4 | 1.2 | 0.5 | 0.3 |
| | Female | 128 ± 9.1 | 129 ± 2.8 | 43.8 ± 2.7 | 55.0 ± 2.0 | 1.3 | 0.6 | 0.3 |
| (2R,5S)- | Male | 180 ± 10.6 | 106 ± 6.8 | 82.2 ± 4.8 | 70.1 ± 5.1 | 0.9 | 0.5 | 0.5 |
| | Female | 209 ± 13.4 | 154 ± 3.9 | 113 ± 3.8 | 115 ± 4.0 | 1.0 | 0.5 | 0.5 |
| (28,5R)- | Male | 443 ± 20.1 | 242 ± 9.8 | 454 ± 15.4 | 309 ± 14.0 | 0.7 | 0.6 | 0.7 |
| | Female | 405 ± 10.6 | 258 ± 15.4 | 271 ± 14.4 | 259 ± 16.2 | 1.0 | 0.5 | 0.5 |
| (2R,4R)- | Male | 367 ± 92.1 | 431 ± 43.5 | 369 ± 28.2 | 496 ± 38.0 | 1.3 | 0.6 | 0.6 |
| | Female | 505 ± 19.0 | 490 ± 26.4 | 289 ± 11.2 | 358 ± 16.4 | 1.2 | 0.5 | 0.5 |
| (2S,4S)- | Male | 184 ± 1.8 | 127 ± 9.5 | 80.4 ± 3.1 | 57.8 ± 3.8 | 0.7 | 0.7 | 0.6 |
| | Female | 267 ± 8.5 | 234 ± 10.9 | 115 ± 4.8 | 101 ± 4.5 | 0.9 | 0.7 | 0.6 |
| (2R,4S)- | Male | 640 ± 24.7 | 758 ± 21.4 | 382 ± 24.6 | 422 ± 34.6 | 1.1 | 0.6 | 0.5 |
| | Female | 406 ± 25.9 | 495 ± 28.7 | 198 ± 8.0 | 194 ± 9.6 | 1.0 | 0.7 | 0.3 |
| (2S,4R)-^{c} | Male | 112 ± 11.3 | 160 ± 10.2 | 67.4 ± 3.3 | 62.6 ± 3.4 | 0.9 | 0.5 | 0.4 |
| | Female | 133 ± 8.9 | 190 ± 13.7 | 63.3 ± 3.7 | 64.3 ± 3.5 | 1.0 | 0.4 | 0.3 |
| (5R)-Me- | Male | 565 ± 30.2 | 324 ± 13.0 | 544 ± 51.9 | 191 ± 14.4 | 0.4 | 0.8 | 0.8 |
| (2R,6R)- | Female | 427 ± 31.8 | 352 ± 22.4 | 371 ± 51.7 | 149 ± 10.1 | 0.4 | 0.8 | 0.7 |

^{a}In the plasma, Tₘₐₓ was 10 min for all HNKs in both sexes; in the brain, Tₘₐₓ was 10 min for all HNKs in both sexes except for (2R,5S)-HNK (Tₘₐₓ = 10 min in males and 30 min in females) and (2R,4R)-HNK (Tₘₐₓ = 30 min in males and 10 min in females).
^{b}For each Cₘₐₓ or AUC, respectively, the dose is adjusted to account for the free base content of the compound.
^{c}(2R,6R)-, (2S,6S)-, (2S,4R)-HNK, and (5R)-methyl (Me)-(2R,6R)-HNK were administered as a HCl salt (equivalent to free base dose 4.31 mg/kg for (2R,6R)-, (2S,6S)-, (2S,4R)-HNK and 4.37 mg/kg for (5R)-Me-(2R,6R)-HNK); all other HNKs were administered as the free base (5 mg/kg).
Abbreviations: AUC, area under the concentration vs. time curve normalized to free base dose; B:P ratio; brain to plasma AUC ratio; Cₘₐₓ, maximum observed concentration normalized to free base dose; F, female; hr, hours; HNK, hydroxynorketamine; Tₘₐₓ, time of observed maximal concentrations; M, male; t_{1/2}, terminal half-life.

For most HNKs, Cₘₐₓ and AUC were sex-dependent (FIGS. 4A-4N and summarized in Table 9). Female mice had higher Cₘₐₓ and AUC of (2R,6R)-HNK (FIG. 4A), (2S,6S)-HNK (FIG. 4B), (2R,5S)-HNK (FIG. 4G), and (2S,4S)-HNK (FIG. 4J), compared to males that received the same compound at the same dose. Following administration of (2S,6R)-HNK, AUCs were greater in females compared to males, although differences in plasma concentrations at fixed time points did not reach statistical significance (FIG. 4D). Conversely, following administration of (2S,4R)-HNK, plasma concentrations were greater in females than males at 10 min post-injection, but total exposures were not different between the sexes (FIG. 4L). While plasma concentrations were higher during the first 10 min following (2R,4R)-HNK treatment in females than males, AUC were greater in males (FIG. 4I). Compared to females, male mice had higher Cₘₐₓ and AUC of (2R,6S)-HNK (FIG. 4C), (2R,5R)-HNK (FIG. 4E), (2S,5R)-HNK (FIG. 4H), and (2R,4S)-HNK (FIG. 4K). No sex-dependent differences in Cₘₐₓ and AUC were detected following treatment with (2S,5S)-HNK (FIG. 4F).

TABLE 9
Sex-dependent differences in concentrations of HNKs.

| | Plasma | | Brain | |
|---|---|---|---|---|
| | | Total | | Total |
| | | exposure | | exposure |
| HNK | Concentration over time | (AUC) | Concentration over time | (AUC) |
| (2R,6R)- | F > M at 10, 30 min | F > M | F > M at 10, 30 min | F > M |
| | One-way ANOVA: | | One-way ANOVA: | |
| | effect of sex, F(1, 30) = 375.4, | | effect of sex, F(1, 30) = 35.06, | |
| | p = 0.0013 | | p < 0.0001 | |
| | effect of time, F(4, 30) < 0.0001, | | effect of time, F(4, 30) = 746.7, | |
| | p < 0.0001 | | p < 0.0001 | |
| | interaction, F(4, 30) = 4.958, | | interaction, F(4, 30) = 17.99, | |
| | p = 0.0034 | | p < 0.0001 | |
| | post-hoc comparison, effect of | | post-hoc comparison, effect of | |
| | sex at: | | sex at: | |
| | 10 min, p = 0.0004 | | 10 min, p < 0.0001 | |
| | 30 min, p = 0.0086 | | 30 min, p < 0.0001 | |
| | 1 hr, p > 0.9999 | | 1 hr, p = 0.7458 | |
| | 2 hr, p > 0.9999 | | 2 hr, p = 0.9984 | |
| | 4 hr, p > 0.9999 | | 4 hr, p = 0.9984 | |
| (2S,6S)- | main effect of sex, F > M | F > M | F > M at 10, 30 min | F > M |
| | One-way ANOVA: | | One-way ANOVA: | |
| | effect of sex, F(1, 28) = 7.018, | | effect of sex, F(1, 29) = 41.43, | |
| | p = 0.0131 | | p < 0.0001 | |
| | effect of time, F(4, 28) = 503.6, | | effect of time, F(4, 29) = 370.7, | |
| | p < 0.0001 | | p < 0.0001 | |
| | interaction, F(4, 28) = 2.173, | | interaction, F(4, 29) = 9.878, | |
| | p = 0.0980 | | p < 0.0001 | |
| | | | post-hoc comparison, effect of | |
| | | | sex at: | |
| | | | 10 min, p < 0.0001 | |
| | | | 30 min, p = 0.0005 | |
| | | | 1 hr, p = 0.1921 | |
| | | | 2 hr, p = 0.9494 | |
| | | | 4 hr, p = 0.9494 | |
| (2R,6S)- | main effect of sex, M > F | M > F | NS | M > F |
| | One-way ANOVA: | | One-way ANOVA: | |
| | effect of sex, F(1, 30) = 5.136, | | effect of sex, F(1, 30) = 0.7799, | |
| | p = 0.0308 | | p = 0.3842 | |
| | effect of time, F(4, 30) = 160.4, | | effect of time, F(4, 30) = 516.9, | |
| | p < 0.0001 | | p < 0.0001 | |
| | interaction, F(4, 30) = 0.3086, | | interaction, F(4, 30) = 0.1980, | |
| | p = 0.0308 | | p = 0.9375 | |
| (2S,6R)- | NS | F > M | main effect of sex, F > M | F > M |
| | One-way ANOVA: | | One-way ANOVA: | |
| | effect of sex, F(1, 29) = 2.413, | | effect of sex, F(1, 30) = 5.109, | |
| | p = 0.1312 | | p = 0.0312 | |
| | effect of time, F(4, 29) = 77.73, | | effect of time, F(4, 30) = 27.44, | |
| | p < 0.0001 | | p < 0.0001 | |
| | interaction, F(4, 29) = 2.034, | | interaction, F(4, 30) = 1.153, | |
| | p = 0.1157 | | p = 0.3512 | |
| (2R,5R)- | main effect of sex, M > F | M > F | NS | NS |
| | One-way ANOVA: | | One-way ANOVA: | |
| | effect of sex, F(1, 30) = 5.404, | | effect of sex, F(1, 30) = 0.2341, | |
| | p = 0.0270 | | p = 0.6320 | |
| | effect of time, F(4, 30) = 239.4, | | effect of time, F(4, 30) = 359.1, | |
| | p < 0.0001 | | p < 0.0001 | |
| | interaction, F(4, 29) = 1.912, | | interaction, F(4, 30) = 2.149, | |
| | p = 0.1342 | | p = 0.0991 | |
| (2S,5S)- | NS | NS | NS | NS |
| | One-way ANOVA: | | One-way ANOVA: | |
| | effect of sex, F(1, 30) = 0.9101, | | effect of sex, F(1, 30) = 0.5869, | |
| | p = 0.3477 | | p = 0.4496 | |
| | effect of time, F(4, 30) = 516.8, | | effect of time, F(4, 30) = 613.1, | |
| | p < 0.0001 | | p < 0.0001 | |
| | interaction, F(4, 30) = 0.2707, | | interaction, F(4, 30) = 0.8868, | |
| | p = 0.8945 | | p = 0.4838 | |
| (2R,5S)- | F > M at 10, 30 min | F > M | F > M at 10, 30 min | F > M |
| | One-way ANOVA: | | One-way ANOVA: | |
| | effect of sex, F(1, 30) = 19.25, | | effect of sex, F(1, 30) = 42.93, | |
| | p = 0.0001 | | p < 0.0001 | |
| | effect of time, F(4, 30) = 370.6, | | effect of time, F(4, 30) = 229.2, | |
| | p < 0.0001 | | p < 0.0001 | |
| | interaction, F(4, 30) = 9.187, | | interaction, F(4, 30) = 20.08, | |
| | p < 0.0001 | | p < 0.0001 | |
| | post-hoc comparison, effect of | | post-hoc comparison, effect of | |
| | sex at: | | sex at: | |
| | 10 min, p = 0.0090 | | 10 min, p = 0.0003 | |
| | 30 min, p < 0.0001 | | 30 min, p < 0.0001 | |
| | 1 hr, p = 0.9781 | | 1 hr, p = 0.9923 | |
| | 2 hr, p = 0.9907 | | 2 hr, p = 0.9923 | |
| | 4 hr, p = 0.9907 | | 4 hr, p = 0.9923 | |
| (2S,5R)- | M > F at 30, 60 min | M > F | NS | M > F |
| | One-way ANOVA: | | One-way ANOVA: | |
| | effect of sex, F(1, 30) = 49.49, | | effect of sex, F(1, 30) = 0.2874, | |
| | p < 0.0001 | | p = 0.5959 | |
| | effect of time, F(4, 30) = 377.0, | | effect of time, F(4, 30) = 149.5, | |
| | p < 0.0001 | | p < 0.0001 | |
| | interaction, F(4, 30) = 7.273, | | interaction, F(4, 30) = 2.214, | |
| | p = 0.0003 | | p = 0.0913 | |
| | post-hoc comparison, effect of | | | |
| | sex at: | | | |
| | 10 min, p = 0.1424 | | | |
| | 30 min, p < 0.0001 | | | |
| | 1 hr, p < 0.0001 | | | |
| | 2 hr, p = 0.2335 | | | |
| | 4 hr, p = 0.7899 | | | |
| (2R,4R)- | F > M at 10 min | M > F | F > M at 10 min | M > F |
| | One-way ANOVA: | | One-way ANOVA: | |
| | effect of sex, F(1, 30) = 0.02068, | | effect of sex, F(1, 30) = 0.2419, | |
| | p = 0.8866 | | p = 0.6264 | |
| | effect of time, F(4, 30) = 67.30, | | effect of time, F(4, 30) = 52.31, | |
| | p < 0.0001 | | p < 0.0001 | |
| | interaction, F(4, 30) = 3.643, | | interaction, F(4, 30) = 4.156, | |
| | p = 0.0156 | | p = 0.0085 | |
| | post-hoc comparison, effect of | | post-hoc comparison, effect of | |
| | sex at: | | sex at: | |
| | 10 min, p = 0.0239 | | 10 min, p = 0.0214 | |
| | 30 min, p = 0.1483 | | 30 min, p = 0.0835 | |
| | 1 hr, p = 0.9036 | | 1 hr, p = 0.7408 | |
| | 2 hr, p = 0.9036 | | 2 hr, p = 0.7408 | |
| | 4 hr, p = 0.9670 | | 4 hr, p = 0.9480 | |
| (2S,4S)- | F > M at 10, 30 min | F > M | F > M at 10, 30 min | F > M |
| | One-way ANOVA: | | One-way ANOVA: | |
| | effect of sex, F(1, 30) = 75.48, | | effect of sex, F(1, 30) = 66.74, | |
| | p < 0.0001 | | p < 0.0001 | |
| | effect of time, F(4, 30) = 1196, | | effect of time, F(4, 30) = 393.2, | |
| | p < 0.0001 | | p < 0.0001 | |
| | interaction, F(4, 30) = 41.32, | | interaction, F(4, 30) = 34.58, | |
| | p < 0.0001 | | p < 0.0001 | |
| | post-hoc comparison, effect of | | post-hoc comparison, effect of | |
| | sex at: | | sex at: | |
| | 10 min, p < 0.0001 | | 10 min, p < 0.0001 | |
| | 30 min, p = 0.0035 | | 30 min, p = 0.0016 | |
| | 1 hr, p = 0.9900 | | 1 hr, p = 0.9785 | |
| | 2 hr, p = 0.9900 | | 2 hr, p = 0.9891 | |
| | 4 hr, p = 0.9900 | | 4 hr, p = 0.9891 | |
| (2R,4S)- | M > F at 10, 30 min | M > F | M > F at 10, 30, 60 min | M > F |
| | One-way ANOVA: | | One-way ANOVA: | |
| | effect of sex, F(1, 30) = 79.77, | | effect of sex, F(1, 30) = 64.71, | |
| | p < 0.0001 | | p < 0.0001 | |
| | effect of time, F(4, 30) = 399.2, | | effect of time, F(4, 30) = 356.8, | |
| | p < 0.0001 | | p < 0.0001 | |
| | interaction, F(4, 30) = 18.95, | | interaction, F(4, 30) = 13.86, | |
| | p < 0.0001 | | p < 0.0001 | |
| | post-hoc comparison, effect of | | post-hoc comparison, effect of | |
| | sex at: | | sex at: | |
| | 10 min, p < 0.0001 | | 10 min, p < 0.0001 | |
| | 30 min, p < 0.0001 | | 30 min, p = 0.0004 | |
| | 1 hr, p = 0.0531 | | 1 hr, p = 0.0325 | |
| | 2 hr, p = 0.4818 | | 2 hr, p = 0.4716 | |
| | 4 hr, p = 0.9671 | | 4 hr, p = 0.9214 | |
| (2S,4R)- | F > M at 10 min | NS | F > M at 10 min | NS |
| | One-way ANOVA: | | One-way ANOVA: | |
| | effect of sex, F(1, 30) = 0.3430, | | effect of sex, F(1, 29) = 1.203, | |
| | p = 0.5625 | | p = 0.2811 | |
| | effect of time, F(4, 30) = 227.3, | | effect of time, F(4, 29) = 352.0, | |
| | p < 0.0001 | | p < 0.0001 | |
| | interaction, F(4, 30) = 2.883, | | interaction, F(4, 29) = 3.218, | |
| | p = 0.0393 | | p = 0.0265 | |
| | post-hoc comparison, effect of | | post-hoc comparison, effect of | |
| | sex at: | | sex at: | |
| | 10 min, p = 0.0152 | | 10 min, p = 0.0044 | |
| | 30 min, p = 0.8735 | | 30 min, p = 0.9715 | |
| | 1 hr, p = 0.7932 | | 1 hr, p = 0.9611 | |
| | 2 hr, p = 0.9813 | | 2 hr, p = 0.9829 | |
| | 4 hr, p = 0.9813 | | 4 hr, p > 0.9999 | |
| (5R)- | main effect of sex, M > F | M > F | NS | M > F |
| Me- | One-way ANOVA: | | One-way ANOVA: | |
| (2R,6R)- | effect of sex, F(1, 30) = 4.726, | | effect of sex, F(1, 30) = 0.3025, | |
| | p = 0.0377 | | p = 0.5864 | |
| | effect of time, F(4, 30) = 36.20, | | effect of time, F(4, 30) = 196.1, | |
| | p < 0.0001 | | p < 0.0001 | |
| | interaction, F(4, 30) = 1.424, | | interaction, F(4, 30) = 1.337, | |
| | p = 0.2502 | | p = 0.2790 | |

Note:
When a significant interaction (effect of sex × time) was detected in the one-way ANOVA, Holm-\check{S}ídák post-hoc comparison test was performed to compare the effect of sex at each time point. AUCs were compared using a standard t-test. Abbreviations: AUC, area under the concentration vs. time curve; F, female; HNK, hydroxynorketamine; M, male, NS, not statistically different. Abbreviations: AUC, area under the curve; F, female; HNK, hydroxynorketamine; M, male; NS, no statistical difference.

### Brain Concentrations of HNKs

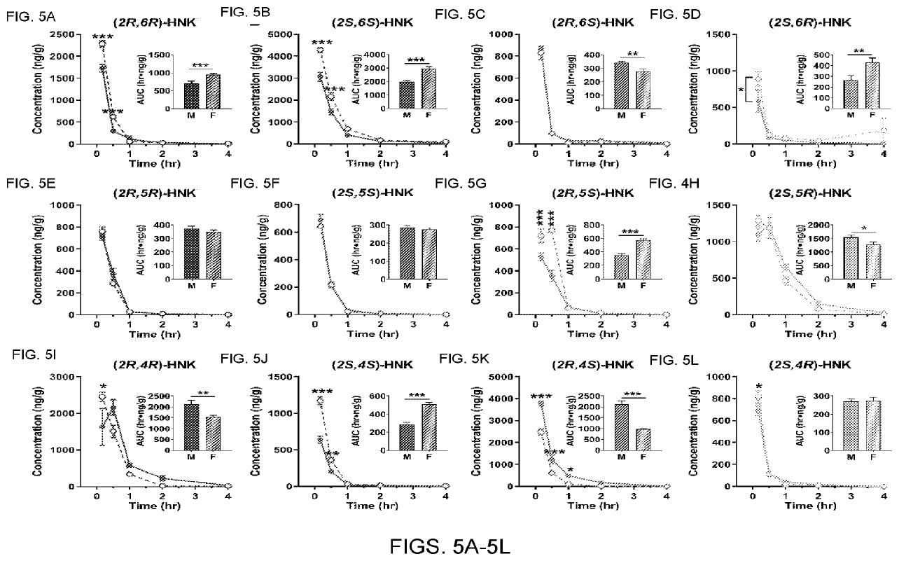

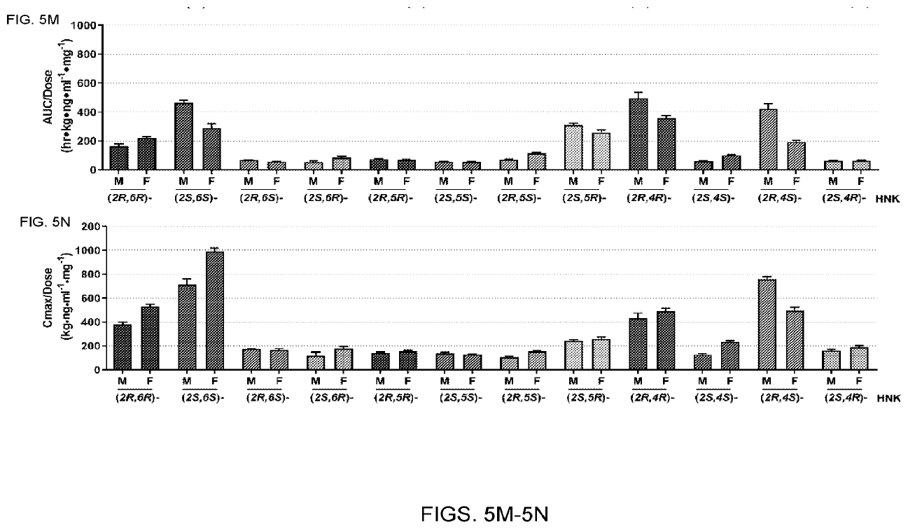

All 12 HNKs readily penetrated the brains of mice, where they were detected at 10 min following i.p. dosing (FIGS. 5A-5N). Peak brain levels were observed at the earliest sampling time point (10 min post-injection) in male and female mice for all HNKs, except for (2R,5S)-HNK in females, (2S,5R)-HNK in males, and (2R,4R)-HNK in males, where peak levels were observed at 30 min post-injection (FIGS. 5A-5N). Similar to the plasma, robust differences in effects of sex and compound administered were observed in the Cₘₐₓ and AUC were observed (see Table 8). Namely, approximately 7- and 8-fold differences were observed in the dose-normalized peak brain concentrations in male and female mice, respectively (Table 8). The highest brain levels (normalized to dose) were observed following (2S,6S)-HNK dosing in females and (2R,4S)-HNK in males, while the lowest were observed following (2R,4S)-HNK in males and (2S,5S)-HNK in females (Table 8). Approximately 9- and 12-fold differences were observed in the dose-normalized AUC of HNKs among male and female mice, respectively (Table 8). Males and females exhibited the highest AUC of (2R,4R)-HNK and (2S,6S)-HNK, respectively, and the lowest AUC of (2S,6R)-HNK and (2S,5S)-HNK, respectively. The AUC ratios of brain to plasma AUC of HNKs ranged between 0.6-1.3 for males and 0.7-1.4 for females (Table 8 and FIG. 1A-1D). Brain levels decreased rapidly, with elimination half-lives ranging between 0.3 and 0.8 h in both sexes (Table 8). Most HNKs could be detected in the brain up to 4 h post-treatment, with the following exceptions: (2S,5S)- and (2S,4R)-HNK were below LLOQs in both sexes, while (2R,6S)- and (2R,4S)-HNK were below LLOQs in females, but not males, at 4 h post-treatment.

Peak concentrations and AUCs of HNKs in the brain were also sex dependent (FIGS. 5A-5N and summarized in Table 9). Female mice had greater Cₘₐₓ and AUC of (2R,6R)-HNK (FIG. 5A), (2S,6S)-HNK (FIG. 5B), (2S,6R)-HNK (FIG. 5D), (2R,5S)-HNK (FIG. 5G), and (2S,4S)-HNK (FIG. 5J), compared to males. Although (2S,4R)-HNK resulted in higher brain concentrations in female mice during the first 10 min post-administration, no differences were observed in its AUC in between male and female mice (FIG. 5L).

Consistent with its plasma profile (FIG. 5I), (2R,4R)-HNK dosing resulted in higher brain levels in female than male mice during the first 10 min post-treatment, but AUC were higher in male mice (FIG. 5I). By contrast, male mice had greater Cₘₐₓ and AUC of (2R,4S)-HNK than females (FIG. 5K). Following systemic administration, AUC of (2R,6S)-HNK (FIG. 2C) and (2S,5R)-HNK (FIG. 5H) were greater in male mice relative to females, although no statistically significant sex-dependent differences in brain concentrations over time were observed. No sex-dependent differences were observed in Cₘₐₓ or AUC of (2R,5R)- and (2S,5S)-HNK (FIG. 5E and FIG. 5F, respectively).

The pharmacokinetic profile of (5R)-methyl-(2R,6R)-HNK was obtained revealing that, following intraperitoneal dosing, (5R)-methyl-(2R,6R)-HNK is detected in the plasma of mice between 10 min and 4 hr, with peak plasma levels observed at the earliest sampling time point, 10 min post-injection (FIG. 2D). The plasma elimination t_{1/2} was 0.8 hr in both sexes (Table 8). (5R)-methyl-(2R,6R)-HNK penetrated the brain, with peak brain levels observed at the earliest sampling timepoint, 10 min after administration (FIG. 2E). The brain to plasma AUC ratio was 0.4 and the brain elimination t_{1/2} was 0.8 hr in males and 0.7 hr in females (Table 8). Sex-dependent differences were observed in the plasma levels of (5R)-methyl-(2R,6R)-HNK, with an overall effect of sex detected in the plasma concentrations over time (FIG. 2D and Table 9), while no statistical difference was observed in brain concentrations over time (FIG. 2E and Table 9). The plasma and brain AUCs of (5R)-methyl-(2R,6R)-HNK were increased in male mice, relative to females (FIG. 2D and FIG. 2C and Table 9).

### Discussion

There are 12 unique HNKs that are formed in vivo following ketamine administration in mice and humans. This Example provides the first in-depth characterization of these 12 HNKs' pharmacokinetics following their direct administration to male and female mice. Overall, the data demonstrate that although HNKs exhibit robust differences in Cₘₐₓ and AUC in the plasma (Cₘₐₓ, ˜6-fold differences; AUC, ˜11-12-fold differences) and brains (Cₘₐₓ, 7-8-fold differences; AUC, 9-12-fold differences; Table 1) of mice, both rapid elimination profiles (half-lives ranging from 0.4-0.9 h in the plasma and 0.3-0.8 h in the brain; Table 8) and capacity to penetrate the brain (brain to plasma AUC ratios between 0.6-1.4, see Table 8) are conserved among the HNK stereoisomers. Notably, the ability of the 12 HNKs to rapidly penetrate the brain following systemic (i.p.) dosing suggests that these compounds would be capable of exerting pharmacodynamic effects in the brain, including antidepressant-relevant behavioral effects.

### Example 4: (2R,6R)-Hydroxynorketamine Use in the Treatment of Opioid Addiction

### General Methods

Animals: Male C₅₇BL/6J mice (8 weeks old, Jackson laboratories) were kept in a temperature and humidity-controlled environment and maintained in a 12:12 hr light/dark cycle (lights on at 7:00 AM). Mice were singly-housed for all the studies due to increased aggression following opioid administration and were randomly assigned to treatment groups.

Drugs: (2R,6R)-HNK hydrochloride was synthesized and characterized. Absolute and relative stereochemistry for (2R,6R)-HNK was confirmed by small molecule x-ray crystallography. For the intraperitoneal injections, drugs were dissolved in 0.9% saline and given in a volume of 5 ml/kg.

### Experiment 1: (2R,6R)-HNK Prevents the Development of Conditioning to Sub-Effective Doses of Morphine in Stress Susceptible Mice

### Methods

Social defeats: Socially defeated mice underwent a 10-day chronic social defeat stress paradigm. Briefly, experimental mice were introduced to the home cage (43 cm length×11 cm width×20 cm height) of a resident aggressive retired CD-1 breeder—prescreened for aggressive behaviors—for 10 min. Following this physical attack phase, mice were transferred and housed in the opposite side of the resident's cage divided by a perforated Plexiglas divider, in order to maintain continuous, 24 h, sensory contact. This process was repeated daily for 10 days, with experimental mice being introduced to a novel aggressive CD-1 mouse each day.

Social interaction test: Following the social defeats, on day 11, test mice were screened for susceptibility in a social interaction/avoidance choice test. The social interaction apparatus consisted of a rectangular three-chambered box (Stoelting Co., Wood Dale, IL, USA) comprised of two equal sized end-chambers and a smaller central chamber. The social interaction/avoidance choice test consisted of two 5-min phases. During the habituation phase, mice explored the empty apparatus. During the test phase, two small wire cages (Galaxy Cup, Spectrum Diversified Designs, Inc., Streetsboro, OH, USA), one containing a “stranger” CD-1 mouse and the other one empty, were placed in the far corners of each chamber. The time spent interacting (nose within close proximity of the cage) with the “stranger” mouse versus the empty cage was analyzed using TopScan video tracking software (CleverSys, Reston, Virginia). Mice with % Social Interaction ((Time spent interacting with stranger/total time interacting with the end-chamber cages)×100) greater than 50% were considered as resilient, whereas mice with <50% % Social Interaction were considered as susceptible.

Sucrose preference: Following the social interaction test, mice were assessed for sucrose preference in their home cages. Briefly, mice were singly housed and presented with two identical bottles containing either tap water or 1% (w/v) sucrose solution. Sucrose preference was measured (day 12) and were assigned to two groups: resilient (sucrose preference >70%) and susceptible (sucrose preference <55%).

Final determination of stress susceptibility: Only mice that showed susceptibility and resilience in both the social interaction and sucrose preference test continued with the rest of the experimental procedures. Out of 98 total mice tested, only 6 mice did not meet the same criteria for susceptibility in both the social interaction and sucrose preference tests. 44 mice were assigned as susceptible and 48 mice as resilient.

Test for spontaneous sucrose preference deficits reversal: In order to avoid having the effects of acute stress affecting the opioid conditioning outcomes, mice were allowed to recover from chronic social defeats stress for 20 days and re-tested for sucrose preference in their home cages (Day 30). Only mice that recovered normal sucrose preference (>70%) were used in the conditioning phase of the experiment. Only 3 mice that were initially assigned as resilient reverted sucrose preference to <70% and thus were excluded from the study. Therefore, 44 initially susceptible mice and 45 initially resilient mice were used in the morphine conditioning studies.

Sub-effective dose morphine conditioning: The conditioned-place preference (CPP) apparatus consisted of a rectangular three-chambered box (40 cm length×30 cm width×35 cm height; Stoelting Co., Wood Dale, IL, USA) comprised of two equal sized end-chambers (20 cm×18 cm×35 cm) and a central chamber (20 cm×10 cm×35 cm). One end-chamber had a perforated floor and plain black walls, whereas the other end-chamber had a smooth floor and walls with vertical black and white stripes. The CPP protocol consisted of a pre-conditioning phase, eight conditioning sessions, and a post-conditioning test. On Day 32 (pre-conditioning phase), stress susceptible and resilient mice (that had successfully recovered from their sucrose preference deficits) were placed in the CPP apparatus and were allowed to explore all compartments for a period of 20 min. Drug-paired compartment was assigned in a non-biased and random way. On Day 33, mice were given a single injection of saline or (2R,6R)-HNK (10 mg/kg), to assess for possible effects of HNK to prevent stress-susceptibility-induced enhancement of morphine CPP.

Conditioning phase (Days 34-37): During the morning sessions of the conditioning phase, saline was administered and mice were placed in their non-opioid assigned compartment for 40 min. Five hr later (afternoon sessions) saline, or morphine at the sub-effective dose of 0.25 mg/kg were administered, and mice were placed in their drug-paired compartment for 40 min. During the post-conditioning test session (i.e. Day 38), mice were placed in the CPP apparatus to freely explore all three compartments for 20 min. Time spent in each compartment was measured during both pre- and post-conditioning sessions using TopScan v2.0 (CleverSys, Inc, VA, USA).

### Results

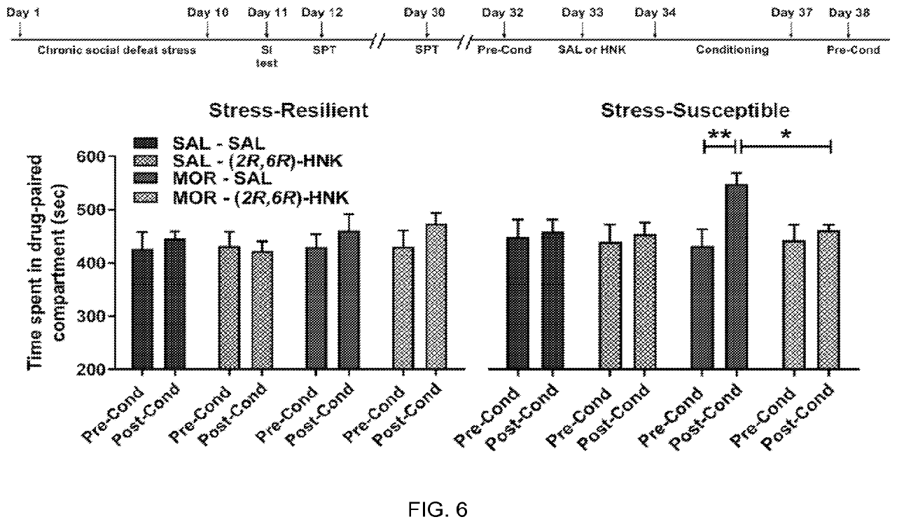

Stress susceptible mice (but not resilient mice) developed place preference to a sub-effective dose of morphine (i.e., 0.25 mg/kg), indicating that stress susceptibility to developing anhedonia and sociability deficits enhances the probability of developing conditioning to opioids at very low doses. A single administration of (2R,6R)-HNK prior to the conditioning phase of the CPP prevented morphine-induced place preference in stress susceptible mice, with no significant effect in stress-resilient mice (FIG. 6). (2R,6R)-HNK prevented conditioning to low doses of morphine in stress susceptible mice.

Experiment 2: (2R,6R)-HNK does not Prevent the Development of Conditioning to High Doses of Morphine

In order to determine whether the ability of (2R,6R)-HNK to prevent conditioning to sub-effective doses of morphine in stress susceptible mice was a result of its actions to reverse morphine conditioning per se, or a general reversal of the stress susceptible phenotype of mice, HNK's effects were assessed in stress-naïve mice.

### Methods

Morphine conditioned-place preference: The conditioned-place preference (CPP) apparatus consisted of a rectangular three-chambered box (40 cm length×30 cm width×35 cm height; Stoelting Co., Wood Dale, IL, USA) comprised of two equal sized end-chambers (20 cm×18 cm×35 cm) and a central chamber (20 cm×10 cm×35 cm). One end-chamber had a perforated floor and plain black walls, whereas the other end-chamber had a smooth floor and walls with vertical black and white stripes. The CPP protocol consisted of a pre-conditioning phase, eight conditioning sessions and a post-conditioning test. On Day 1 (pre-conditioning phase), mice were placed in the CPP apparatus and were allowed to explore all compartments for a period of 20 min. Drug-paired compartment was assigned in a non-biased and random way. On Day 2, mice were given a single injection of saline or (2R,6R)-HNK (10 mg/kg), to assess for possible effects of HNK to prevent morphine CPP.

Conditioning phase (Days 3-6): During the morning sessions of the conditioning phase, saline was administered and mice were placed in their non-opioid assigned compartment for 40 min. Five hr later (afternoon sessions) saline, or morphine (5 mg/kg) were administered, and mice were placed in their drug-paired compartment for 40 min. During the post-conditioning test session (i.e. Day 7), mice were placed in the CPP apparatus to freely explore all three compartments for 20 min. Time spent in each compartment was measured during both pre- and post-conditioning sessions using TopScan v2.0 (CleverSys, Inc, VA, USA).

### Results

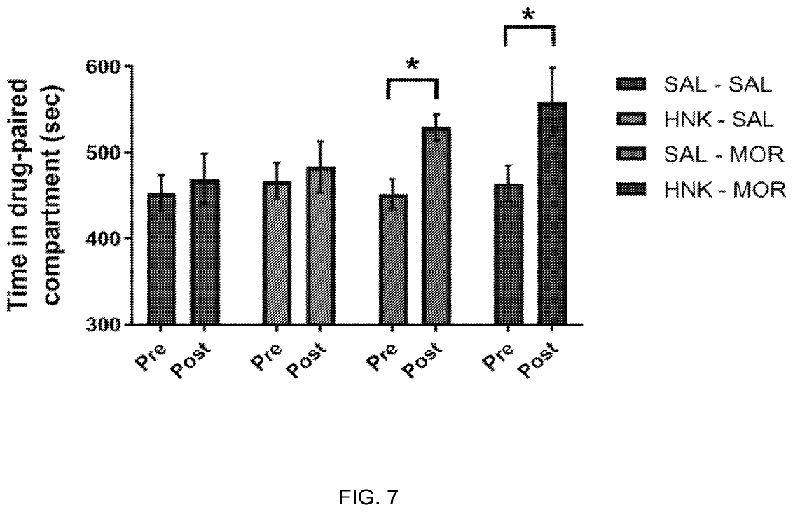

Administration of (2R,6R)-HNK did not prevent the development of high-dose (5 mg/kg) morphine CPP in mice, indicating that, in Experiment 1, HNK possibly reversed the stress-susceptibility phenotype of mice to reverse conditioning to sub-effective doses of morphine (FIG. 7).

### Experiment 3: (2R,6R)-HNK Prevents Conditioned-Place Aversion Induced by Precipitated Morphine Withdrawal in Opioid-Dependent Mice

### Methods

Precipitated morphine place aversion: The conditioned-place aversion (CPA) protocol was performed in a rectangular three-chambered box (40 cm length×30 cm width×35 cm height; Stoelting Co., Wood Dale, IL, USA) comprised of two equal sized end-chambers (20 cm×18 cm×35 cm) and a central chamber (20 cm×10 cm×35 cm). One end-chamber had a perforated floor and plain black walls, whereas the other end-chamber had a smooth floor and walls with vertical black and white stripes. The CPA protocol consisted of a pre-conditioning phase, four saline/morphine administration days in their home cages, a conditioning phase following morphine precipitated withdrawal in the CPA apparatus and a post-conditioning test. On Day 1 (pre-conditioning phase), mice were placed in the CPP apparatus and were allowed to explore all compartments for a period of 20 min. Naloxone-paired compartment was assigned to be in their most preferred compartment defined during the pre-conditioning test. On Days 2-5, mice were given a single injection of saline or morphine (10 mg/kg) in their home cages (same time as their pre-conditioning test). During the conditioning session (i.e. Day 5, 1 hr and 50 min after their last saline or morphine injection), mice were given a single injection of saline or (2R,6R)-HNK (10 mg/kg) and 10 min later were given another injection of either saline or the opioid receptor antagonist naloxone (1 mg/kg, i.p.) and immediately placed in the CPA apparatus and confined in their preferred compartment (based on the pre-conditioning phase) for 30 min. 24 hr later, mice were placed in the CPA apparatus and were allowed to freely explore all three compartments for 20 min. Time spent in each compartment was measured during both pre- and post-conditioning sessions using TopScan v2.0 (CleverSys, Inc, VA, USA).

### Results

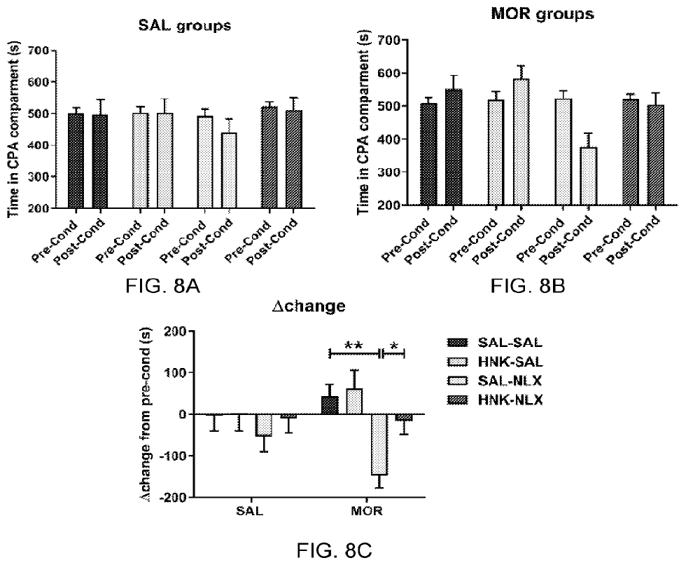

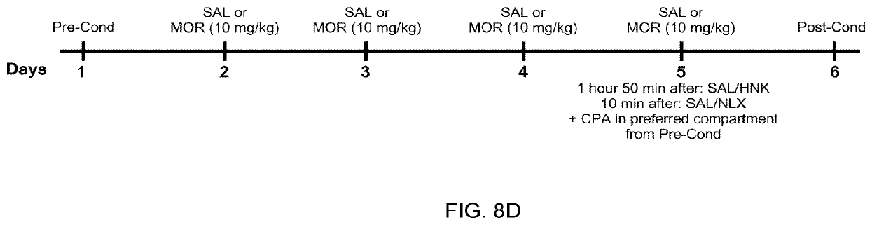

Administration of (2R,6R)-HNK completely prevented the development of naloxone-induced place aversion in morphine-dependent mice, and (2R,6R)-HNK prevents conditioned-place aversion (dysphoria) induced by acute morphine abstinence (FIGS. 8A-8D).

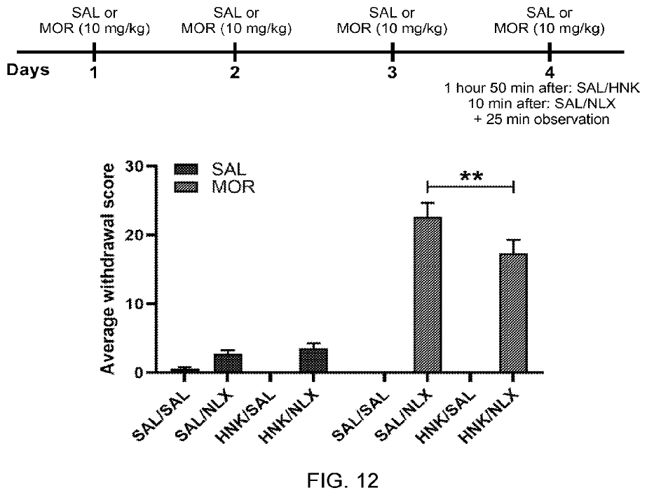

Administration of (2R,6R)-HNK decreases somatic symptoms following naloxone-precipitated morphine withdrawal (FIG. 12).

### Experiment 4: (2R,6R)-HNK Enhances Escape Behavior and Reduces Overall Precipitated Morphine Withdrawal Symptoms in Opioid-Dependent Mice

### Methods

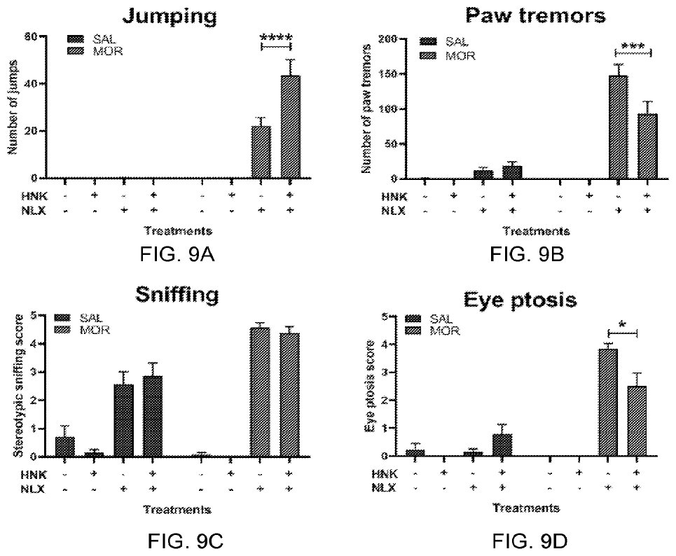

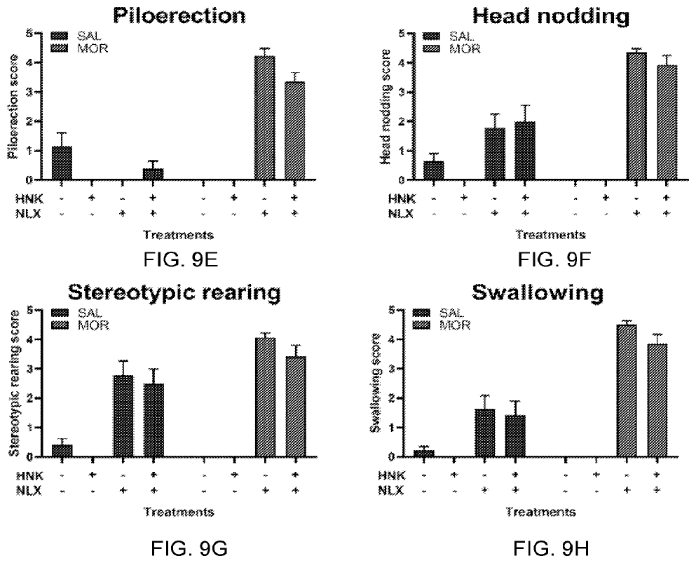

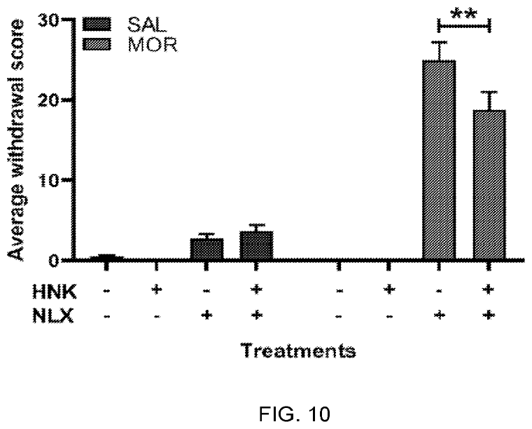

Precipitated morphine withdrawal symptoms: Mice were given a single injection of saline or morphine (10 mg/kg) in their home cages for 4 days. On Day 5, 1 hr and 50 min after their last saline or morphine injection, mice were given a single injection of saline or (2R,6R)-HNK (10 mg/kg) and 10 min later were given another injection of either saline or the opioid receptor antagonist naloxone (1 mg/kg, i.p.). The mice were immediately placed in plexiglass cylinders and their behaviors were monitored and live-scored for 25 min by an experimenter blind to any treatment conditions. Behaviors that were scored included: number of jumps (escape behavior), number of paw tremors, and occurrence (1 point) or not in each 5-min interval for the following symptoms: stereotypic sniffing, stereotypic rearing, eye ptosis, piloerection, head nodding and swallowing (FIGS. 9A-9H). An average score for each animal was given after analyzing all the withdrawal symptoms (FIG. 10).

### Results

Administration of (2R,6R)-HNK, while increasing the escape-related jumping behavior, decreased paw tremors and induced an overall reduction in average precipitated withdrawal symptoms of morphine (FIGS. 9A-9H and FIG. 10).

### Experiment 5: (2R,6R)-HNK Reverses Sub-Threshold Stress-Induced Behavioral Impairment in Mice Previously Exposed to Morphine

The aim of this experiment was to determine whether prior exposure to a rewarding dose of morphine (10 mg/kg) is able to induce a susceptibility phenotype in mice undergoing a sub-threshold social defeat stress. Then it was studied whether (2R,6R)-HNK prevents this behavioral impairment in mice that were exposed to opioids.

### Methods

Morphine administration: Mice were given a single injection of saline or morphine (10 mg/kg) in their home cages, for a total of 4 days. 10 days after their last saline or morphine injection, mice underwent a subthreshold social defeat stress protocol. 2 hours after stress, mice were given a single injection of saline or (2R,6R)-HNK (10 mg/kg).

Sub-threshold social defeats: On day 15, experimental mice were introduced to the home cage (43 cm length×11 cm width×20 cm height) of a resident aggressive retired CD-1 breeder—prescreened for aggressive behaviors—for 2 min. Following this physical attack phase, mice were transferred and housed in the opposite side of the resident's cage divided by a perforated Plexiglas divider, in order to maintain sensory contact for 15 min. This process was repeated within the same day 3 times, with experimental mice being introduced to a novel aggressive CD-1 mouse for each of those 3 trials. Control mice (non-stressed) were placed in a new social defeat cage that did not contain a CD-1 aggressive mouse in a separate room.

Social interaction test: Following the sub-threshold social defeat stress, on day 16, test mice were screened for susceptibility in a social interaction/avoidance choice test. The social interaction apparatus consisted of a rectangular three-chambered box (Stoelting Co., Wood Dale, IL, USA), comprised of two equal sized end-chambers and a smaller central chamber. The social interaction/avoidance choice test consisted of two 5-min phases. During the habituation phase, mice explored the empty apparatus. During the test phase, two small wire cages (Galaxy Cup, Spectrum Diversified Designs, Inc., Streetsboro, OH, USA), one containing a “stranger” CD-1 mouse and the other one empty, were placed in the far corners of each chamber. The time spent interacting (nose within close proximity of the cage) with the “stranger” mouse versus the empty cage was analyzed using TopScan video tracking software (CleverSys, Reston, Virginia). % Social Interaction was calculated as the time spent interacting with stranger divided by the total time interacting with the end-chamber cages×100.

Sucrose preference: Following the social interaction test, mice were assessed for sucrose preference in their home cages. Briefly, mice were singly housed and presented with two identical bottles containing either tap water or 1% (w/v) sucrose solution. Sucrose and water bottles were weighed on day 17 and % sucrose preference was calculated as the amount of sucrose consumed divided by the total liquid amount consumed (sucrose and water)×100.

Female urine sniffing test: After measuring sucrose (day 17), mice were placed (singly) in freshly made home cages for a habituation period of 10 min. Subsequently, one plain cotton tip was secured on the center of the cage wall and mice were allowed to sniff and habituate to the tip for a period of 30 min. Then, the plain cotton tip was removed and replaced by two cotton tip applicators, one infused with fresh female mouse estrus urine and the other with fresh male mouse urine. These applicators were presented and secured at the two corners of the cage wall simultaneously. Sniffing time for both female and male urine was scored by a trained observer for a period of three min. % Female urine preference was calculated as the time spent by the mouse sniffing the applicator containing the female urine divide by the total time the mouse spent sniffing both applicators ×100.

Mild stress reinstatement of maladaptive behaviors: Seven days after the female urine sniffing test (10 days after the sub-threshold social defeats), mice were re-screened for sucrose preference. All mice were then subjected to an 1-min exposure in the cage of an aggressive CD-1 mouse for assessing reinstatement of the previously acquired maladaptive behaviors (4 days after determination of sucrose preference restoration, i.e., Day 28). Following this exposure, mice were placed back in their home cages and presented with two identical bottles containing either tap water or 1% (w/v) sucrose solution for sucrose preference overnight. Next day, sucrose preference was determined again.

### Results

(2R,6R)-HNK reversed stress susceptibility phenotypes of mice previously exposed to morphine in the social interaction, sucrose preference, female urine sniffing, and untorn nestlet weight tests (FIGS. 11A-11D). Following 10 of sub-threshold social defeat stress, all mice recovered and had normal (>70%) sucrose preference and untorn nestlet weights (FIGS. 11E-11F). An acute 1 min re-exposure to an aggressive CD-1 mouse reinstated sucrose preference deficits in mice that were previously exposed to morphine, but HNK treatment after the first defeats was able to act prophylactically and prevent HNK-treated mice to show reinstatement of maladaptive behaviors after a brief re-exposure to stress (FIGS. 11G-11H).

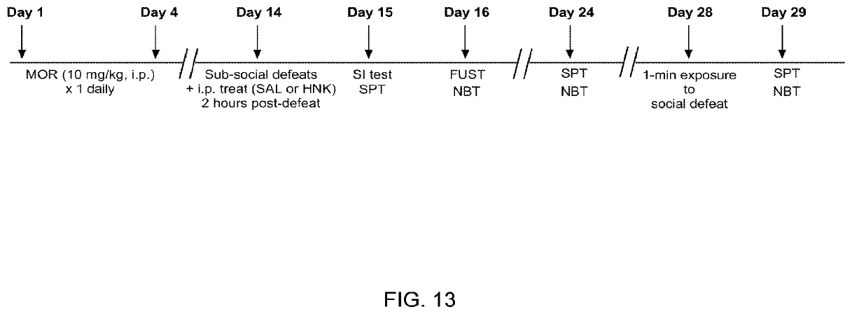

(2R,6R)-HNK reverses emotional deficits induced by stress during morphine abstinence and prevents reinstatement following recovery (FIGS. 11G-11H, FIG. 12, FIG. 13).

### REFERENCES

1. Abbott, J. A., & Popescu, G. K. (2020). Hydroxynorketamine Blocks NMDA Receptor Currents by Binding to Closed Receptors. Mol Pharmacol.
2. Aguilar-Valles A, De Gregorio D, Matta-Camacho E, Eslamizade M J, Khlaifia A, Skaleka A, Lopez-Canul M, Torres-Berrio A, Bermudez S, Rurak G M, Simard S, Salmaso N, Gobbi G, Lacaille J C and Sonenberg N (2021) Antidepressant actions of ketamine engage cell-specific translation via eIF4E. Nature 590:315-319.
3. Berman R M, Cappiello A, Anand A, Oren D A, Heninger G R, Charney D S and Krystal J H (2000) Antidepressant effects of ketamine in depressed patients. Biol Psychiatry 47:351-354.
4. Casarotto P C, Girych M, Fred S M, Kovaleva V, Moliner R, Enkavi G, Biojone C, Cannarozzo C, Sahu M P, Kaurinkoski K, Brunello C A, Steinzeig A, Winkel F, Patil S, Vestring S, Serchov T, Diniz C, Laukkanen L, Cardon I, Antila H, Rog T, Piepponen T P, Bramham C R, Normann C, Lauri S E, Saarma M, Vattulainen I and Castren E (2021) Antidepressant drugs act by directly binding to TRKB neurotrophin receptors. Cell.
5. Chen, B. K., Luna, V. M., LaGamma, C. T., Xu, X., Deng, S. X., Suckow, R. F., Cooper, T. B., Shah, A., Brachman, R. A., Mendez-David, I., David, D. J., Gardier, A. M., Landry, D. W., & Denny, C. A. (2020). Sex-specific neurobiological actions of prophylactic (R,S)-ketamine, (2R,6R)-hydroxynorketamine, and (2S,6S)-hydroxynorketamine. Neuropsychopharmacology, 45(9), 1545-1556.
6. Chou, D. (2020). Brain-derived neurotrophic factor in the ventrolateral periaqueductal gray contributes to (2R,6R)-hydroxynorketamine-mediated actions. Neuropharmacology, 170, 108068.
7. Chou, D., Peng, H. Y., Lin, T. B., Lai, C. Y., Hsieh, M. C., Wen, Y. C., Lee, A. S., Wang, H. H., Yang, P. S., Chen, G. D., & Ho, Y. C. (2018). (2R,6R)-hydroxynorketamine rescues chronic stress-induced depression-like behavior through its actions in the midbrain periaqueductal gray. Neuropharmacology, 139, 1-12.
8. Elmer, G. I., Tapocik, J. D., Mayo, C. L., Zanos, P., & Gould, T. D. (2020). Ketamine metabolite (2R,6R)-hydroxynorketamine reverses behavioral despair produced by adolescent trauma. Pharmacol Biochem Behav, 172973.
9. Farmer C A, Gilbert J R, Moaddel R, George J, Adeojo L, Lovett J, Nugent A C, Kadriu B, Yuan P, Gould T D, Park L T and Zarate C A, Jr. (2020) Ketamine metabolites, clinical response, and gamma power in a randomized, placebo-controlled, crossover trial for treatment-resistant major depression. Neuropsychopharmacology 45:1398-1404.
10. Fava M, Freeman M P, Flynn M, Judge H, Hoeppner B B, Cusin C, Ionescu D F, Mathew S J, Chang L C, Iosifescu D V, Murrough J, Debattista C, Schatzberg A F, Trivedi M H, Jha M K, Sanacora G, Wilkinson S T and Papakostas G I (2020) Correction: Double-blind, placebo-controlled, dose-ranging trial of intravenous ketamine as adjunctive therapy in treatment-resistant depression (TRD). Mol Psychiatry 25:1604.
11. Fukumoto, K., Fogaca, M. V., Liu, R. J., Duman, C., Kato, T., Li, X. Y., & Duman, R. S. (2019). Activity-dependent brain-derived neurotrophic factor signaling is required for the antidepressant actions of (2R,6R)-hydroxynorketamine. Proc Natl Acad Sci USA, 116(1), 297-302.
12. Highland, J. N., Morris, P. J., Zanos, P., Lovett, J., Ghosh, S., Wang, A. Q., Zarate, C. A., Jr., Thomas, C. J., Moaddel, R., & Gould, T. D. (2018). Mouse, rat, and dog bioavailability and mouse oral antidepressant efficacy of (2R,6R)-hydroxynorketamine. J Psychopharmacol, 269881118812095.
13. Highland I N, Zanos P, Riggs L M, Georgiou P, Clark S M, Morris P J, Moaddel R, Thomas C J, Zarate C A, Jr., Pereira E F R and Gould T D (in press) Hydroxynorketamines:
   pharmacology and potential therapeutic applications. Pharmacological Reviews.
14. Ho, M. F., Correia, C., Ingle, J. N., Kaddurah-Daouk, R., Wang, L., Kaufmann, S. H., & Weinshilboum, R. M. (2018). Ketamine and ketamine metabolites as novel estrogen receptor ligands: Induction of cytochrome P450 and AMPA glutamate receptor gene expression. Biochem Pharmacol, 152, 279-292.
15. Lilius T O, Viisanen H, Jokinen V, Niemi M, Kalso E A and Rauhala P V (2018) Interactions of (2S,6S;2R,6R)-Hydroxynorketamine, a Secondary Metabolite of (R,S)-Ketamine, with Morphine. Basic Clin Pharmacol Toxicol 122:481-488.
16. Leung, L. Y., & Baillie, T. A. (1986). Comparative pharmacology in the rat of ketamine and its two principal metabolites, norketamine and (Z)-6-hydroxynorketamine. J Med Chem, 29(11), 2396-2399.
17. Lumsden, E. W., Troppoli, T. A., Myers, S. J., Zanos, P., Aracava, Y., Kehr, J., Lovett, J., Kim, S., Wang, F. H., Schmidt, S., Jenne, C. E., Yuan, P., Morris, P. J., Thomas, C. J., Zarate, C. A., Jr., Moaddel, R., Traynelis, S. F., Pereira, E. F. R., Thompson, S. M., Albuquerque, E. X., & Gould, T. D. (2019). Antidepressant-relevant concentrations of the ketamine metabolite (2R,6R)-hydroxynorketamine do not block NMDA receptor function. Proc Natl Acad Sci USA, 116(11), 5160-5169.
18. Moaddel, R., Abdrakhmanova, G., Kozak, J., Jozwiak, K., Toll, L., Jimenez, L., Rosenberg, A., Tran, T., Xiao, Y., Zarate, C. A., & Wainer, I. W. (2013). Sub-anesthetic concentrations of (R,S)-ketamine metabolites inhibit acetylcholine-evoked currents in alpha7 nicotinic acetylcholine receptors. Eur J Pharmacol, 698(1-3), 228-234. S0014-2999(12)00954-5 [pii]
19. Moaddel R, Sanghvi M, Dossou K S, Ramamoorthy A, Green C, Bupp J, Swezey R, O'Loughlin K and Wainer I W (2015) The distribution and clearance of (2S,6S)-hydroxynorketamine, an active ketamine metabolite, in Wistar rats. Pharmacol Res Perspect 3:e00157.
20. Moaddel, R., Sanghvi, M., Ramamoorthy, A., Jozwiak, K., Singh, N., Green, C., O'Loughlin, K., Torjman, M., & Wainer, I. W. (2016). Subchronic administration of (R,S)-ketamine induces ketamine ring hydroxylation in Wistar rats. J Pharm Biomed Anal, 127, 3-8. https://doi.org/10.1016/j.jpba.2016.03.030
21. Moaddel, R., Venkata, S. L., Tanga, M. J., Bupp, J. E., Green, C. E., Iyer, L., Furimsky, A., Goldberg, M. E., Torjman, M. C., & Wainer, I. W. (2010). A parallel chiral-achiral liquid chromatographic method for the determination of the stereoisomers of ketamine and ketamine metabolites in the plasma and urine of patients with complex regional pain syndrome. Talanta, 82(5), 1892-1904.
22. Morris, P. J., Moaddel, R., Zanos, P., Moore, C. E., Gould, T. D., Zarate, C. A., Jr., & Thomas, C. J. (2017). Synthesis and N-Methyl-d-aspartate (NMDA) Receptor Activity of Ketamine Metabolites. Org Lett, 19(17), 4572-4575. https://doi.org/10.1021/acs.orglett.7b02177
23. Murrough J W, Perez A M, Pillemer S, Stern J, Parides M K, aan het Rot M, Collins K A, Mathew S J, Charney D S and Iosifescu D V (2013) Rapid and longer-term antidepressant effects of repeated ketamine infusions in treatment-resistant major depression. Biol Psychiatry 74:250-256.
24. Nelson, R. J., & Trainor, B. C. (2007). Neural mechanisms of aggression. Nat Rev Neurosci, 8(7), 536-546
25. Paul, R. K., Singh, N. S., Khadeer, M., Moaddel, R., Sanghvi, M., Green, C. E., O'Loughlin, K., Torjman, M. C., Bernier, M., & Wainer, I. W. (2014). (R,S)-Ketamine metabolites (R,S)-norketamine and (2S,6S)-hydroxynorketamine increase the mammalian target of rapamycin function. Anesthesiology, 121(1), 149-159.
26. Pham, T. H., Defaix, C., Xu, X., Deng, S. X., Fabresse, N., Alvarez, J. C., Landry, D. W., Brachman, R. A., Denny, C. A., & Gardier, A. M. (2018). Common Neurotransmission Recruited in (R,S)-Ketamine and (2R,6R)-Hydroxynorketamine-Induced Sustained Antidepressant-like Effects. Biol Psychiatry, 84(1), e3-e6.
27. Rahman, S. U., Hao, Q., He, K., Li, Y., Yang, X., Ye, T., Ali, T., Zhou, Q., & Li, S. (2020). Proteomic Study Reveals the Involvement of Energy Metabolism in the Fast Antidepressant Effect of (2R,6R)-Hydroxy Norketamine. Proteomics Clin Appl, e1900094.
28. Riggs, L. M., Aracava, Y., Zanos, P., Fischell, J., Albuquerque, E. X., Pereira, E. F. R., Thompson, S. M., & Gould, T. D. (2019). (2R,6R)-hydroxynorketamine rapidly potentiates hippocampal glutamatergic transmission through a synapse-specific presynaptic mechanism. Neuropsychopharmacology.
29. Sandbaumhuter, F. A., Theurillat, R., & Thormann, W. (2017). Separation of hydroxynorketamine stereoisomers using capillary electrophoresis with sulfated beta-cyclodextrin and highly sulfated gamma-cyclodextrin. Electrophoresis, 38(15), 1878-1885.
30. Shaffer, C. L., Dutra, J. K., Tseng, W. C., Weber, M. L., Bogart, L. J., Hales, K., Pang, J., Volfson, D., Am Ende, C. W., Green, M. E., & Buhl, D. L. (2019). Pharmacological evaluation of clinically relevant concentrations of (2R,6R)-hydroxynorketamine. Neuropharmacology, 153, 73-81.
31. Shen M and Machado S G (2017) Bioequivalence evaluation of sparse sampling pharmacokinetics data using bootstrap resampling method. J Biopharm Stat 27:257-264.
32. Yamaguchi, J. I., Toki, H., Qu, Y., Yang, C., Koike, H., Hashimoto, K., Mizuno-Yasuhira, A., & Chaki, S. (2018). (2R,6R)-Hydroxynorketamine is not essential for the antidepressant actions of (R)-ketamine in mice. Neuropsychopharmacology, 43(9), 1900-1907.
33. Ye, L., Ko, C. Y., Huang, Y., Zheng, C., Zheng, Y., & Chou, D. (2019). Ketamine metabolite (2R,6R)-hydroxynorketamine enhances aggression via periaqueductal gray glutamatergic transmission. Neuropharmacology, 157, 107667.
34. Yokoyama, R., Higuchi, M., Tanabe, W., Tsukada, S., Naito, M., Yamaguchi, T., Chen, L., Kasai, A., Seiriki, K., Nakazawa, T., Nakagawa, S., Hashimoto, K., Hashimoto, H., & Ago, Y. (2020). (S)-norketamine and (2S,6S)-hydroxynorketamine exert potent antidepressant-like effects in a chronic corticosterone-induced mouse model of depression. Pharmacol Biochem Behav, 191, 172876.
35. Zanos, P., Highland, J. N., Liu, X., Troppoli, T. A., Georgiou, P., Lovett, J., Morris, P. J., Stewart, B. W., Thomas, C. J., Thompson, S. M., Moaddel, R., & Gould, T. D. (2019). (R)-Ketamine exerts antidepressant actions partly via conversion to (2R,6R)-hydroxynorketamine, while causing adverse effects at sub-anaesthetic doses. Br J Pharmacol, 176(14), 2573-2592.
36. Zanos, P., Highland, J. N., Stewart, B. W., Georgiou, P., Jenne, C. E., Lovett, J., Morris, P. J., Thomas, C. J., Moaddel, R., Zarate, C. A., Jr., & Gould, T. D. (2019). (2R,6R)-hydroxynorketamine exerts mGlu2 receptor-dependent antidepressant actions. Proc Natl Acad Sci USA, 116(13), 6441-6450. ht
37. Zanos, P., Moaddel, R., Morris, P. J., Georgiou, P., Fischell, J., Elmer, G. I., Alkondon, M., Yuan, P., Pribut, H. J., Singh, N. S., Dossou, K. S., Fang, Y., Huang, X. P., Mayo, C. L., Wainer, I. W., Albuquerque, E. X., Thompson, S. M., Thomas, C. J., Zarate, C. A., Jr., & Gould, T. D. (2016). NMDAR inhibition-independent antidepressant actions of ketamine metabolites. Nature, 533(7604), 481-486.
38. Zarate, C. A., Jr., Brutsche, N., Laje, G., Luckenbaugh, D. A., Venkata, S. L., Ramamoorthy, A., Moaddel, R., & Wainer, I. W. (2012). Relationship of ketamine's plasma metabolites with response, diagnosis, and side effects in major depression. Biol Psychiatry, 72(4), 331-338.
39. Zarate C A, Jr., Singh J B, Carlson P J, Brutsche N E, Ameli R, Luckenbaugh D A, Charney D S and Manji H K (2006) A randomized trial of an N-methyl-D-aspartate antagonist in treatment-resistant major depression. Arch Gen Psychiatry 63:856-864.
40. Zhang, K., Toki, H., Fujita, Y., Ma, M., Chang, L., Qu, Y., Harada, S., Nemoto, T., Mizuno-Yasuhira, A., Yamaguchi, J. I., Chaki, S., & Hashimoto, K. (2018). Lack of deuterium isotope effects in the antidepressant effects of (R)-ketamine in a chronic social defeat stress model. Psychopharmacology (Berl), 235(11), 3177-3185.
41. Zhao, X., Venkata, S. L., Moaddel, R., Luckenbaugh, D. A., Brutsche, N. E., Ibrahim, L., Zarate, C. A., Jr., Mager, D. E., & Wainer, I. W. (2012). Simultaneous population pharmacokinetic modelling of ketamine and three major metabolites in patients with treatment-resistant bipolar depression. Br J Clin Pharmacol, 74(2), 304-314.

A number of patent and non-patent publications are cited herein in order to describe the state of the art to which this disclosure pertains. The entire disclosure of each of these publications is incorporated by reference herein.

While certain embodiments of the present disclosure have been described and/or exemplified above, various other embodiments will be apparent to those skilled in the art from the foregoing disclosure. The present disclosure is, therefore, not limited to the particular embodiments described and/or exemplified, but is capable of considerable variation and modification without departure from the scope and spirit of the appended claims.

1. A compound of any one of formula (I), formula (II), or formula (III), or a pharmaceutically acceptable salt, solvate, hydrate, cocrystal, or prodrug thereof: 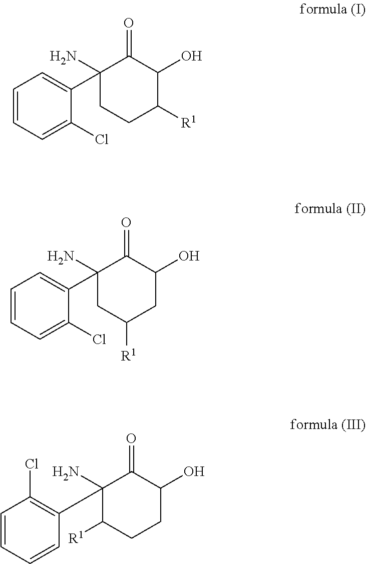
wherein in formula (I), formula (II), and formula (III):
R¹ is independently selected at each occurrence from halogen, hydroxyl, amino, cyano, C₁-C₄alkyl, C₁-C₄alkoxy, mono- and di-C₁-C₄alkylamino, C₁-C₂haloalkyl, and C₁-C₂haloalkoxy.

2. The compound of claim 1, wherein the compound of formula (I) is a compound of any one of formula (Ia), formula (Ib), formula (Ic), or formula (Id), or a pharmaceutically acceptable salt, solvate, hydrate, cocrystal, or prodrug thereof:

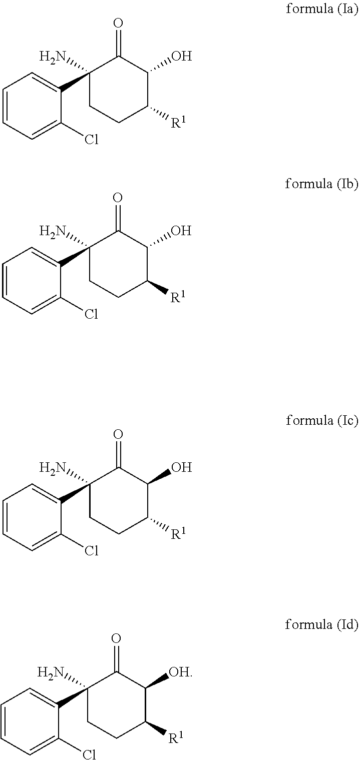

3. The compound of claim 1, wherein the compound of formula (II) is a compound of any one of formula (IIa), formula (IIb), formula (IIc), or formula (IId), or a pharmaceutically acceptable salt, solvate, hydrate, cocrystal, or prodrug thereof:

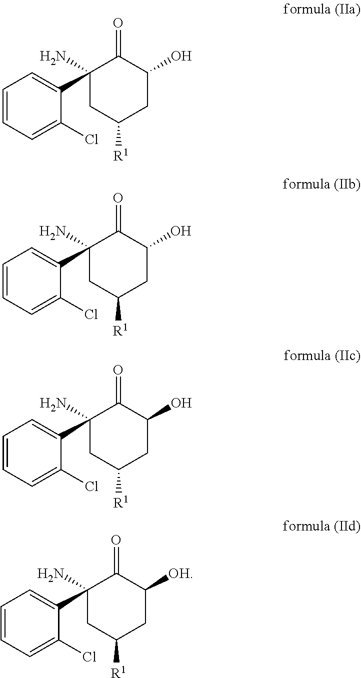

4. The compound of claim 1, wherein the compound of formula (III) is a compound of any one of formula (IIIa), formula (IIIb), formula (IIIc), or formula (IIId), or a pharmaceutically acceptable salt, solvate, hydrate, cocrystal, or prodrug thereof:

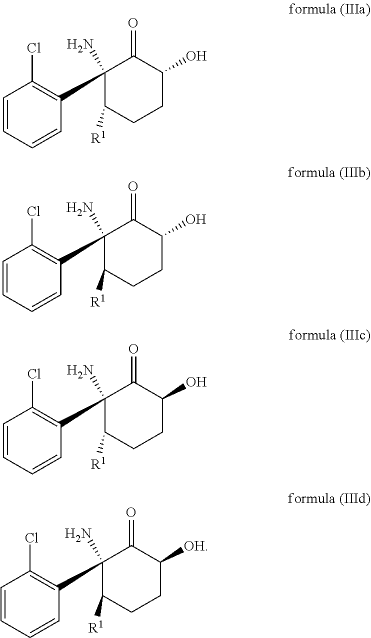

5. The compound of claim 1, wherein R¹ is independently selected at each occurrence from C₁-C₄alkyl.

6. The compound of claim 5, wherein R¹ is methyl.

7. The compound of claim 1, wherein the compound is of any one of formulas 1001-1084, or a pharmaceutically acceptable salt, solvate, hydrate, cocrystal, or prodrug thereof:

| 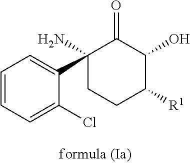 | |
|---|---|
| Compound # | R¹ |
| 1001 | —CH₃ |
| 1002 | —CH₂CH₃ |
| 1003 | —CH₂CH₂CH₃ |
| 1004 | —(CH)(CH₃)₂ |
| 1005 | —CH₂CH₂CH₂CH₃ |
| 1006 | —CH₂(CH)(CH₃)₂ |
| 1007 | —(CH₃)₃ |
| 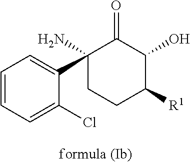 | |
| Compound # | R¹ |
| 1008 | —CH₃ |
| 1009 | —CH₂CH₃ |
| 1010 | —CH₂CH₂CH₃ |
| 1011 | —(CH)(CH₃)₂ |
| 1012 | —CH₂CH₂CH₂CH₃ |
| 1013 | —CH₂(CH)(CH₃)₂ |
| 1014 | —(CH₃)₃ |
| 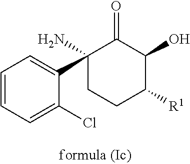 | |
| Compound # | R¹ |
| 1015 | —CH₃ |
| 1016 | —CH₂CH₃ |
| 1017 | —CH₂CH₂CH₃ |
| 1018 | —(CH)(CH₃)₂ |
| 1019 | —CH₂CH₂CH₂CH₃ |
| 1020 | —CH₂(CH)(CH₃)₂ |
| 1021 | —(CH₃)₃ |
| 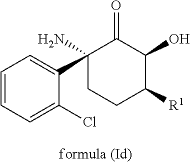 | |
| Compound # | R¹ |
| 1022 | —CH₃ |
| 1023 | —CH₂CH₃ |
| 1024 | —CH₂CH₂CH₃ |
| 1025 | —(CH)(CH₃)₂ |
| 1026 | —CH₂CH₂CH₂CH₃ |
| 1027 | —CH₂(CH)(CH₃)₂ |
| 1028 | —(CH₃)₃ |
| 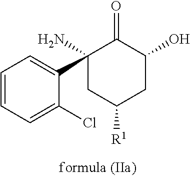 | |
| Compound # | R¹ |
| 1029 | —CH₃ |
| 1030 | —CH₂CH₃ |
| 1031 | —CH₂CH₂CH₃ |
| 1032 | —(CH)(CH₃)₂ |
| 1033 | —CH₂CH₂CH₂CH₃ |
| 1034 | —CH₂(CH)(CH₃)₂ |
| 1035 | —(CH₃)₃ |
| 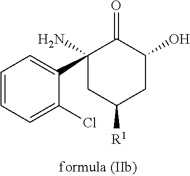 | |
| Compound # | R¹ |
| 1036 | —CH₃ |
| 1037 | —CH₂CH₃ |
| 1038 | —CH₂CH₂CH₃ |
| 1039 | —(CH)(CH₃)₂ |
| 1040 | —CH₂CH₂CH₂CH₃ |
| 1041 | —CH₂(CH)(CH₃)₂ |
| 1042 | —(CH₃)₃ |
| 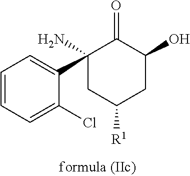 | |
| Compound # | R¹ |
| 1043 | —CH₃ |
| 1044 | —CH₂CH₃ |
| 1045 | —CH₂CH₂CH₃ |
| 1046 | —(CH)(CH₃)₂ |
| 1047 | —CH₂CH₂CH₂CH₃ |
| 1048 | —CH₂(CH)(CH₃)₂ |
| 1049 | —(CH₃)₃ |
| 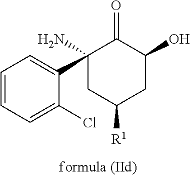 | |
| Compound # | R¹ |
| 1050 | —CH₃ |
| 1051 | —CH₂CH₃ |
| 1052 | —CH₂CH₂CH₃ |
| 1053 | —(CH)(CH₃)₂ |
| 1054 | —CH₂CH₂CH₂CH₃ |
| 1055 | —CH₂(CH)(CH₃)₂ |
| 1056 | —(CH₃)₃ |
| 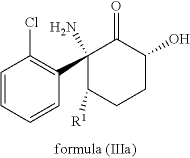 | |
| Compound # | R¹ |
| 1057 | —CH₃ |
| 1058 | —CH₂CH₃ |
| 1059 | —CH₂CH₂CH₃ |
| 1060 | —(CH)(CH₃)₂ |
| 1061 | —CH₂CH₂CH₂CH₃ |
| 1062 | —CH₂(CH)(CH₃)₂ |
| 1063 | —(CH₃)₃ |
| 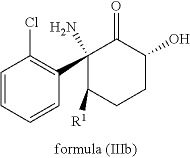 | |
| Compound # | R¹ |
| 1064 | —CH₃ |
| 1065 | —CH₂CH₃ |
| 1066 | —CH₂CH₂CH₃ |
| 1067 | —(CH)(CH₃)₂ |
| 1068 | —CH₂CH₂CH₂CH₃ |
| 1069 | —CH₂(CH)(CH₃)₂ |
| 1070 | —(CH₃)₃ |
| 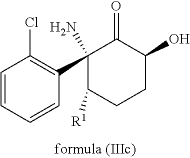 | |
| Compound # | R¹ |
| 1071 | —CH₃ |
| 1072 | —CH₂CH₃ |
| 1073 | —CH₂CH₂CH₃ |
| 1074 | —(CH)(CH₃)₂ |
| 1075 | —CH₂CH₂CH₂CH₃ |
| 1076 | —CH₂(CH)(CH₃)₂ |
| 1077 | —(CH₃)₃ |
| 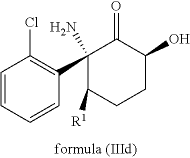 | |
| Compound # | R¹ |
| 1078 | —CH₃ |
| 1079 | —CH₂CH₃ |
| 1080 | —CH₂CH₂CH₃ |
| 1081 | —(CH)(CH₃)₂ |
| 1082 | —CH₂CH₂CH₂CH₃ |
| 1083 | —CH₂(CH)(CH₃)₂ |
| 1084 | —(CH₃)₃ |

8. The compound of claim 1, wherein the compound is compound 1001, or a pharmaceutically acceptable salt, solvate, hydrate, cocrystal, or prodrug thereof:

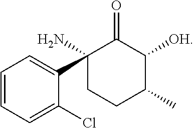 [SMILES: C[C@@H]1CC[C@@](N)(c2ccccc2Cl)C(=O)[C@@H]1O]

9. A pharmaceutical composition comprising one or more compounds according to claim 1, or a pharmaceutically acceptable salt, solvate, hydrate, cocrystal, or prodrug thereof, and a pharmaceutically acceptable carrier.

10.-13. (canceled)

14. The pharmaceutical composition of claim 9, wherein the pharmaceutical composition is formulated for oral administration.

15. A method of treating or preventing a disease or condition selected from a depressive disorder, an anxiety disorder, drug addiction, schizophrenia, Alzheimer's dementia, amyotrophic lateral sclerosis, complex regional pain syndrome (CRPS), chronic pain, neuropathic pain, anhedonia, and fatigue, the method comprising administering to the patient a therapeutically effective amount of a compound of claim 1 or a compound of any one of formulas 2001 to 2004, or a pharmaceutically acceptable salt, solvate, hydrate, cocrystal, or prodrug thereof:

| Compound # | R¹ |
|---|---|
| 2001 | 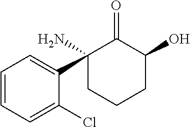 |
| 2002 | 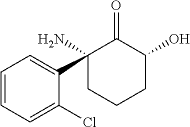 |
| 2003 | 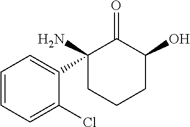 |
| 2004 | 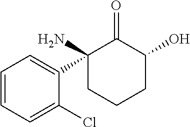 |

16. The method of claim 15, wherein the depressive disorder is selected from major depressive disorder, bipolar disorder, postpartum depression, drug withdrawal induced depression, treatment refractory depression, persistent depressive disorder (dysthymia), perinatal depression, seasonal affective disorder, seasonal depression, premenstrual dysphoric disorder, psychotic depression, suicidal ideation, suicidal actions, disruptive mood dysregulation disorder, premenstrual dysphoric disorder, substance/medication-induced depressive disorder, depressive disorder due to another medical condition, other specified depressive disorder, and an unspecified depressive disorder.

17. The method of claim 15, wherein the anxiety disorder is selected from general anxiety disorder, obsessive-compulsive disorder (OCD), post-traumatic stress disorder (PTSD), separation anxiety disorder, selective mutism, specific phobia, social anxiety disorder (social phobia), panic disorder, panic attack (specifier), agoraphobia, substance/medication-induced anxiety disorder, anxiety disorder due to another medical, other specified anxiety disorder, anhedonia, and unspecified anxiety disorder.

18. The method of claim 15, wherein the drug addiction is selected from nicotine addiction, alcohol addiction, cannabis addiction, cocaine addiction, and opioid addiction.

19. The method of claim 18, wherein the drug addiction is opioid addiction.

20. The method of claim 15, wherein the compound is a compound of formula 2001, or a pharmaceutically acceptable salt, solvate, hydrate, cocrystal, or prodrug thereof.

21. The method of claim 15, wherein the compound is a compound of formula 2002, or a pharmaceutically acceptable salt, solvate, hydrate, cocrystal, or prodrug thereof.
